(19) United States
(12) Patent Application Publication
(10) Pub. No.: US 2026/0242759 A1
(43) Pub. Date: Aug. 20, 2026

(54) GQ/11 Protein Peptidomimetics

(71) Applicant: Vrije Universiteit Brussel, Brussel (BE)

(72) Inventors: Steven Ballet, Putte (BE); Charlotte Martin, Brussel (BE); Morgane Mannes, Brussel (BE)

(21) Appl. No.: 18/876,800

(22) PCT Filed: Jun. 22, 2023
(86) PCT No.: PCT/EP2023/067016

§ 371 Date: Dec. 19, 2024

(30) Foreign Application Priority Data
Jun. 22, 2022 (EP) 22180492.5

(51) Int. Cl. C12N 9/14; C07K 1/30; G01N 33/74

(52) U.S. Cl. CPC C12N 9/14; C07K 1/306; G01N 33/74; G01N 2333/726; G01N 2333/914

(57) ABSTRACT

The present invention relates to G protein peptidomimetics, in particular G_{q/11} protein peptidomimetics, capable of stabilizing a GPCR, in particular a G_{q/11} protein-coupled receptor, in an active conformational state. The G protein peptidomimetics are derived from the α₅ helix of Gα_{q/11} protein or mini-G_{q} protein, in particular they arise from modifications of peptides comprising or consisting of the amino acid sequence set forth in SEQ ID NO:13 or SEQ ID NO:14. The invention further provides complexes of the G protein peptidomimetics and a GPCR, fusion polypeptides of a GPCR and the G protein peptidomimetics and compositions comprising the same. Further disclosed herein are uses of the G protein peptidomimetics, complexes, fusion polypeptides and compositions for determining the structure of a GPCR conformer, for screening for compounds capable of specifically binding to a GPCR conformer and as allosteric modulator of a GPCR and as a biosensor.

### CROSS REFERENCE TO RELATED APPLICATIONS

This application is a national phase entry under 35 U.S.C. § 371 of International Patent Application PCT/EP2023/067016, filed Jun. 22, 2023, designating the United States of America and published in English as International Patent Publication WO2023/247717 on Dec. 28, 2023, which claims the benefit under Article 8 of the Patent Cooperation Treaty to European Patent Application Serial No. 22180492.5, filed Jun. 22, 2022, the entireties of which are hereby incorporated by reference.

### INCORPORATION BY REFERENCE

The ST.26 XML Sequence listing named “10511-10697-US—Sequence Listing ST26”, created on Jun. 22, 2023, and having a size of 139,264 bytes, is hereby incorporated herein by this reference in its entirety

### TECHNICAL FIELD

The application generally relates to structural biology of G protein-coupled receptors (GPCRs). In particular, the present invention is directed to G_{q/11} protein peptidomimetics capable of stabilizing a GPCR, in particular a G_{q/11} protein-coupled receptor, in an active conformational state. Further disclosed herein are uses of the G_{q/11} protein peptidomimetics for determining the structure of a GPCR conformer, for screening for compounds capable of specifically binding to a GPCR conformer, as allosteric modulators of a GPCR and as biosensors.

### BACKGROUND

Over the past 20 years, enormous structural information on GPCRs has been obtained through X-ray crystallography and cryo-electron microscopy (cryo-EM), which has contributed to the understanding of the molecular mechanism of GPCRs. One of the hallmark achievements was the crystallization of the β₂ adrenergic receptor (β₂AR) in complex with the heterotrimeric Gₛ protein, being stabilized by a Confobody (Rasmussen et al. 2011. Nature 477:549-555). While several cryo-EM structures of GPCRs coupled to the (engineered) G_{i/o} protein were already reported, only a few GPCR-G_{q} (also referred to as G_{q/11} because of its closely related homologue G₁₁ that is 90% identical) complexes are available.

The first cryo-EM structures of GPCR-G_{q/11} complexes were solved thanks to the discovery of a single-chain variable fragment scFv16, originally developed to stabilize the rhodopsin-Gᵢ complex for crystallization (Maeda et al. 2018. Nat. Commun. 9:1-9). Therefore, G_{q/11} chimeras were generated in which the N-terminus of Gα_{q/11} was replaced by the N-terminus of Gαᵢ (αN helix). This strategy enabled the use of scFv16. The latter couples the αN to the β-subunit of the Gβγ-dimer, eventually stabilizing the nucleotide-free GPCR-G protein complex for cryo-EM. For some of the receptors an extra NanoBiT® system, a protein fragment complementation method, was necessary for stabilization. It consisted of the fusion of the receptor C-terminus to the large part of NanoBiT® (LgBiT) and the C-terminus of GP to the peptide of NanoBit® (HiBiT), altogether resulting in the generation of the functional NanoBiT® upon GPCR-G protein complexation (Xia et al. 2021. Nat. Commun. 12:1-9).

The first G_{q/11}-GPCR structure solved by cryo-EM was the muscarinic acetylcholine receptor 1 (M1R), which plays a role in the nervous system and is targeted in view of treating diseases such as Alzheimer's disease and schizophrenia. The structure of M1R in complex with G₁₁ was compared with a muscarinic receptor from the same subfamily, the muscarinic acetylcholine 2 receptor, coupled to Gₒ. From this analysis, some differences have been noted, such as the extension of transmembrane 5 (TM5), presenting an increased interaction with the G₁₁ protein (Maeda et al. 2019. Science 364:552-557). Later, the structure of the human histamine 1 receptor (H1R), involved in allergy and inflammation, was solved in complex with G_{q} by cryo-EM (Xia et al. 2021). Interestingly, this structure could potentially help in the development of more effective antihistamine drugs with fewer side effects. More recently, the structure of the cholecystokinin receptor (CCK_{B}R) in complex with G_{q} had also been determined (Zhang et al. 2021. Nat. Chem. Biol. 1-8). The latter receptor is of therapeutic value, given its crucial role in food intake and appetite regulation. Interestingly, the structure of a G_{q}-coupled 5-HT_{2A} serotonin receptor (5-HT_{2A}R) had been elucidated through cryo-EM (Kim et al. 2020. Cell 182:1574-1588). To obtain this cryo-EM structure, a complex was formed with a mini-Gα_{q}-βγ heterotrimer. Mini-G proteins have been very important tools to overcome the inherent instability and flexibility of these complexes. These mini-G proteins are often expressed together with the βγ-dimer to also investigate their interactions with the receptor. Contrary to the mini-Gₛ, the developed mini-G_{q} (based on the G_{q} protein) was unsuccessfully expressed in E. coli, probably due to an improper folding or instability reasons. Therefore, the strategy to obtain a stable mini-G_{q} variant consisted of transferring the amino acids crucial for G_{q} binding, especially at the C-terminus, onto the more stable mini-Gₛ. As such, several mini-G_{s/q} chimera were evaluated for binding to G_{q}-coupled receptors and loss of binding to GS-coupled receptors, resulting in the mini-G_{s/q}70, which contained 7 point mutations in the α₅ helix (R380K, Q384L, R385Q, H387N, Q390E, E392N and L394V) (Nehme et al. 2017. PLoS One 12:e0175642). This engineered mini-G_{q} protein strategy (based on mini-Gₛ) has been used to publish cryo-EM structures of several G_{q/11}-coupled receptors such as the ghrelin receptor (GHSR) (Wang et al. 2021. Molecular recognition of an acyl-peptide hormone and activation of ghrelin receptor. bioRxiv), the bradykinin receptors 1 and 2 (B1R and B2R) (Yin et al. 2021. Molecular basis for kinin selectivity and activation of the human bradykinin receptors. bioRxiv), orexin receptor 2 (OX2R) (Hong et al. 2021. Nat. Commun. 12:1-11), cholecystokinin 1 receptor (CCK1) (Mobbs et al. 2021. PLoS Biol. 19:e3001295), neurokinin-1 receptor (NK1R) (Harris et al. 2021. Selective G protein signaling driven by Substance P-Neurokinin Receptor structural dynamics. bioRxiv) and mass-related G protein-coupled receptors X2 and X4 (MRGPRX2 and MRGPRX4) (Cao et al. 2021. Nature: 1-6). To be able to form a stable complex with a GPCR, mini-G proteins need to be engineered, which may be a time-consuming process.

Because no X-ray crystal structures and only a few cryo-EM structures were obtained recently for G_{q/11}-coupled receptors in active conformation, it would be highly valuable to develop further tools capable to bind and stabilize G_{q/11} protein-coupled receptors amongst others for structural studies and drug discovery, which are preferably easy and cheap to generate and purify. Also preferable are small-sized tools, which are particularly advantageous for structural analyses such as nuclear magnetic resonance (NMR).

Confobodies were established to be crucial tools for structural biology and drug discovery. Unfortunately, their development and purification is a time-consuming and expensive process.

PCT/EP2021/086733 discloses the development and validation of Gₛ peptidomimetics, which have been shown to successfully bind and stabilize GS-coupled receptors.

### SUMMARY OF THE INVENTION

The present invention is based, at least in part, on the finding that peptides derived from the α₅ helix of Gα_{q/11} protein or mini-G_{q} protein, which comprise a staple and/or which comprise a C-terminal modification, preferably a substitution of a C-terminal residue, in particular the penultimate leucine residue, by an alanine analogue as defined herein below can bind a GPCR, in particular a G_{q/11} protein-coupled receptor, and increase agonist affinity to the receptor. This allows their use to stabilize the GPCR in an active conformational state to perform structure determination or fragment-based screening for drug discovery, or their use as allosteric modulators of the GPCR or as biosensors. Advantageously, the G protein peptidomimetics, in particular the G_{q/11} protein peptidomimetics, as described herein can be developed fast, their synthesis is cheap and allows easy modifications. Also advantageous is that the G protein peptidomimetics, in particular the G_{q/11} protein peptidomimetics, as described herein, were found to maintain their stabilizing properties in a cellular context.

As further shown in the experimental section, modification of the G protein peptidomimetics, in particular the G_{q/11} protein peptidomimetics, as described herein, by addition of a cell-penetrating peptide (CPP) increased cell permeability properties of the G protein peptidomimetics, while their stabilizing properties were maintained.

In particular, the invention relates to one or any combination of one or more of the below numbered aspects and embodiments with any other aspects, statement and/or embodiments:
1. A G protein peptidomimetic or salt thereof comprising or consisting of a sequence of the structure (I):

(I)
(SEQ ID NO: 21)
FX₂X₃X₄KDX₇ILQX₁₁NLX₁₄X₁₅X₁₆X₁₇X₁₈X₁₉

wherein X₂ is asparagine (N) or alanine (A);
wherein X₃ is selected from the group consisting of: aspartic acid (D), alanine (A), an amino acid containing an azidated side-chain, an amino acid residue containing an alkynyl side-chain, an amino acid containing a carboxylic acid group side-chain, an amino acid containing an amine side-chain, an amino acid containing a thiol side-chain and an olefinic amino acid;
wherein X₄ is cysteine (C) or valine (V), or an amino acid without a thiol side-chain;
wherein X₇ is selected from the group consisting of: isoleucine (I), threonine (T), an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid;
wherein X₁₁ is selected from the group consisting of: methionine (M), leucine (L), an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid;
wherein X₁₄ is selected from the group consisting of: arginine (R), lysine (K), an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid;
wherein X₁₅ is glutamic acid (E), homoglutamic acid or aspartic acid (D);
wherein X₁₆ is tyrosine (Y) or an aromatic amino acid selected from the group comprising or consisting of 4′-guanidinophenylalanine (Phe(4′guanidino)), tryptophan (W), phenylalanine (F), naphthylalanine, 1-naphthylalanine (1-NaI) and 2-naphthylalanine (2-NaI);
wherein X₁₇ is asparagine (N) or cysteine (C);
wherein X₁₈ is leucine (L), glutamic acid (E), or an alanine analogue, phenylalanine (F), tyrosine (Y), or tryptophan (W), wherein the alanine analogue is a molecule resulting from the replacement of at least one hydrogen of an alanine by at least one moiety selected from the group comprising C₆₋₁₂ cycloalkyl, C₆₋₁₂ aryl, heteroaryl, and C₆₋₁₂ cycloalkenyl; each moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl; and
wherein X₁₉ is selected from the group consisting of valine (V), a hydrophobic amino acid, or an acidic amino acid (such as aspartic acid (D), glutamic acid(E), D-aspartic acid or D-glutamic acid),
wherein the peptidomimetic comprises a covalent tether formed between X₃ and X₇, or between X₇ and X₁₁, or between X₁₁ and X₁₄, or between X₇ and X₁₄, from the reaction of an amino acid containing an azidated side-chain with an amino acid containing an alkynyl side-chain, or from the reaction of an amino acid containing an amine side-chain with an amino acid containing a carboxylic acid group side-chain, or from the reaction between two olefinic amino acids, or from the reaction between two amino acids each containing a thiol group side-chain, wherein said covalent tether is not part of the linear peptide backbone, and/or
wherein the sequence X₁₅X₁₆X₁₇X₁₈X₁₉ (SEQ ID NO:34) is not EYNLV (SEQ ID NO:35).
2. The G protein peptidomimetic or salt thereof according to 1, comprising a sequence of the structure (II) or (111):

(II)
(SEQ ID NO: 22)
FNX₃X₄KDX₇ILQX₁₁NLX₁₄X₁₅X₁₆X₁₇X₁₈X₁₉
(III)
(SEQ ID NO: 23)
FAX₃VKDX₇ILQX₁₁NLX₁₄X₁₅X₁₆X₁₇X₁₈X₁₉

wherein X₃, X₄, X₇, X₁₁, X₁₄, X₁₅, X₁₆, X₁₇, X₁₈ and X₁₉ are as defined in 1.
3. The G protein peptidomimetic or salt thereof according to 1 or 2, wherein the peptidomimetic comprises a covalent tether formed between X₃ and X₇ from the reaction of an amino acid containing an azidated side-chain with an amino acid containing an alkynyl side-chain, or from the reaction of an amino acid containing an amine side-chain with an amino acid containing a carboxylic acid group side-chain, or from the reaction between two olefinic amino acids, or from the reaction between two amino acids each containing a thiol group side-chain, preferably from the reaction of an amino acid containing an azidated side-chain with an amino acid containing an alkynyl side-chain, wherein said covalent tether is not part of the linear peptide backbone.
4. The G protein peptidomimetic or salt thereof according to any one of 1 to 3, wherein the peptidomimetic comprises a covalent tether formed from the reaction of the side-chain of X₃ with the side-chain of X₇, wherein X₇ is an amino acid containing an azidated side-chain and X₃ is an amino acid containing an alkynyl side-chain, wherein X₃ is an amino acid containing an azidated side-chain and X₇ is an amino acid containing an alkynyl side-chain, wherein X₃ is an amino acid containing a carboxylic acid group side-chain and X₇ is an amino acid containing an amine side-chain, wherein X₇ is an amino acid containing a carboxylic acid group side-chain and X₃ is an amino acid containing an amine side-chain, wherein X₃ and X₇ are olefinic amino acids, or wherein X₃ and X₇ are amino acids containing a thiol side-chain.
5. The G protein peptidomimetic or salt thereof according to any one of 1 to 4, comprising a sequence of the structure (IV) or (V):

(IV)
(SEQ ID NO: 24)
FNX₃X₄KDX₇ILQMNLRX₁₅X₁₆X₁₇X₁₈X₁₉
(V)
(SEQ ID NO: 25)
FAX₃VKDX₇ILQLNLKX₁₅X₁₆X₁₇X₁₈X₁₉,

wherein X₃ is selected from the group consisting of: an amino acid containing an azidated side-chain, an amino acid residue containing an alkynyl side-chain, an amino acid containing a carboxylic acid group side-chain, an amino acid containing an amine side-chain, an amino acid containing a thiol side-chain and an olefinic amino acid; and
wherein X₇ is selected from the group consisting of: an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid.
6. A G protein peptidomimetic or salt thereof comprising or consisting of a sequence of the structure (XIV):

(XIV)
(SEQ ID NO: 18)
FX₂X₃X₄KDX₆ILQX₁₁NLX₁₄EYNX₁₈V

wherein X₂ is asparagine (N) or alanine (A);
wherein X₃ is selected from the group consisting of: an amino acid containing an azidated side-chain, an amino acid residue containing an alkynyl side-chain, an amino acid containing a carboxylic acid group side-chain, an amino acid containing an amine side-chain, an amino acid containing a thiol side-chain and an olefinic amino acid;
wherein X₄ is cysteine (C) or valine (V);
wherein X₇ is selected from the group consisting of: an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid;
wherein X₁₁ is methionine (M) or leucine (L);
wherein X₁₄ is arginine (R) or lysine (K); and
wherein X₁₈ is leucine (L), or an alanine analogue, phenylalanine (F), tyrosine (Y), or tryptophan (W), wherein the alanine analogue is a molecule resulting from the replacement of at least one hydrogen of an alanine by at least one moiety selected from the group comprising C₆₋₁₂ cycloalkyl, C₆₋₁₂ aryl, heteroaryl, and C₆₋₁₂ cycloalkenyl; each moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl;
wherein the peptidomimetic comprises a covalent tether formed from the reaction of the side-chain of X₃ with the side-chain of X₇, wherein X₇ is an amino acid containing an azidated side-chain and X₃ is an amino acid containing an alkynyl side-chain, wherein X₃ is an amino acid containing an azidated side-chain and X₇ is an amino acid containing an alkynyl side-chain, wherein X₃ is an amino acid containing a carboxylic acid group side-chain and X₇ is an amino acid containing an amine side-chain, wherein X₇ is an amino acid containing a carboxylic acid group side-chain and X₃ is an amino acid containing an amine side-chain, wherein X₃ and X₇ are olefinic amino acids, or wherein X₃ and X₇ are amino acids containing a thiol side-chain.
7. The G protein peptidomimetic or salt thereof according to any one of 1 to 6, wherein X₇ is an amino acid containing an azidated side-chain and X₃ is an amino acid containing an alkynyl side-chain or wherein X₃ is an amino acid containing an azidated side-chain and X₇ is an amino acid containing an alkynyl side-chain, preferably wherein X₇ is an amino acid containing an azidated side-chain and X₃ is an amino acid containing an alkynyl side-chain.
8. The G protein peptidomimetic or salt thereof according to 1 or 2, wherein the peptidomimetic comprises a covalent tether formed between X₇ and X₁₁ from the reaction of an amino acid containing an azidated side-chain with an amino acid containing an alkynyl side-chain, or from the reaction of an amino acid containing an amine side-chain with an amino acid containing a carboxylic acid group side-chain, or from the reaction between two olefinic amino acids, or from the reaction between two amino acids each containing a thiol group side-chain, wherein said covalent tether is not part of the linear peptide backbone.
9. The G protein peptidomimetic or salt thereof according to any one of 1, 2 or 8, comprising a sequence of the structure (VI) or (VII):

(VI)
(SEQ ID NO: 26)
FNDX₄KDX₇ILQX₁₁NLRX₁₅X₁₆X₁₇X₁₈X₁₉
(VII)
(SEQ ID NO: 27)
FAAVKDX₇ILQX₁₁NLKX₁₅X₁₆X₁₇X₁₈X₁₉,

wherein X₇ is selected from the group consisting of: an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid; and
wherein X₁₁ is selected from the group consisting of: an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid.
10. The G protein peptidomimetic or salt thereof according to 1 or 2, wherein the peptidomimetic comprises a covalent tether formed between X₁₁ and X₁₄ from the reaction of an amino acid containing an azidated side-chain with an amino acid containing an alkynyl side-chain, or from the reaction of an amino acid containing an amine side-chain with an amino acid containing a carboxylic acid group side-chain, or from the reaction between two olefinic amino acids, or from the reaction between two amino acids each containing a thiol group side-chain, wherein said covalent tether is not part of the linear peptide backbone.
11. The G protein peptidomimetic or salt thereof according to any one of 1, 2 or 10, comprising a sequence of the structure (VIII) or (IX):

(VIII)
(SEQ ID NO: 28)
FNDX₄KDIILQX₁₁NLX₁₄X₁₅X₁₆X₁₇X₁₈X₁₉
(IX)
(SEQ ID NO: 29)
FAAVKDTILQX₁₁NLX₁₄X₁₅X₁₆X₁₇X₁₈X₁₉,

wherein X₁₁ is selected from the group consisting of: an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid;
wherein X₁₄ is selected from the group consisting of: an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid.
12. The G protein peptidomimetic or salt thereof according to 1 or 2, wherein the peptidomimetic comprises a covalent tether formed between X₇ and X₁₄ from the reaction of an amino acid containing an azidated side-chain with an amino acid containing an alkynyl side-chain, or from the reaction of an amino acid containing an amine side-chain with an amino acid containing a carboxylic acid group side-chain, or from the reaction between two olefinic amino acids, or from the reaction between two amino acids each containing a thiol group side-chain, wherein said covalent tether is not part of the linear peptide backbone.
13. The G protein peptidomimetic or salt thereof according to any on of 1, 2 or 12, comprising a sequence of the structure (X) or (XI):

(X)
(SEQ ID NO: 30)
FNDX₄KDX₇ILQMNLX₁₄X₁₅X₁₆X₁₇X₁₈X₁₉
(XI)
(SEQ ID NO: 31)
FAAVKDX₇ILQLNLX₁₄X₁₅X₁₆X₁₇X₁₈X₁₉,

wherein X₇ is selected from the group consisting of: an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid; and
wherein X₁₄ is selected from the group consisting of: an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid.
14. The G protein peptidomimetic or salt thereof according to any one of claims 1 to 13, wherein said amino acid containing an azidated side-chain is azidolysine (Azk) and wherein said amino acid containing an alkynyl side-chain is propargylglycine (Pra).
15. The G protein peptidomimetic or salt thereof according to 1 or 2, comprising a sequence of the structure (XII) or (XIII):

(XII)
(SEQ ID NO: 32)
FNDX₄KDIILQMNLRX₁₅X₁₆X₁₇X₁₈X₁₉
(XIII)
(SEQ ID NO: 33)
FAAVKDTILQLNLKX₁₅X₁₆X₁₇X₁₈X₁₉.

16. The G protein peptidomimetic or salt thereof according to any one of 1 to 5, or 7 to 15, wherein X₁₉ is an acidic amino acid, preferably wherein X₁₉ is aspartic acid (D), glutamic acid (E), D-aspartic acid or D-glutamic acid.
17. The G protein peptidomimetic or salt thereof according to 16, wherein X₁₇ is asparagine (N), wherein X₁₆ is tyrosine (Y), wherein X₁₅ is glutamic acid (E) and wherein X₄ is cysteine (C).
18. The G protein peptidomimetic or salt thereof according to any one of 1 to 5, or 7 to 17, wherein X₁₈ is glutamic acid (E).
19. The G protein peptidomimetic or salt thereof according to 18, wherein X₁₉ is valine (V), wherein X₁₇ is asparagine (N), wherein X₁₆ is tyrosine (Y), wherein X₁₅ is glutamic acid (E) and wherein X₄ is cysteine (C).
20. The G protein peptidomimetic or salt thereof according to any one of 1 to 5, or 7 to 16, or 18, wherein X₁₇ is cysteine (C) and wherein X₄ is an amino acid without a thiol side-chain
21. The G protein peptidomimetic or salt thereof according to 20, wherein X₁₉ is valine (V), wherein X₁₆ is tyrosine (Y) and wherein X₁₅ is glutamic acid (E).
22. The G protein peptidomimetic or salt thereof according to any one of 1 to 5, or 7 to 16, 18, or 20, wherein X₁₆ is an aromatic amino acid selected from the group comprising or consisting of: Phe(4′guanidino), tryptophan (W), phenylalanine (F), naphthylalanine, 1-naphthylalanine (1-NaI) and 2-naphthylalanine (2-NaI); preferably Phe(4′guanidino).
23. The G protein peptidomimetic or salt thereof according to 22, wherein X₁₉ is valine (V), wherein X₁₇ is asparagine (N), wherein X₁₅ is glutamic acid (E) and wherein X₄ is cysteine (C).
24. The G protein peptidomimetic or salt thereof according to any one of 1 to 5, or 7 to 16, 18, 20, or 22, wherein X₁₅ is homoglutamic acid or aspartic acid, preferably homoglutamic acid.
25. The G protein peptidomimetic or salt thereof according to 24, wherein X₁₉ is valine (V), wherein X₁₇ is asparagine (N), wherein X₁₆ is tyrosine (Y), and wherein X₄ is cysteine (C).
26. The G protein peptidomimetic or salt thereof according to any one of 1 to 17, or 20 to 25, wherein X₁₈ is a moiety of formula (Ia):

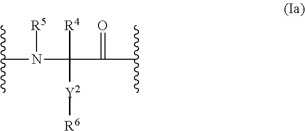

wherein:
R⁴ is hydrogen or C₁₋₆ alkyl;
R⁵ is hydrogen or C₁₋₆ alkyl;
R⁶ is a moiety selected from the group comprising C₆₋₁₂ cycloalkyl, C₆₋₁₂ aryl, heteroaryl, and C₆₋₁₂ cycloalkenyl; each moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl;
Y² is —C(R⁷)R⁸— or —C(═O)—;
R⁷ is selected from the group comprising hydrogen, OH, SH, C₁₋₆ alkyl, C₃₋₁₂ cycloalkyl, C₁₋₆ alkoxy, amino, and halo;
R⁸ is hydrogen or C₁₋₆ alkyl;
or R⁷ and at least one substituent of R⁶ together with the carbon atom to which they are attached form a C₃₋₁₂ cycloalkyl, wherein said C₃₋₁₂ cycloalkyl can be optionally substituted with one or more substituents independently selected from the group comprising C₁₋₆ alkyl, OH, halo, C₃₋₁₂ cycloalkyl, C₂₋₆ alkenyl, C₁₋₆ alkoxy, oxo, ═CH₂, amino, mono- or di-C₁₋₆ alkylamino, amino C₁₋₆ alkyl, and haloC₁₋₆ alkyl, or two substituents together with the atom to which they are attached may form a C₃₋₁₂ cycloalkyl, a C₅₋₁₂ cycloalkenyl, a heterocycloalkyl or an C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl.
27. The G protein peptidomimetic or salt thereof according to any one of 1 to 17, or 20 to 26, wherein X₁₈ is a moiety of formula (Ic):

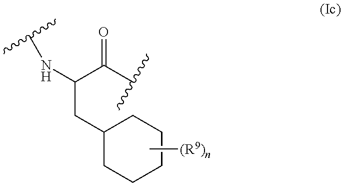

wherein
n is an integer selected from 0, 1, 2, 3, 4, or 5;
R⁹ is selected from the group comprising OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or two R⁹ together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl.
28. The G protein peptidomimetic or salt thereof according to any one of 1 to 17, or 20 to 27, wherein X₁₈ is cyclohexylalanine (Cha).
29. The G protein peptidomimetic or salt thereof according to any one of 1 to 17, or 20 to 26, wherein X₁₈ is phenylalanine (F), tyrosine (Y), or tryptophan (W).
30. The G protein peptidomimetic or salt thereof according to any one of 1 to 17, or 20 to 25, wherein X₁₈ is leucine (L).
31. The G protein peptidomimetic or salt thereof according to any one of 1 to 7, 14, 18, 19, or 26 to 30, comprising a sequence of the structure (XV) or (XVI):

(XV)
(SEQ ID NO: 19)
FNX₃CKDX₇ILQMNLREYNX₁₈V
(XVI)
(SEQ ID NO: 20)
FAX₃VKDX₇ILQLNLKEYNX₁₈V.

32. The G protein peptidomimetic or salt thereof according to any one of 1 to 31, further comprising at least one basic amino acid at its N-terminus, preferably from 1 to 10 basic amino acids, more preferably from 1 to 8 basic amino acids, even more preferably from 3 to 8 basic amino acids.
33. The G protein peptidomimetic or salt thereof according to 32, wherein the basic amino acid is selected from lysine (K), histidine (H), arginine (R) and D-arginine.
34. The G protein peptidomimetic or salt thereof according to any one of 1 to 33, wherein said peptidomimetic comprises a triple lysine (K) at its N-terminus.
35. The G protein peptidomimetic or salt thereof according to any one of 1 to 31, further comprising a cell-penetrating peptide (CPP) at its N-terminus, preferably a cationic CPP or an amphipathic CPP such as a CPP selected from the group consisting of: RW9 consisting of the sequence set forth in SEQ ID NO:39, Arg₈ consisting of the sequence set forth in SEQ ID NO:36 and Arg₄ consisting of the sequence set forth in SEQ ID NO:37.
36. The G protein peptidomimetic or salt thereof according to any one of 1 to 35, wherein said peptidomimetic comprises an N-terminal modification such as an N-terminal modification selected from an N-terminal acylation, an N-terminal acetylation, or an N-terminal alkylation.
37. The G protein peptidomimetic or salt thereof according to any one of 1 to 36, wherein said peptidomimetic comprises an N-terminal acetylation.
38. The G protein peptidomimetic or salt thereof according to any one of 1 to 7, 14, 15, 26 to 28, 30 to 34, 36, or 37, wherein said G protein peptidomimetic is selected from Table C.
39. The G protein peptidomimetic or salt thereof according to any one of 1 to 38, wherein said G protein peptidomimetic is capable of stabilizing a G protein-coupled receptor (GPCR) in an active conformational state, wherein said GPCR is preferably a G_{q/11} protein-coupled receptor, more preferably muscarinic acetylcholine receptor 1 (M1R) or ghrelin receptor (GHSR).
40. The G protein peptidomimetic or salt thereof according to any one of 1 to 7, 14, 26 to 28, 30 to 34, or 36 to 39, which is compound SBL-GQ-05 as defined by SEQ ID NO:5, compound SBL-GQ-06 as defined by SEQ ID NO:6, compound SBL-GQ-11 as defined by SEQ ID NO:11, compound SBL-GQ-12 as defined by SEQ ID NO:12.
41. The G protein peptidomimetic or salt thereof according to any one of 1 to 7, 14, 30 to 36, or 39, which is compound SBL-GQ-13 as defined by SEQ ID NO:41, compound SBL-GQ-14 as defined by SEQ ID NO:42, compound SBL-GQ-15 as defined by SEQ ID NO:43, or compound SBL-GQ-25 as defined by SEQ ID NO:51.
42. A fusion polypeptide comprising a G protein peptidomimetic according to any one of 1 to 41 and a GPCR, wherein said G protein peptidomimetic and GPCR are optionally fused through a linker.
43. A complex comprising a G protein peptidomimetic according to any one of 1 to 41 and a GPCR.
44. The complex according to 43 further comprising a receptor ligand.
45. A composition comprising a fusion polypeptide according to 42 or a complex according to 43 or 44.
46. Use, preferably in vitro use, of a G protein peptidomimetic according to any one of 1 to 41, a fusion polypeptide according to 42, a complex according to 43 or 44, or a composition according to 45 to capture a GPCR in an active conformation.
47. A method of capturing a GPCR in an active conformation, said method comprising the steps of:
a) bringing a G protein peptidomimetic according to any one of 1 to 41 into contact with a GPCR, and
b) allowing the G protein peptidomimetic to bind to the GPCR, whereby the GPCR is captured in an active conformation.
48. Use of a G protein peptidomimetic according to any one of 1 to 41 for crystallizing a complex of the G protein peptidomimetic and a GPCR and optionally a ligand of the GPCR.
49. A method of crystallizing a complex of a G protein peptidomimetic according to any one of 1 to 41 and a GPCR and optionally a ligand of the GPCR, the method comprising the steps of:
   a) providing a G protein peptidomimetic according to any one of 1 to 41 and a GPCR, and optionally a ligand of the GPCR,
   b) allowing the formation of a complex of the G protein peptidomimetic, the GPCR and optionally the ligand, and
   c) crystallizing said complex of step b) to form a crystal.
50. A method of determining the crystal structure of a GPCR in an active conformation, the method comprising the steps of:
   a. crystallizing a complex of a G protein peptidomimetic according to any one of 1 to 41 and a GPCR, and optionally a ligand of the GPCR according to the method defined in 49 to form a crystal, and
   b. obtaining the atomic coordinates of the crystal.
51. Use of a G protein peptidomimetic according to any one of 1 to 41, a complex according to 43 or 44, a fusion polypeptide according to 42, or a composition according to 45 for identifying compounds that are capable of interacting with the GPCR, preferably active conformation-selective ligands of the GPCR.
52. A screening method for identifying compounds capable of interacting with a GPCR, preferably active conformation-selective ligands of the GPCR, the method comprising the steps:
a) contacting the GPCR with a test compound and a G protein peptidomimetic according to any one of 1 to 41, a complex according to 43 or 44, a fusion polypeptide according to 42, or a composition according to 45;
b) evaluating binding of the test compound to the GPCR; and
c) optionally selecting a test compound that binds to the GPCR as a compound capable of interacting with the GPCR.
53. Use, preferably in vitro use, of a G protein peptidomimetic according to any one of 1 to 41 for allosterically modulating a GPCR.
54. Use, preferably in vitro use, of a G protein peptidomimetic according to any one of 1 to 41 as a biosensor, in particular a biosensor to detect conformational change of a GPCR, a biosensor to assess the localization and/or trafficking of a GPCR, and/or a biosensor to investigate a GPCR signalling pathway.

### BRIEF DESCRIPTION OF THE FIGURES

The teaching of the application is illustrated by the following Figures which are to be considered as illustrative only and do not in any way limit the scope of the claims.

FIG. 1: General scheme of a Fmoc-based solid phase peptide synthesis. RAA: side chain of amino acid; X═—O— for Wang resin; X═—NH— for Rink Amide resin.

FIG. 2: Cryo-EM structure of the muscarinic acetylcholine 1 receptor (M1R) in complex with Gα₁₁. Zoom on the interaction (depicted in dotted line) of the penultimate L358 with residues in the TM5 and TM6 of the receptor.

FIG. 3: Cryo-EM structure of the 5-HT_{2A} receptor in complex with mini-G_{q}. Zoom on the interaction (depicted in dotted lines) of the penultimate L245 with residues in the TM3 and TM6 of the receptor.

FIGS. 4A-4B: Radioligand assay to quantify stabilization of the G_{q/11}-coupled receptor (M1R) in the active conformation. (FIG. 4A) Screening of the linear (mini-)G_{q/11} mimetics, at 100 μM (in duplicate). (FIG. 4B) Screening of the triazole stapled (mini-)G_{q/11} mimetics, at 100 μM (in duplicate).

FIGS. 5A-5B: Bimane fluorescence assay on the ghrelin receptor (GHSR). (a) Representation of GHSR with a monobromobimane attached to a Cys residue, at the cytoplasmatic end of TM6. (b) Maximum emission wavelength of the bimane fluorophore recorded for the different (mini-)G_{q/11} mimetics in presence and absence of the full agonist JMV1843. The assay was also performed with and without the G_{q} protein as reference. The data represent the mean±standard deviation of three different experiments.

FIG. 6: Effect of the stapled G_{q} peptidomimetics on the emission properties of bimane attached to the ghrelin receptor in the presence of the full agonist JMV1843. The data represent the mean±standard deviation of three different experiments.

FIG. 7: Schematic representation of the IP-One G_{q} assay.

FIG. 8: IP-One G_{q} assay targeting the ghrelin receptor in the presence of the indicated G_{q/11} peptidomimetics at 10 μM and varying concentrations of the agonist. Assays were performed three times (n=3), in triplicate. Fluorescence ratio (665 nm/620 nm) (HTRF ratio) is shown.

FIG. 9: IP-One G_{q} assay targeting the ghrelin receptor in the presence of the indicated G_{q/11} peptidomimetics at 5 or 10 μM and varying concentrations of the agonist. Assays were performed two times (n=2), in triplicate. Fluorescence ratio (665 nm/620 nm) is shown.

FIG. 10: Dose-response curve of the G_{q/11} peptidomimetics in the IP-One G_{q} assay on HEK293 cells overexpressing the ghrelin receptor. Assay was performed two times (n=2), in triplicate.

FIG. 11: Cell internalization assay with HEK293 cells overexpressing the ghrelin receptor. The cells were incubated with 5 and 10 μM of the indicated SulfoCy5-labeled G_{q/11} peptidomimetics for 3 h 45 min, at 37° C. After washing the cells with PBS, fluorescence was measured and autofluorescence of the cells (measured in cells with no peptide incubation) was subtracted to obtain a calculated fluorescence. The calculated fluorescence is shown as the product of 3 separate experiments, each carried out in triplicate.

FIGS. 12A-12C: Cytotoxicity assay. HEK293 1C8 cells were incubated with varying concentrations of the indicated G_{q/11} peptidomimetics for 3 h 45 min at 37° C. to evaluate the cytotoxicity of the G_{q/11} peptidomimetics at different concentrations.

### DETAILED DESCRIPTION OF THE INVENTION

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. Where reference is made to embodiments as comprising certain elements or steps, this encompasses also embodiments which consist essentially of the recited elements or steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

As used herein, the term “and/or” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, aspects, structures or characteristics may be combined in any suitable manner, as would be apparent to one of ordinary skill in the art from this disclosure, in one or more embodiments. Also embodiments described for an aspect of the invention may be used for another aspect of the invention and can be combined.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects.

In each of the following definitions, the number of carbon atoms represents the maximum number of carbon atoms generally optimally present in the moiety, group, substituent or linker; it is understood that where otherwise indicated in the present application, the number of carbon atoms represents the optimal maximum number of carbon atoms for that particular moiety, group, substituent or linker.

Whenever the term “substituted” is used herein, it is meant to indicate that one or more hydrogen atoms on the atom indicated in the expression using “substituted” is replaced with a selection from the indicated group, provided that the indicated atom's normal valence is not exceeded, and that the substitution results in a chemically stable compound, i.e. a compound that is sufficiently robust to survive isolation from a reaction mixture.

The term “halo” or “halogen” as a group or part of a group is generic for fluoro, chloro, bromo, iodo.

The term “oxo” as used herein means the ═O group.

The term “amino” as used herein means the —NH₂ group.

The term “azido” refers to the —N═N═N group.

The term “azidated” refers to a compound comprising an azido group.

The term “alkyl”, as a group or part of a group, refers to normal, secondary, or tertiary, linear, branched or straight hydrocarbon with no site of unsaturation of formula CₙH₂ₙ₊₁ wherein n is preferably a number ranging from 1 to 6. Thus, for example, “C₁₋₆ alkyl” includes all linear or branched alkyl groups with between 1 and 6 carbon atoms, and thus includes methyl, ethyl, 1-propyl (n-propyl), 2-propyl (iPr), 1-butyl, 2-methyl-1-propyl (i-Bu), 2-butyl (s-Bu), 2-dimethyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl. For example, “C₁₋₄ alkyl” includes all linear or branched alkyl groups with between 1 and 5 carbon atoms, and thus includes methyl, ethyl, n-propyl, i-propyl, butyl and its isomers (e.g. n-butyl, i-butyl and t-butyl); pentyl and its isomers. For example, “C₁₋₄ alkyl” includes all linear or branched alkyl groups with between 1 and 4 carbon atoms, and thus includes methyl, ethyl, n-propyl, i-propyl, butyl and its isomers (e.g. n-butyl, i-butyl and t-butyl). For example, “C₁₋₃ alkyl” includes all linear or branched alkyl groups with between 1 and 3 carbon atoms, and thus includes methyl, ethyl, n-propyl, i-propyl.

The term “haloC₁₋₆ alkyl” as a group or part of a group, refers to a C₁₋₆ alkyl group having the meaning as defined above wherein one or more hydrogen atoms are each replaced with one or more halogen as defined herein. Non-limiting examples of such haloC₁₋₆ alkyl groups include chloromethyl, 1-bromoethyl, fluoromethyl, difluoromethyl, trifluoromethyl, 1,1,1-trifluoroethyl and the like.

The term “C₁₋₆ alkoxy”, as a group or part of a group, refers to a group having the formula —OR^{a} wherein R^{a} is C₁₋₆ alkyl as defined herein above. Non-limiting examples of suitable C₁₋₆ alkoxy include methoxy, ethoxy, propoxy, isopropoxy, butoxy, isobutoxy, sec-butoxy, tert-butoxy, pentyloxy and hexyloxy.

The term “C₂₋₆ alkenyl” as a group or part of a group, refers to an unsaturated hydrocarbyl group, which may be linear, or branched, comprising one or more carbon-carbon double bonds, and comprising from 2 to 6 carbon atoms. Examples of C₂₋₆ alkenyl groups are ethenyl, 2-propenyl, 2-butenyl, 3-butenyl, 2-pentenyl and its isomers, 2-hexenyl and its isomers, 2,4-pentadienyl, and the like.

The term “alkynyl” or as used herein refers to C₂₋₆ normal, secondary, tertiary, linear, branched or straight hydrocarbon with at least one site (usually 1 to 3, preferably 1) of unsaturation, namely a carbon-carbon, sp triple bond. Examples include, but are not limited to: ethynyl (—C≡CH), 3-ethyl-cyclohept-1-ynylene, and 1-propynyl (propargyl, —CH₂C≡CH).

The term “C₃₋₁₂ cycloalkyl”, as a group or part of a group, refers to a cyclic alkyl group, that is a monovalent, saturated, hydrocarbyl group having 1 or more cyclic structure, and comprising from 3 to 12 carbon atoms, preferably from 5 to 6 carbon atoms. Cycloalkyl includes all saturated hydrocarbon groups containing one or more rings, including monocyclic or bicyclic groups. The further rings of multi-ring cycloalkyls may be either fused, bridged and/or joined through one or more spiro atoms. Examples of C₃₋₁₂ cycloalkyl include by are not limited to such instance cyclopropyl, cyclobutyl, cyclopentyl, cyclopropylethylene, methylcyclopropylene, cyclohexyl, cycloheptyl, cyclooctyl, cyclooctylmethylene, norbornyl, fenchyl, trimethyltricycloheptyl, decalinyl, adamantyl and the like. Examples of C₃₋₆ cycloalkyl groups include but are not limited to cyclopropyl, cyclobutyl, cyclopentyl, and cyclohexyl.

The term “cycloalkenyl” as used herein refers to a non-aromatic hydrocarbon group having from 5 to 12 carbon atoms with at least one site (usually 1 to 3, preferably 1) of unsaturation, namely a carbon-carbon, sp2 double bond and consisting of or comprising a C₅₋₁₀ monocyclic or C₇₋₁₂ polycyclic hydrocarbon.

Examples include, but are not limited to: cyclopentenyl (—C₅H₇), cyclopentenylpropylene, methylcyclohexenylene and cyclohexenyl (—C₆H₉). The double bond may be in the cis or trans configuration. In particular embodiments, the term cycloalkenyl refers to C₅₋₁₂ cycloalkenyl (cyclic C₅₋₁₂ hydrocarbons), yet more in particular to C₆₋₁₂ cycloalkenyl (cyclic C₆₋₁₂ hydrocarbons), still more in particular to C₆₋₁₀ cycloalkenyl (cyclic C₆₋₁₀ hydrocarbons) as further defined herein above with at least one site of unsaturation, namely a carbon-carbon, sp2 double bond.

The term “C₆₋₁₂ aryl”, as a group or part of a group, refers to a polyunsaturated, aromatic hydrocarbyl group having a single ring (i.e. phenyl) or multiple aromatic rings fused together (e.g. naphthyl), or linked covalently, typically comprising 6 to 12 carbon atoms; wherein at least one ring is aromatic, preferably comprising 6 to 10 carbon atoms, wherein at least one ring is aromatic. The aromatic ring may optionally include one to two additional rings (either cycloalkyl, heterocyclyl or heteroaryl) fused thereto. Examples of suitable aryl include C₆₋₁₀ aryl, more preferably C₆₋₈ aryl. Non-limiting examples of C₆₋₁₂ aryl comprise phenyl, biphenylyl, biphenylenyl, or 1- or 2-naphthalenyl; 5- or 6-tetralinyl, 1-, 2-, 3-, 4-, 5-, 6-, 7- or 8-azulenyl, 4-, 5-, 6 or 7-indenyl, 4- or 5-indanyl, 5-, 6-, 7- or 8-tetrahydronaphthyl, 1,2,3,4-tetrahydronaphthyl, and 1,4-dihydronaphthyl; 1-, 2-, 3-, 4- or 5-pyrenyl.

The term “heteroaryl” refers but is not limited to an aromatic ring system of 5 to 12 atoms including at least one N, O, S, or P, containing 1 or 2 rings which can be fused together or linked covalently, each ring typically containing 5 to 6 atoms; at least one of said ring is aromatic, where the N and S heteroatoms may optionally be oxidized and the N heteroatoms may optionally be quaternized, and wherein at least one carbon atom of said heteroaryl can be oxidized to form at least one C═O. Such rings may be fused to an aryl, cycloalkyl, heteroaryl or heterocyclyl ring. Non-limiting examples of such heteroaryl, include: triazol-2-yl, pyridinyl, 1H-pyrazol-5-yl, pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, benzo[d]oxazol-2(3H)-one, 2,3-dihydro-benzofuranyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl; preferably said heteroaryl group is selected from the group comprising pyridyl, 1,3-benzodioxolyl, benzo[d]oxazol-2(3H)-one, 2,3-dihydro-benzofuranyl, pyrazinyl, pyrazolyl, pyrrolyl, isoxazolyl, thiophenyl, imidazolyl, benzimidazolyl, pyrimidinyl, s-triazinyl, oxazolyl, isothiazolyl, furyl, thienyl, triazolyl and thiazolyl.

The term “heterocycloalkyl” as used herein refer to non-aromatic, fully saturated ring system of 3 to 12 atoms, comprising at least two ring forming carbon atoms and at least one ring forming heteroatom such as at least one N, O, or S, (for example, 3 to 7 member monocyclic, 7 to 11 member bicyclic, or comprising a total of 3 to 10 ring atoms) wherein at least one ring is a heterocycloalkyl and wherein said ring may be fused to an aryl, cycloalkyl, heteroaryl or heterocycloalkyl ring. Each ring of the heterocyclyl may have 1, 2, 3 or 4 heteroatoms selected from N, O and/or S, where the N and S heteroatoms may optionally be oxidized and the N heteroatoms may optionally be quaternized; and wherein at least one carbon atom of heterocyclyl can be oxidized to form at least one C═O. The heterocyclic may be attached at any heteroatom or carbon atom of the ring or ring system, where valence allows. The rings of multi-ring heterocyclyls may be fused, bridged and/or joined through one or more spiro atoms. Suitable heterocycloalkyl groups include oxetanyl, azetidinyl, tetrahydrofuranyl, dioxolanyl, pyrrolidinyl, oxazolidinyl, thiazolidinyl, isothiazolidinyl, imidazolidinyl, tetrahydropyranyl, tetrahydrothiopyranyl, piperidinyl, piperazinyl, morpholinyl, thiomorpholinyl, azepanyl, oxazepanyl, diazepanyl, thiadiazepanyl and azocanyl.

The term “mono- or di-C₁₋₆ alkylamino”, as a group or part of a group, refers to a group of formula —N(R′)(R^{b}) wherein R^{b} is hydrogen, or C₁₋₆ alkyl, R^{a} is C₁₋₆ alkyl. Thus, alkylamino include mono-alkyl amino group (e.g. mono-alkylamino group such as methylamino and ethylamino), and di-alkylamino group (e.g. di-alkylamino group such as dimethylamino and diethylamino). Non-limiting examples of suitable mono- or di-alkylamino groups include n-propylamino, isopropylamino, n-butylamino, i-butylamino, sec-butylamino, t-butylamino, pentylamino, n-hexylamino, di-n-propylamino, di-i-propylamino, ethylmethylamino, methyl-n-propylamino, methyl-1-propylamino, n-butylmethylamino, i-butylmethylamino, t-butylmethylamino, ethyl-n-propylamino, ethyl-1-propylamino, n-butylethylamino, i-butylethylamino, t-butylethylamino, di-n-butylamino, di-i-butylamino, methylpentylamino, methylhexylamino, ethylpentylamine, ethylhexylamino, propylpentylamino, propylhexylamino, and the like.

The term “Pra” as used herein refers to moiety of formula

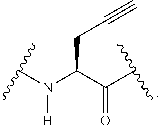 [SMILES: *N[C@@H](CC#C)C(*)=O]

The term “Azk” as used herein refers to moiety of formula

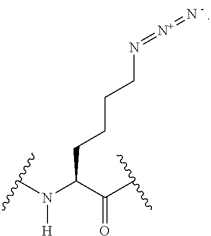 [SMILES: *N[C@@H](CCCCN=[N+]=N)C(*)=O]

The term “S5” as used herein refers to moiety of formula

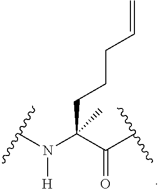 [SMILES: *N[C@@](C)(CCCC=C)C(*)=O]

The term “R5” as used herein refers to moiety of formula

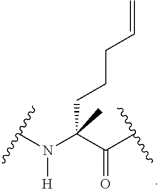 [SMILES: *N[C@](C)(CCCC=C)C(*)=O]

The term “R8” as used herein refers to moiety of formula

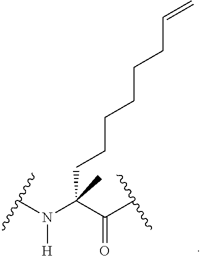 [SMILES: *N[C@](C)(CCCCCCC=C)C(*)=O]

The term “hGlu” as used herein refers to homoglutamic acid moiety of formula

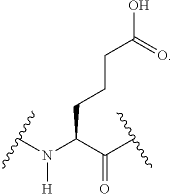 [SMILES: *N[C@@H](CCCC(=O)O)C(*)=O]

The term “Cha” as used herein refers to cyclohexylalanine moiety of formula

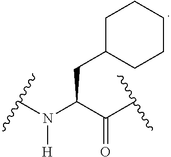 [SMILES: *N[C@@H](CC1CCCCC1)C(*)=O]

The term “Phe(4′guanidino)” as used herein refers to 4′-guanidinophenylalanine moiety of formula

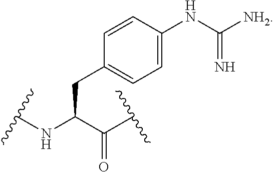 [SMILES: *N[C@@H](Cc1ccc(NC(=N)N)cc1)C(*)=O]

The term “1-NaI” as used herein refers to 1-naphthylalanine moiety of formula

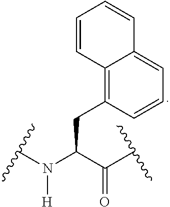 [SMILES: *N[C@@H](Cc1cccc2ccccc12)C(*)=O]

As used herein, the terms “1-naphthylalanine”, “1-naphthalanine”, and “1-naphthyl-L-alanine” are synonymous and used interchangeably.

The term “2-NaI” as used herein refers to 2-naphthylalanine moiety of formula

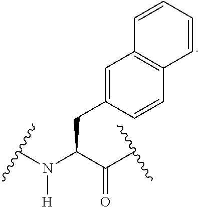 [SMILES: *N[C@@H](Cc1ccc2ccccc2c1)C(*)=O]

As used herein, the terms “2-naphthylalanine”, “2-naphthalanine”, and “2-naphthyl-L-alanine” are synonymous and used interchangeably.

Whenever used herein the term “G protein peptidomimetics” or “G_{q/11} protein peptidomimetics” or a similar term is meant to include the compounds of the general formula disclosed therein and any subgroup thereof, including all polymorphs and crystal habits thereof, and isomers thereof (including optical, geometric and tautomeric isomers) as hereinafter defined.

As used herein and unless otherwise stated, the term “stereoisomer” refers to all possible different isomeric as well as conformational forms which the “peptidomimetics” herein may possess, in particular all possible stereochemically and conformationally isomeric forms, all diastereomers, enantiomers and/or conformers of the basic molecular structure. Some compounds of the present invention may exist in different tautomeric forms, all of the latter being included within the scope of the present invention.

All documents cited in the present specification are hereby incorporated by reference in their entirety.

Preferred aspects, statements (features) and embodiments of this invention are set herein below. Each aspects, statements and embodiments of the invention so defined may be combined with any other aspects, statement and/or embodiments unless clearly indicated to the contrary. In particular, any feature indicated as being preferred or advantageous may be combined with any other feature or features or statements or aspects indicated as being preferred or advantageous.

The term “peptidomimetic” generally refers to any compound that biologically mimics a peptide or protein. Therefore, a suitable definition of a peptidomimetic as described herein may be ‘compounds whose essential elements mimic a natural peptide or protein in 3D space and which retain the ability to interact with the biological target and produce the same biological effect’ as formulated by Vagner et al. (2008, Current Opinion in Chemical Biology 292:296). A skilled person readily appreciates that peptidomimetics are commonly designed by modification of an existing peptide, although this is not a prerequisite. Thus, the design process of a peptidomimetic is not particularly limited, and may therefore be generated by various strategies including but by no means limited to approaches such as rational engineering, directed evolution, random mutagenesis, (alanine or D-amino acid) scanning approaches, or any combination thereof.

By means of guidance, the G protein peptidomimetics, in particular the G_{q/11} protein peptidomimetics, as described herein would generally be considered a type I or type II mimetic when using the classification system of Ripka and Rich (2008 Current Opinion in Chemical Biology 2:441:452). Therefore, in preferred embodiments, the G protein peptidomimetics, in particular the G_{q/11} protein peptidomimetics, as described herein are type I (i.e. structural) mimetics or type II (i.e. functional) mimetics. A skilled person appreciates from the cited art that type I mimetics show a strict analogy with the native substrate and carry all the functionalities in the same spatial orientation. Type II mimetics do not show apparent structural analogies with the native substrate, but are able to mimic its function by interacting similarly with the target receptor or enzyme. In an alternative embodiment, the G protein peptidomimetic, in particular, the G_{q/11} protein peptidomimetic may be a type II (functional-structural) mimetic that possesses a scaffold significantly different from the native substrate while displaying the interacting elements in the same spatial orientation.

More recently, a new classification system for peptidomimetics has been formulated by Pelay-Gimeno et al. (2015 Angewandte Chemie International Edition 54:8896:8927). This classification system differs from the one of Ripka and Rich in that it is centered around the degree of peptide character. When using this classification system, peptidomimetics may be stratified in four classes (A-D):
Class A mimetics contain a limited number of local modifications, which are mainly introduced to stabilize the conformation and/or limit the proteolysis degradation rate. The backbone and side-chains of the mimetics show a close alignment with the topography of the native peptide.
Class B mimetics contain more extensive modifications in their sequence, said modifications being present in both the backbone and side-chains. Non-natural amino acids are envisaged, as well as isolated small-molecule building blocks and backbone mimetics.
Class C mimetics have an increased small-molecule character when compared to class A and class B peptidomimetics and are characterized by a non-peptide unnatural frame replacing the backbone of the native substrate. The interacting elements are still presented in the same topological manner, but the peptide backbone is globally altered.
Class D mimetics mimic the mode of action of the natural substrate but do no longer share a direct link to the side-chain functionalities. Class D mimetics are considered the least similar to the original peptide.

In the present disclosure, the G protein peptidomimetics, in particular the G_{q/11} protein peptidomimetics, as described herein are generally considered to have a peptide or peptide-like backbone structure and would therefore classify as either a class A peptidomimetic or class B peptidomimetic. It is therefore understood that the G protein peptidomimetics, in particular the G_{q/11} protein peptidomimetics, as described herein still have a certain degree of sequence similarity to the native substrate, herein the α₅ helix of the Gα_{q/11} protein or mini-G_{q} protein unless explicitly indicated otherwise. Hence, in certain preferred embodiments, the G protein peptidomimetic, in particular the G_{q/11} protein peptidomimetic, is a class A peptidomimetic. In alternative embodiments, the G protein peptidomimetic, in particular the G_{q/11} protein peptidomimetic, is a class B peptidomimetic.

The term “protein” as used throughout this specification generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression-type modifications of the polypeptide chain(s), such as, without limitation, glycosylation, acetylation, guanidinylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native proteins, such as, e.g., amino acid deletions, additions and/or substitutions. The term contemplates both full-length proteins and protein parts or fragments, e.g., naturally-occurring protein parts that ensue from processing of such full-length proteins.

The term “polypeptide” as used throughout this specification generally encompasses polymeric chains of amino acid residues linked by peptide bonds. Hence, especially when a protein is only composed of a single polypeptide chain, the terms “protein” and “polypeptide” may be used interchangeably herein to denote such a protein. The term is not limited to any minimum length of the polypeptide chain. The term may encompass naturally, recombinantly, semi-synthetically or synthetically produced polypeptides. The term also encompasses polypeptides that carry one or more co- or post-expression-type modifications of the polypeptide chain, such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes polypeptide variants or mutants which carry amino acid sequence variations vis-à-vis a corresponding native polypeptide, such as, e.g., amino acid deletions, additions and/or substitutions. The term contemplates both full-length polypeptides and polypeptide parts or fragments, e.g., naturally-occurring polypeptide parts that ensue from processing of such full-length polypeptides.

The term “peptide” as used throughout this specification preferably refers to a short chain of amino acid residues linked by peptide bonds comprising 50 amino acids or less, e.g., 45 amino acids or less, preferably 40 amino acids or less, e.g., 35 amino acids or less, more preferably 30 amino acids or less, e.g., 25 amino acids or less. No strict maximal length is attributed to a peptide to still be considered a peptide. The term peptide may encompass naturally, recombinantly, semi-synthetically or synthetically produced peptides such as discussed for polypeptides above.

The term “amino acid” encompasses naturally occurring amino acids, naturally encoded amino acids or proteinogenic amino acids, non-naturally encoded amino acids, non-naturally occurring amino acids, amino acid analogues and amino acid mimetics that function in a manner similar to the naturally occurring amino acids, all in their D- and L-stereoisomers, provided their structure allows such stereoisomeric forms. The term “amino acid” as used herein also denotes an individual amino acid in a sequence or an “amino acid residue”. Amino acids are referred to herein by either their name, their commonly known three letter codes or by the one-letter codes recommended by the IUPAC-IUB Biochemical Nomenclature Commission. A “naturally encoded amino acid” refers to an amino acid that is one of the 20 common amino acids or pyrrolysine, pyrroline-carboxy-lysine or selenocysteine. The 20 common amino acids are: alanine (A or Ala), cysteine (C or Cys), aspartic acid (D or Asp), glutamic acid (E or Glu), phenylalanine (F or Phe), glycine (G or Gly), histidine (H or His), isoleucine (I or lie), lysine (K or Lys), leucine (L or Leu), methionine (M or Met), asparagine (N or Asn), proline (P or Pro), glutamine (Q or Gln), arginine (R or Arg), serine (S or Ser), threonine (T or Thr), valine (V or Val), tryptophan (W or Trp), and tyrosine (Y or Tyr). Also included are amino acid analogues, in which one or more individual atoms have been replaced either with a different atom, an isotope of the same atom, or with a different functional group.

“Side-chain” as used herein and spelled interchangeably in the art by “side chain” or “sidechain” refers to a chemical group that is attached to a main chain or backbone of a molecule. Side-chain as used herein is to be interpreted in accordance with this definition unless specified otherwise. Side-chains of amino acids are attached to the alpha-carbon of the amide backbone. Certain side-chains or groups of side-chain may be annotated or simplified in the art by the letter “R”. Amino acid side-chains determine both charge and polarity of amino acids.

The terms “backbone”, “(poly)peptide backbone”, or “protein backbone” as used interchangeably herein are to be interpreted in their generally accepted meaning in the art. The peptide backbone is thus indicative for the peptide bonds between a first amino acid to a second consecutive amino acid. Peptide bonds are thus amide bonds that link the non-side-chain or alpha-carboxyl group of one amino acid with the non-side chain or alpha-amino group of the other amino acid. Peptide bond formation is a dehydration synthesis reaction.

In accordance with the above, the term peptidomimetic as used herein is used to describe peptide or peptide-like molecules that do not have a 100% sequence identity to the naturally occurring substrate peptide or protein yet nevertheless exert a similar or identical function to said peptide or protein. Hence, the sequence of a peptidomimetic does not occur in natural peptides or proteins, but contains at least one residue that has been substituted, chemically modified, deleted, and/or added when compared to the naturally occurring sequence. A peptidomimetic may therefore comprise one or more mutated amino acids and/or one or more non-naturally occurring (i.e. artificial) amino acids as part of its protein or protein-like chain compared to the native substrate peptide. Optionally, the peptidomimetics as described herein may have a higher stability towards proteolysis, better permeability properties, better transport properties, and/or improved selectivity against non-target receptors compared to the naturally occurring peptide. It is evident that many of the herein described mutations and modifications may be replaced by amino acid analogues known to a skilled person. Peptidomimetic molecules comprising one or more of such amino acid analogues are also envisaged by the inventors.

The peptidomimetics disclosed herein can be readily prepared using standard techniques known in the art, including chemical synthesis (Merrifield, 1963) and genetic engineering. When non-proteinogenic amino acids are contained in the peptidomimetics disclosed herein, they may be either added directly to the growing chain during peptide synthesis or prepared by chemical modification of the complete synthesized peptide, depending on the nature of the desired non-proteinogenic amino acid. Those of skill in the chemical synthesis art are well aware of which non-proteinogenic amino acids may be added directly and which must be synthesized by chemically modifying the complete peptide chain following peptide synthesis (reviewed in Jaradat 2018 Amino Acids 50:39-68). Alternatively, where the peptidomimetic is synthesized by a cellular expression system, certain codons may be reprogrammed and allocated in said expression system to encode non-naturally occurring amino acids (see e.g. Xie and Schultz 2005 Current Opinion Chemical Biology 548:554 and Kuo et al. 2018 Current Genetics 327-333). The occurrence of non-naturally occurring amino acids in the peptidomimetics therefore does not exclude synthesis by expression systems.

The term “G protein peptidomimetic” as used herein refers to a compound that biologically mimics a G protein, in particular the α-subunit of a G protein. In particular, the G protein peptidomimetics disclosed herein produce and/or stabilize a conformational change of a GPCR upon binding or interaction with the GPCR, which mimics the conformational state of the GPCR upon interaction or binding with the G protein. It is understood that “a G protein” is not to be regarded in a limiting singular “G protein” interpretation, and a single G protein peptidomimetic may therefore biologically mimic one or more different (α-subunits of) G proteins.

With “G proteins” are meant the family of guanine nucleotide-binding proteins involved in transmitting chemical signals outside the cell and causing changes inside the cell. G proteins are key molecular components in the intracellular signal transduction following ligand binding to the extracellular domain of a GPCR. They are also referred to as “heterotrimeric G proteins”, or “large G proteins”. G proteins consist of three subunits: alpha (α), beta (β), and gamma (γ) and their classification is largely based on the identity of their distinct a subunits, and the nature of the subsequent transduction event. Further classification of G proteins has come from cDNA sequence homology analysis. G proteins bind either guanosine diphosphate (GDP) or guanosine triphosphate (GTP) and possess highly homologous guanine nucleotide binding domains and distinct domains for interactions with receptors and effectors. Different subclasses of Gα proteins, such as Gαs, Gαi, Gαq and Gα12, amongst others, signal through distinct pathways involving second messenger molecules such as cAMP, inositol triphosphate (IP3), diacylglycerol, intracellular Ca²⁺ and RhoA GTPases. To illustrate this further, the α subunit (39-46 kDa) contains the guanine nucleotide binding site and possesses GTPase activity; the β(37 kDa) and γ (8 kDa) subunits are tightly associated and function as a βγ heterodimer. There are 23 types (including some splicing isoforms) of α subunits, 6 of β, and 11 of γ currently described. The classes of G protein and subunits are subscripted: thus, for example, the α subunit of Gₛ protein (which activates adenylate cyclase) is Gsα; other G proteins include Gi, which differs from Gₛ structurally (different type of a subunit) and inhibits adenylate cyclase. Further examples are provided in Table A.

TABLE A
Non-limiting examples of G proteins and their relationship
with G protein-coupled receptors and signalling pathways.

| | | Effectors/Signalling | |
|---|---|---|---|
| G protein family | α subunit | pathways | Use/Receptors |
| Gi family | | | |
| Gi | αi | Inhibition of adenylate | Acetylcholine M2 & M4 receptors |
| Go | αo | cyclase (cAMP ↓) | Adenosine A1 & A3 receptors |
| | | Closing Ca²⁺ channels | Adrenergic α2A, α2B, & α2C receptors |
| | | (Ca²⁺ ↓) | Apelin receptors |
| | | | Calcium-sensing receptor |
| | | | Chemokine CXCR4 receptor |
| | | | Dopamine D2, D3, D4 |
| | | | GABAB receptor |
| | | | Glutamate mGluR2, mGluR3, mGluR4, |
| | | | mGluR6, mGluR7, & mGluR8 receptors |
| | | | Histamine H2 & H3 & H4 receptors |
| | | | Melatonin MT1, MT2, & MT3 receptors |
| | | | Muscarinic M2 & M4 receptors |
| | | | Opioid δ, κ, μ, & nociceptin receptors |
| | | | Prostaglandin EP1, EP3, FP, & TP receptors |
| | | | Serotonin 5-HT1 & 5-HT5 receptors |
| Gt | αt | Activation | Rhodopsin |
| | (transducin) | phosphodiesterase 6 | |
| | | (vision) | |
| Ggust | αgust | Activation | Taste receptors |
| | (gustducin) | phosphodiesterase 6 | |
| | | (vision) | |
| Gz | αz | Inhibition adenylate | unknown |
| | | cyclase (cAMP ↓) | |
| Gs family | | | |
| Gs | αs | Activation adenylate | 5-HT receptors types 5-HT4 and 5-HT7 |
| | | cyclase (cAMP ↑) | ACTH receptor |
| | | | Adenosine receptor types A2a and A2b |
| | | | Arginine vasopressin receptor 2 |
| | | | β-adrenergic receptors types β₁, β₂ and β₃ |
| | | | Calcitonin receptor |
| | | | Calcitonin gene-related peptide receptor |
| | | | Corticotropin-releasing hormone receptor |
| | | | Dopamine receptors D1-like family (D1 and |
| | | | D5) |
| | | | FSH-receptor |
| | | | Gastric inhibitory polypeptide receptor |
| | | | Glucagon receptor |
| | | | Histamine H2 receptor |
| | | | Luteinizing hormone/choriogonadotropin |
| | | | receptor |
| | | | Melanocortin receptor |
| | | | Parathyroid hormone receptor 1 |
| | | | Prostaglandin receptor types D2 and 12 |
| | | | Secretin receptor |
| | | | Thyrotropin receptor |
| Golf | αolf | Activation adenylate | Olfactory receptors |
| | | cyclase (cAMP ↑) | |
| G_{q/11} family | | | |
| G_{q} | α_{q} | Activation of | 5-HT2 serotonergic receptors |
| G₁₁ | α₁₁ | phospholipase C (IP₃ ↑) | Alpha-1 adrenergic receptor |
| | | | Vasopressin type 1 receptor |
| | | | Angiotensin II receptor type 1 |
| | | | Calcitonin receptor |
| | | | Histamine H1 receptor |
| | | | Metabotropic glutamate receptor, Group I |
| | | | M1, M3, and M5 muscarinic receptors |
| G12/13 family | | | |
| G12 | α12 | | |
| G13 | α13 | Na⁺/H⁺ exchange ↑ | |
| | βγ subunit | Effectors/Signalling | |
| | | pathways | |
| | βγ | Opening K⁺ channels | |
| | | (K⁺ ↑) | |
| | βγ | Adenylate cyclase | |
| | | (cAMP) ↑ or ↓ | |
| | βγ | Phospholipase C (IP₃) | |

Typically, in nature, G proteins are in a nucleotide-bound form. More specifically, G proteins (or at least the α subunit) are bound to either GTP or GDP depending on the activation status of a particular GPCR. Agonist binding to a GPCR promotes interactions with the GDP-bound Gαβγ heterotrimer leading to the exchange of GDP for GTP on Ga, and the functional dissociation of the G protein into Gα-GTP and Gβγ subunits. The separate Gα-GTP and Gβγ subunits can modulate, either independently or in parallel, downstream cellular effectors (channels, kinases or other enzymes, see Table A). The intrinsic GTPase activity of Gγ leads to hydrolysis of GTP to GDP and the re-association of Gα-GDP and Gβγ subunits, and the termination of signalling. Thus, G proteins serve as regulated molecular switches capable of eliciting bifurcating signals through α and βγ subunit effects. The switch is turned on by the receptor and it turns itself off within a few seconds, a time sufficient for considerable amplification of signal transduction. Methods for assessing GPCR signal transduction have been described in the art (e.g. Ratnayake et al. 2017 Methods Cellular Biology, 1:25).

The term “G_{q/11} protein” is used herein to denote G_{q} protein and G₁₁ protein, which are homologues that are 90% identical. The α₅ helices of G_{q} protein and G₁₁ protein are identical.

The term “mini-G protein” generally refers to an engineered GTPase domain of a Ga subunit.

As used herein, “mini-G_{q} protein” refers to a chimeric mini-G_{s/q} protein wherein residues of Gα_{q} that are involved in G_{q}-receptor binding and activation, in particular residues within the C-terminal region of Gα_{q} or the α₅ helix, replace the corresponding residues of mini-Gₛ protein. In particular, a mini-G_{q} protein as used herein may refer to the mini-G_{s/q}70 protein as described in Nehme et al. (2017. PLoS One 12:e0175642) which contains 7 point mutations in the α₅ helix of mini-Gₛ protein: R380K, Q384L, R385Q, H387N, Q390E, E392N and L394N.

In embodiments, the G protein peptidomimetics disclosed herein are G_{q/11} protein peptidomimetics, which are capable of biologically mimicking a G_{q/11} protein.

The G protein peptidomimetics, in particular the G_{q/11} protein peptidomimetics, described herein arise from modifications of the α₅ helix of Gα_{q/11} protein or mini-G_{q} protein, in particular from modifications of peptides comprising or consisting of the amino acid sequence set forth in SEQ ID NO: 13: FAAVKDTILQLNLKEYNLV or SEQ ID NO: 14: FNDCKDIILQMNLREYNLV, and are preferably characterized in that they are capable of stabilizing a G_{q/11} protein-coupled receptor in an active conformational state.

In particular, the G protein peptidomimetics, in particular the G_{q/11} protein peptidomimetics, may be peptides or peptide-like molecules whose amino acid sequence is derived from the amino acid sequence set forth in SEQ ID NO: 13 or SEQ ID NO: 14. Preferably, the peptidomimetics are not fragments of the α₅ helix of Gα_{q/11} protein or the α₅ helix of mini-G_{q} protein although their amino acid sequence is derived from the linear sequence of one of said as helices.

In particular, the G protein peptidomimetics, in particular the G_{q/11} protein peptidomimetics, described herein comprise or consist of a sequence of the structure (I):

(I)
(SEQ ID NO: 21)
FX₂X₃X₄KDX₇ILQX₁₁NLX₁₄X₁₅X₁₆X₁₇X₁₈X₁₉

wherein X₂ is asparagine (N) or alanine (A);
wherein X₃ is selected from the group consisting of: aspartic acid (D), alanine (A), an amino acid containing an azidated side-chain, an amino acid residue containing an alkynyl side-chain, an amino acid containing a carboxylic acid group side-chain, an amino acid containing an amine side-chain, an amino acid containing a thiol side-chain and an olefinic amino acid;
wherein X₄ is cysteine (C) or valine (V), or an amino acid without a thiol side-chain;
wherein X₇ is selected from the group consisting of: isoleucine (I), threonine (T), an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid;
wherein X₁₁ is selected from the group consisting of: methionine (M), leucine (L), an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid;
wherein X₁₄ is selected from the group consisting of: arginine (R), lysine (K), an amino acid containing an azidated side-chain or an amino acid containing an alkynyl side-chain, an amino acid containing a carboxylic acid group side-chain, an amino acid containing an amine side-chain, an amino acid containing a thiol side-chain and an olefinic amino acid;
wherein X₁₅ is glutamic acid (E), homoglutamic acid or aspartic acid (D);
wherein X₁₆ is tyrosine (Y) or an aromatic amino acid selected from the group comprising or consisting of: 4′-guanidinophenylalanine (Phe(4′guanidino)), tryptophan (W), phenylalanine (F), naphthylalanine, 1-naphthylalanine (1-NaI) and 2-naphthylalanine (2-NaI);
wherein X₁₇ is asparagine (N) or cysteine (C);
wherein X₁₈ is leucine (L), glutamic acid (E), or an alanine analogue, phenylalanine (F), tyrosine (Y), or tryptophan (W), wherein the alanine analogue is a molecule resulting from the replacement of at least one hydrogen of an alanine by at least one moiety selected from the group comprising C₆₋₁₂ cycloalkyl, C₆₋₁₂ aryl, heteroaryl, and C₆₋₁₂ cycloalkenyl; each moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl; and
wherein X₁₉ is selected from the group consisting of valine (V) or a hydrophobic amino acid, or an acidic amino acid (such as aspartic acid (D), glutamic acid (E), D-aspartic acid or D-glutamic acid).

In embodiments, the G protein peptidomimetic, in particular the G_{q/11} protein peptidomimetic, comprises or consists of a sequence of the structure (II) or (Ill):

(II)
(SEQ ID NO: 22)
FNX₃X₄KDX₇ILQX₁₁NLX₁₄X₁₅X₁₆X₁₇X₁₈X₁₉
(III)
(SEQ ID NO: 23)
FAX₃VKDX₇ILQX₁₁NLX₁₄X₁₅X₁₆X₁₇X₁₈X₁₉,

wherein X₃, X₄, X₇, X₁₅, X₁₆, X₁₇, X₁₈ and X₁₉ are as defined above.

In embodiments of the G protein peptidomimetics, in particular the G_{q/11} protein peptidomimetics, such as the G protein peptidomimetics according to any one of formula (I) to (V), (XIV), (XV) or (XVI),
when X₃ is an amino acid containing an azidated side-chain, X₇ is an amino acid containing an alkynyl side-chain,
when X₃ is an amino acid containing an alkynyl side-chain, X₇ is an amino acid containing an azidated side-chain,
when X₃ is an amino acid containing a thiol group side-chain, X₇ is an amino acid containing a thiol group side-chain,
when X₃ is an olefinic amino acid, X₇ is an olefinic amino acid,
when X₃ is an amino acid containing an amine side-chain, X₇ is an amino acid containing a carboxylic acid group side-chain, or
when X₃ is an amino acid containing a carboxylic acid group side-chain, X₇ is an amino acid containing an amine side-chain.

In embodiments of the G protein peptidomimetics, in particular the G_{q/11} protein peptidomimetics, such as the G protein peptidomimetics according to any one of formula (I) to (Ill), (VI) or (VII),
when X₇ is an amino acid containing an azidated side-chain, X₁₁ is an amino acid containing an alkynyl side-chain,
when X₇ is an amino acid containing an alkynyl side-chain, X₁₁ is an amino acid containing an azidated side-chain,
when X₇ is an amino acid containing a thiol group side-chain, X₁₁ is an amino acid containing a thiol group side-chain,
when X₇ is an olefinic amino acid, X₁₁ is an olefinic amino acid, when X₇ is an amino acid containing an amine side-chain, X₁₁ is an amino acid containing a carboxylic acid group side-chain, or
when X₇ is an amino acid containing a carboxylic acid group side-chain, X₁₁ is an amino acid containing an amine side-chain.

In embodiments of the G protein peptidomimetics, in particular the G_{q/11} protein peptidomimetics, such as the G protein peptidomimetics according to any one of formula (I) to (Ill), (Vill) or (IX),
when X₁₁ is an amino acid containing an azidated side-chain, X₁₄ is an amino acid containing an alkynyl side-chain,
when X₁₁ is an amino acid containing an alkynyl side-chain, X₁₄ is an amino acid containing an azidated side-chain,
when X₁₁ is an amino acid containing a thiol group side-chain, X₁₄ is an amino acid containing a thiol group side-chain,
when X₁₁ is an olefinic amino acid, X₁₄ is an olefinic amino acid,
when X₁₁ is an amino acid containing an amine side-chain, X₁₄ is an amino acid containing a carboxylic acid group side-chain, or
when X₁₁ is an amino acid containing a carboxylic acid group side-chain, X₁₄ is an amino acid containing an amine side-chain.

In embodiments of the G protein peptidomimetics, in particular the G_{q/11} protein peptidomimetics, such as the G protein peptidomimetics according to any one of formula (I) to (Ill), (X) or (XI),
when X₇ is an amino acid containing an azidated side-chain, X₁₄ is an amino acid containing an alkynyl side-chain,
when X₇ is an amino acid containing an alkynyl side-chain, X₁₄ is an amino acid containing an azidated side-chain,
when X₇ is an amino acid containing a thiol group side-chain, X₁₄ is an amino acid containing a thiol group side-chain,
when X₇ is an olefinic amino acid, X₁₄ is an olefinic amino acid,
when X₇ is an amino acid containing an amine side-chain, X₁₄ is an amino acid containing a carboxylic acid group side-chain, or
when X₇ is an amino acid containing a carboxylic acid group side-chain, X₁₄ is an amino acid containing an amine side-chain.

In certain embodiments, the G protein peptidomimetics, in particular the G_{q/11} protein peptidomimetics, have a peptide backbone length of 35 amino acids or less, such as 34, 33, 32, or 31 amino acids or less, preferably 30 amino acids or less, such as 29, 28, 27, 26 or 25 amino acids or less.

In the following paragraphs, different suitable, more specific, modifications that may be comprised in the peptidomimetics are described. It is evident that these different modifications may be combined into a peptidomimetic depending on the needs of individual examples and their objectives. In certain embodiments, the combination of multiple modifications is causative for a synergistic effect on the final peptidomimetic when compared to peptidomimetics comprising less modifications. However, by no means a generalization may be made that addition of modifications de facto lead to an improved peptidomimetic, and each combination should be assessed on its own merits.

G Protein Peptidomimetics with C-Terminal Modifications

In particular embodiments of the G protein peptidomimetics, in particular the G_{q/11} protein peptidomimetics, X₁₈ is an alanine analogue, phenylalanine (F), tyrosine (Y), or tryptophan (W), wherein the alanine analogue is a molecule resulting from the replacement of at least one hydrogen of an alanine by at least one moiety selected from the group comprising C₆₋₁₂ cycloalkyl, C₆₋₁₂ aryl, heteroaryl, and C₆₋₁₂ cycloalkenyl; each moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl.

The term “alanine” (code Ala or A) refers to an amino acid containing an amino group and a carboxylic acid group, both attached to the central carbon atom which also carries a methyl group side-chain.

As used herein, the term “alanine analogue” refers to a molecule resulting from the replacement of any hydrogen of alanine by at least one moiety selected from the group comprising C₁₋₆ alkyl, C₆₋₁₂ cycloalkyl, C₆₋₁₂ aryl, heteroaryl, C₆₋₁₂ cycloalkenyl; each moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, halo C₁₋₆ alkyl; or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or aryl may be optionally substituted by one or more C₁₋₆ alkyl. Preferably, the alanine analogue refers to a molecule resulting from the replacement of at least one hydrogen of the methyl group side-chain of alanine by at least one moiety selected from the group comprising C₁₋₆ alkyl, C₆₋₁₂ cycloalkyl, C₆₋₁₂ aryl, heteroaryl, and C₆₋₁₂ cycloalkenyl; each moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, halo C₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or aryl may be optionally substituted by one or more C₁₋₆ alkyl. Preferably, an alanine analogue as referred to herein comprises at least one cyclohexyl group, one phenyl group or one indole group, preferably at least one cyclohexyl group. Preferably, said cyclohexyl, phenyl or indole group is a substituent of a hydrogen of the methyl group of alanine. Optionally, said cyclohexyl, phenyl or indole group may be substituted at any position with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, halo C₁₋₆ alkyl; or 2 substituents together with the atom to which they are attached may form a C₃₋₁₂ cycloalkyl, C₅₋₁₂ cycloalkenyl, heterocycloalkyl, or C₅₋₁₂ aryl; each of said formed C₃₋₁₂ cycloalkyl, C₅₋₁₂ cycloalkenyl, heterocycloalkyl or aryl may be optionally substituted by one or more C₁₋₆ alkyl. The alanine analogues referred to herein may in addition comprise a replacement of another hydrogen of the methyl group and/or a hydrogen of the amino group of the main chain or the hydrogen atom on the alpha carbon atom.

In some embodiments, the alanine analogue, and/or X₁₈, can be a moiety of formula (Ia):

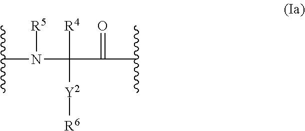

wherein
R⁴ is hydrogen or C₁₋₆ alkyl;
R⁶ is hydrogen or C₁₋₆ alkyl;
R⁶ is a moiety selected from the group comprising C₆₋₁₂ cycloalkyl, C₆₋₁₂ aryl, heteroaryl, and C₆₋₁₂ cycloalkenyl; each moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl;
Y² is —C(R⁷)R⁸— or —C(═O)—;
R⁷ is selected from the group comprising hydrogen, OH, SH, C₁₋₆ alkyl, C₃₋₁₂ cycloalkyl, C₁₋₆ alkoxy, amino, and halo;
R⁸ is hydrogen or C₁₋₆ alkyl;
or R⁷ and at least one substituent of R⁶ together with the carbon atom to which they are attached form a C₃₋₁₂ cycloalkyl, wherein said C₃₋₁₂ cycloalkyl can be optionally substituted with one or more substituents independently selected from the group comprising C₁₋₆ alkyl, OH, halo, C₃₋₁₂ cycloalkyl, C₂₋₆ alkenyl, C₁₋₆ alkoxy, oxo, ═CH₂, amino, mono- or di-C₁₋₆ alkylamino, amino C₁₋₆ alkyl, and haloC₁₋₆ alkyl, or two substituents together with the atom to which they are attached may form a C₃₋₁₂ cycloalkyl, a C₅₋₁₂ cycloalkenyl, a heterocycloalkyl or an C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl.

In some embodiments, R⁶ can be a cyclic moiety selected from the group comprising:

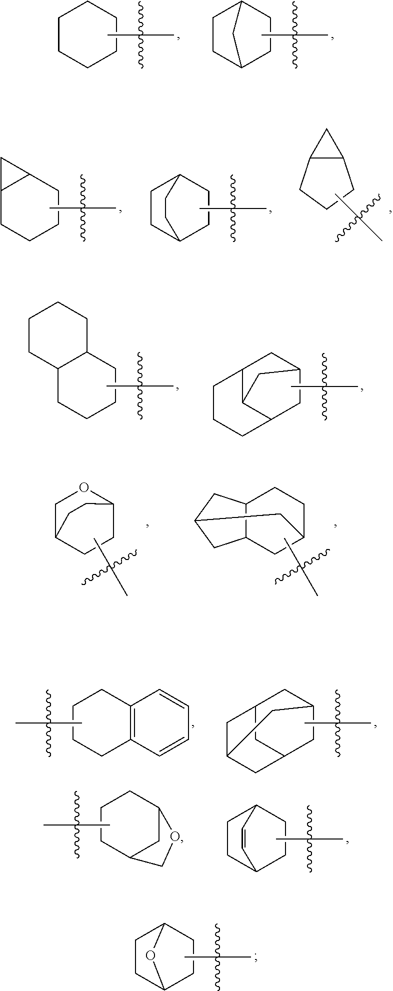

each of said cyclic moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl.

Preferably, in some embodiments, R⁶ can be a cyclic moiety selected from the group comprising

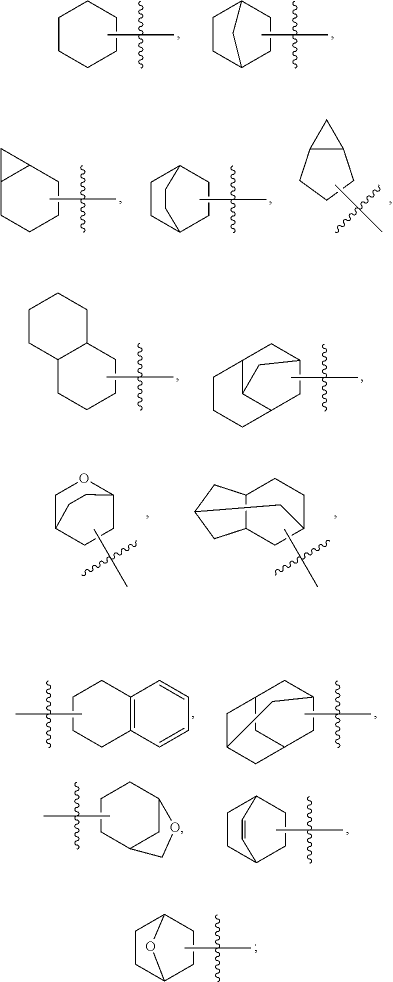

each of said cyclic moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl.

Preferably, in some embodiments, R⁶ can be a cyclic moiety selected from the group comprising

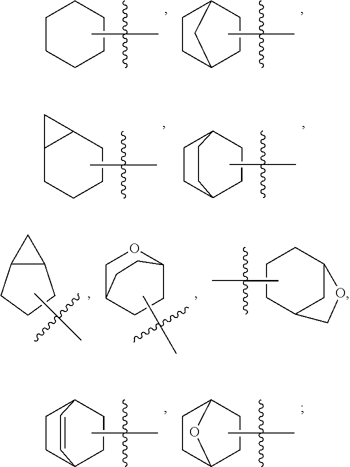

each of said cyclic moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl.

Preferably, in some embodiments, R⁶ can be a cyclic moiety selected from the group comprising

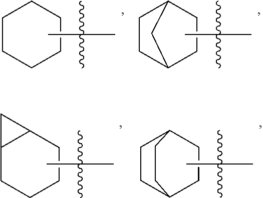

each of said cyclic moiety being optionally substituted with one or more substituents each independently selected from OH, halo, C₁₋₆ alkyl, C₁₋₆ alkoxy, oxo, ═CH₂, haloC₁₋₆ alkyl.

Non-limiting examples of cyclohexylalanine analogues to be used in the preparation of the peptidomimetic are shown in Table B:

TABLE B
Non-limiting examples of suitable cyclohexylalanine analogues.

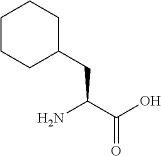 [SMILES: N[C@@H](CC1CCCCC1)C(=O)O]

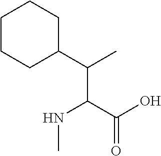

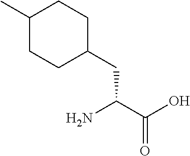

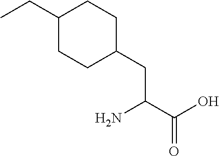

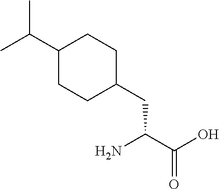

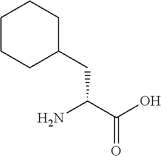 [SMILES: N[C@H](CC1CCCCC1)C(=O)O]

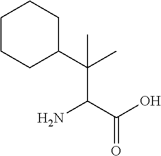

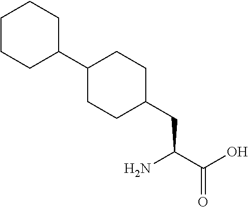 [SMILES: N[C@@H](CC1CCC(C2CCCCC2)CC1)C(=O)O]

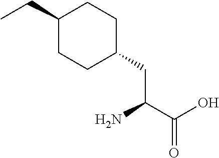

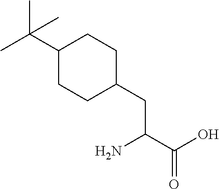 [SMILES: CC(C)(C)C1CCC(CC(N)C(=O)O)CC1]

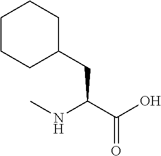

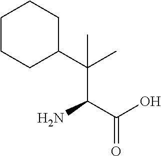

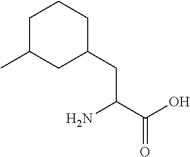

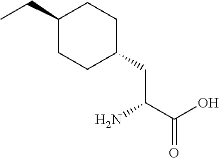

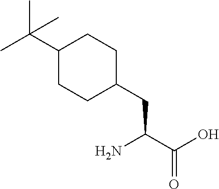 [SMILES: CC(C)(C)C1CCC(C[C@H](N)C(=O)O)CC1]

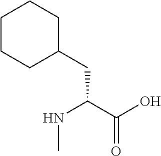

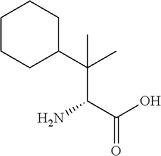

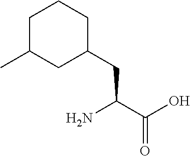

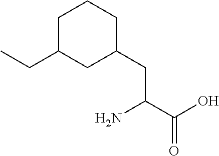

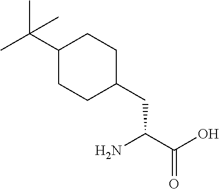 [SMILES: CC(C)(C)C1CCC(C[C@@H](N)C(=O)O)CC1]

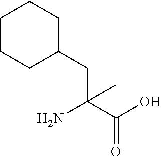

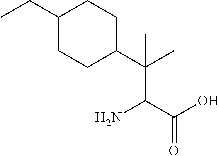 [SMILES: CCC1CCC(C(C)(C)C(N)C(=O)O)CC1]

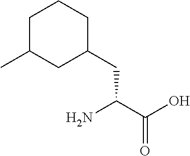

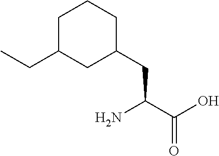

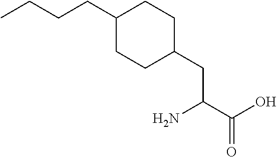 [SMILES: CCCCC1CCC(CC(N)C(=O)O)CC1]

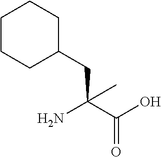

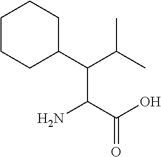

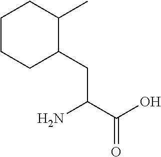

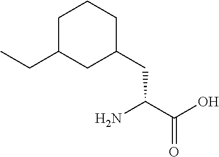

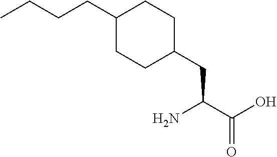 [SMILES: CCCCC1CCC(C[C@H](N)C(=O)O)CC1]

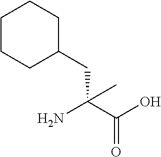

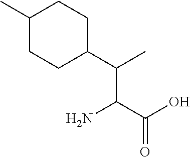

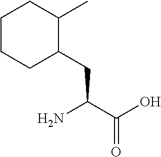

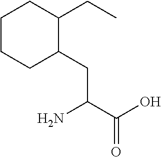

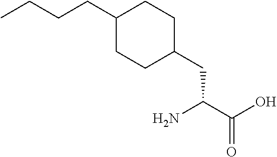 [SMILES: CCCCC1CCC(C[C@@H](N)C(=O)O)CC1]

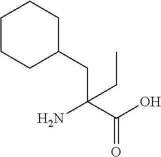

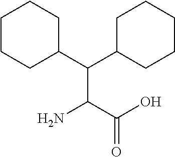 [SMILES: NC(C(=O)O)C(C1CCCCC1)C1CCCCC1]

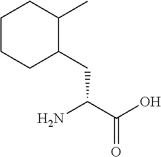

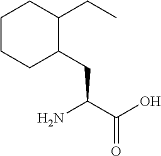

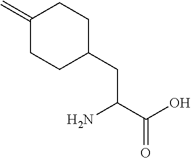

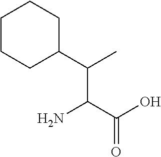

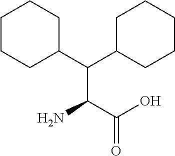 [SMILES: N[C@H](C(=O)O)C(C1CCCCC1)C1CCCCC1]

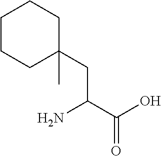

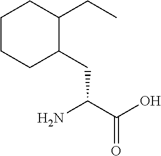

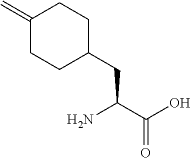

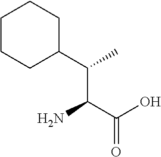

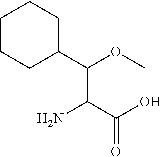

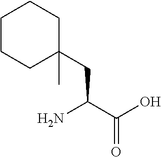

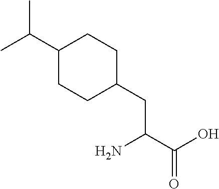

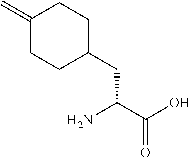

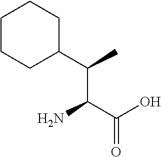

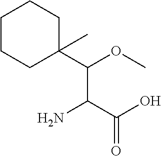

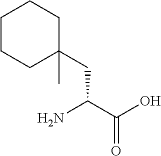

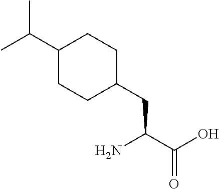

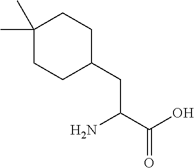

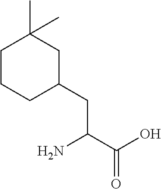

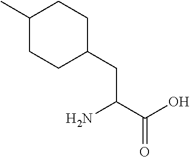

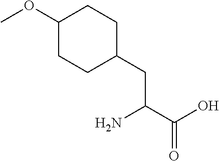

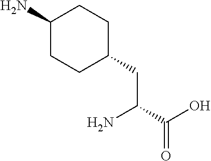 [SMILES: N[C@H](C[C@H]1CC[C@H](N)CC1)C(=O)O]

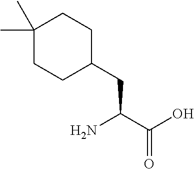

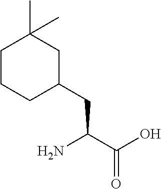

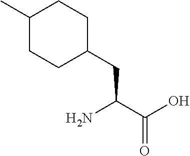

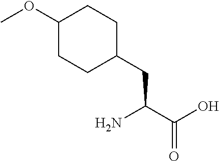

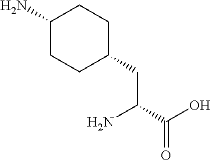 [SMILES: N[C@H](C[C@H]1CC[C@@H](N)CC1)C(=O)O]

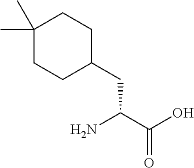

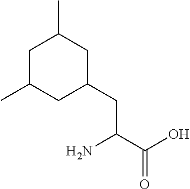

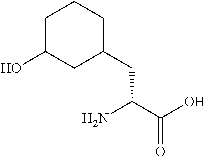 [SMILES: N[C@H](CC1CCCC(O)C1)C(=O)O]

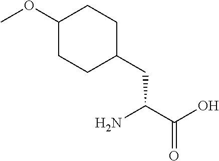

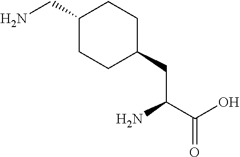 [SMILES: NC[C@H]1CC[C@H](C[C@H](N)C(=O)O)CC1]

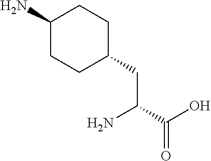 [SMILES: N[C@H](C[C@H]1CC[C@H](N)CC1)C(=O)O]

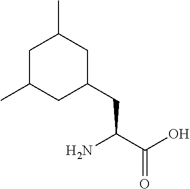

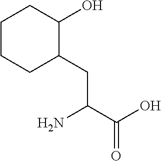 [SMILES: NC(CC1CCCCC1O)C(=O)O]

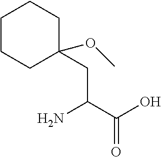

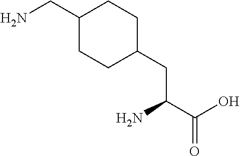 [SMILES: NCC1CCC(C[C@H](N)C(=O)O)CC1]

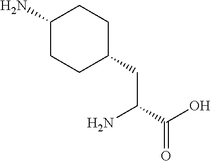 [SMILES: N[C@H](C[C@H]1CC[C@@H](N)CC1)C(=O)O]

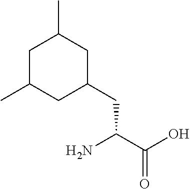

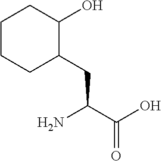 [SMILES: N[C@@H](CC1CCCCC1O)C(=O)O]

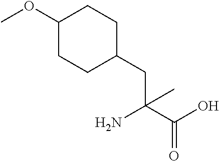

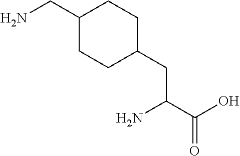 [SMILES: NCC1CCC(CC(N)C(=O)O)CC1]

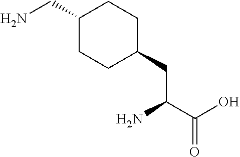 [SMILES: NC[C@H]1CC[C@H](C[C@H](N)C(=O)O)CC1]

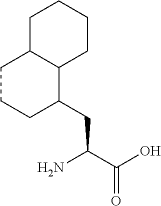

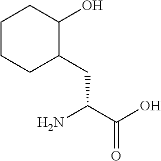 [SMILES: N[C@H](CC1CCCCC1O)C(=O)O]

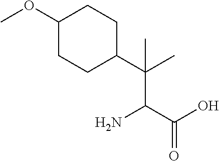 [SMILES: COC1CCC(C(C)(C)C(N)C(=O)O)CC1]

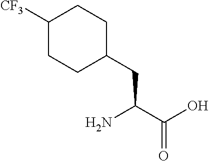 [SMILES: N[C@@H](CC1CCC(C(F)(F)F)CC1)C(=O)O]

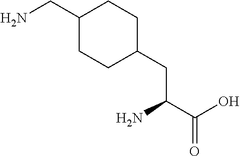 [SMILES: NCC1CCC(C[C@H](N)C(=O)O)CC1]

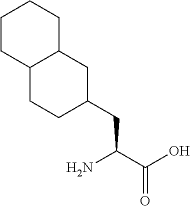 [SMILES: N[C@@H](CC1CCC2CCCCC2C1)C(=O)O]

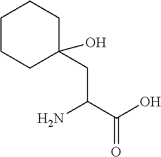 [SMILES: NC(CC1(O)CCCCC1)C(=O)O]

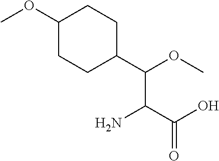

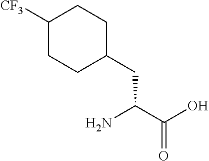 [SMILES: N[C@H](CC1CCC(C(F)(F)F)CC1)C(=O)O]

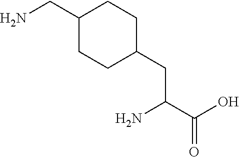 [SMILES: NCC1CCC(CC(N)C(=O)O)CC1]

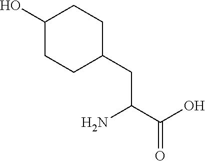 [SMILES: NC(CC1CCC(O)CC1)C(=O)O]

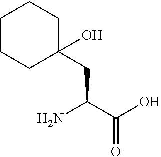 [SMILES: N[C@@H](CC1(O)CCCCC1)C(=O)O]

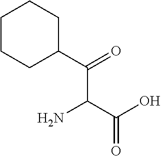 [SMILES: NC(C(=O)O)C(=O)C1CCCCC1]

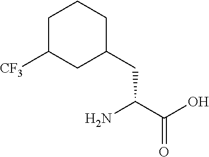 [SMILES: N[C@H](CC1CCCC(C(F)(F)F)C1)C(=O)O]

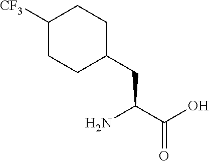 [SMILES: N[C@@H](CC1CCC(C(F)(F)F)CC1)C(=O)O]

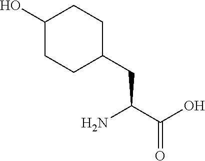 [SMILES: N[C@@H](CC1CCC(O)CC1)C(=O)O]

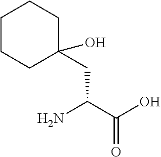 [SMILES: N[C@H](CC1(O)CCCCC1)C(=O)O]

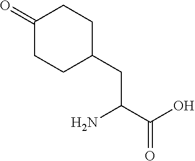 [SMILES: NC(CC1CCC(=O)CC1)C(=O)O]

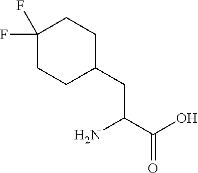 [SMILES: NC(CC1CCC(F)(F)CC1)C(=O)O]

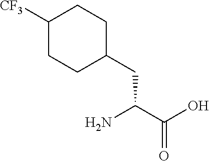 [SMILES: N[C@H](CC1CCC(C(F)(F)F)CC1)C(=O)O]

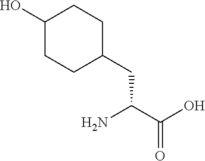 [SMILES: N[C@H](CC1CCC(O)CC1)C(=O)O]

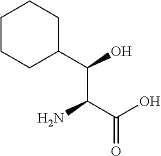 [SMILES: N[C@H](C(=O)O)[C@H](O)C1CCCCC1]

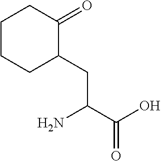 [SMILES: NC(CC1CCCCC1=O)C(=O)O]

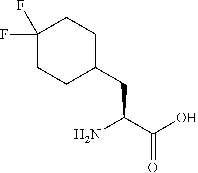 [SMILES: N[C@@H](CC1CCC(F)(F)CC1)C(=O)O]

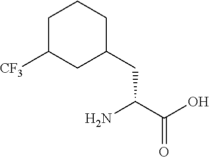 [SMILES: N[C@H](CC1CCCC(C(F)(F)F)C1)C(=O)O]

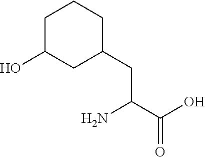 [SMILES: NC(CC1CCCC(O)C1)C(=O)O]

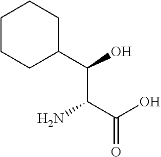 [SMILES: N[C@@H](C(=O)O)[C@H](O)C1CCCCC1]

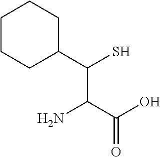 [SMILES: NC(C(=O)O)C(S)C1CCCCC1]

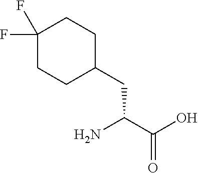 [SMILES: N[C@H](CC1CCC(F)(F)CC1)C(=O)O]

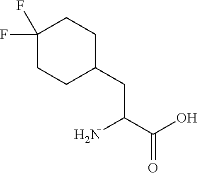 [SMILES: NC(CC1CCC(F)(F)CC1)C(=O)O]

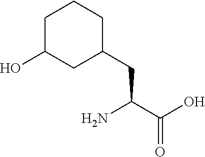 [SMILES: N[C@@H](CC1CCCC(O)C1)C(=O)O]

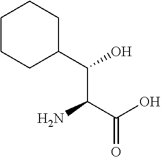 [SMILES: N[C@H](C(=O)O)[C@@H](O)C1CCCCC1]

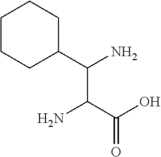 [SMILES: NC(C(=O)O)C(N)C1CCCCC1]

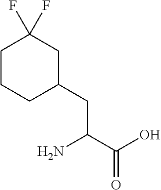 [SMILES: NC(CC1CCCC(F)(F)C1)C(=O)O]

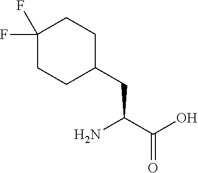 [SMILES: N[C@@H](CC1CCC(F)(F)CC1)C(=O)O]

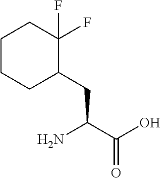 [SMILES: N[C@@H](CC1CCCCC1(F)F)C(=O)O]

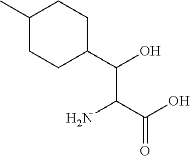

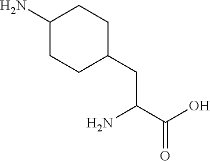 [SMILES: NC1CCC(CC(N)C(=O)O)CC1]

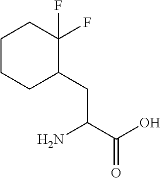 [SMILES: NC(CC1CCCCC1(F)F)C(=O)O]

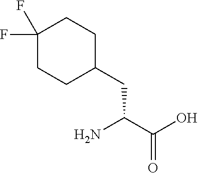 [SMILES: N[C@H](CC1CCC(F)(F)CC1)C(=O)O]

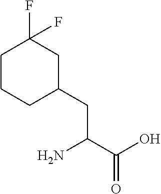 [SMILES: NC(CC1CCCC(F)(F)C1)C(=O)O]

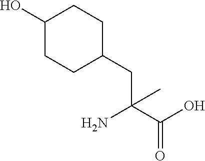

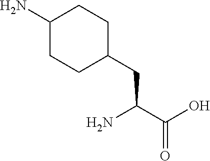 [SMILES: NC1CCC(C[C@H](N)C(=O)O)CC1]

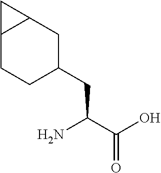 [SMILES: N[C@@H](CC1CCC2CC2C1)C(=O)O]

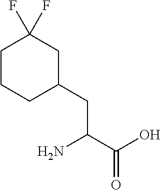 [SMILES: NC(CC1CCCC(F)(F)C1)C(=O)O]

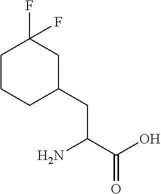 [SMILES: NC(CC1CCCC(F)(F)C1)C(=O)O]

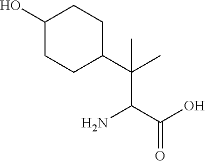

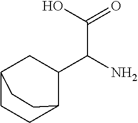 [SMILES: NC(C(=O)O)C1CC2CCC1CC2]

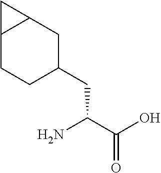 [SMILES: N[C@H](CC1CCC2CC2C1)C(=O)O]

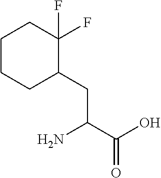 [SMILES: NC(CC1CCCCC1(F)F)C(=O)O]

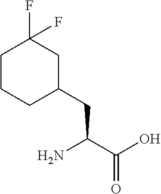 [SMILES: N[C@@H](CC1CCCC(F)(F)C1)C(=O)O]

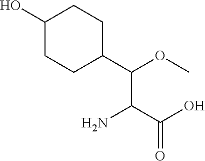

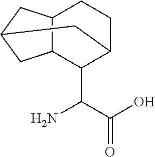 [SMILES: NC(C(=O)O)C1C2CCC3CC(C2)CC31]

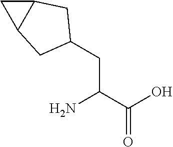 [SMILES: NC(CC1CC2CC2C1)C(=O)O]

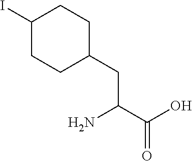 [SMILES: NC(CC1CCC(I)CC1)C(=O)O]

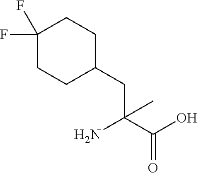

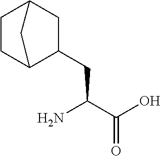 [SMILES: N[C@@H](CC1CC2CCC1C2)C(=O)O]

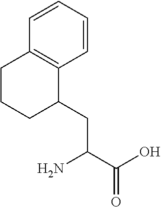 [SMILES: NC(CC1CCCc2ccccc21)C(=O)O]

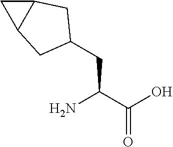 [SMILES: N[C@@H](CC1CC2CC2C1)C(=O)O]

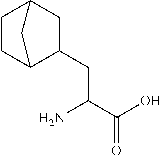 [SMILES: NC(CC1CC2CCC1C2)C(=O)O]

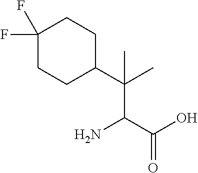 [SMILES: CC(C)(C1CCC(F)(F)CC1)C(N)C(=O)O]

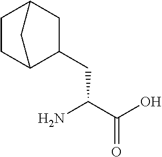 [SMILES: N[C@H](CC1CC2CCC1C2)C(=O)O]

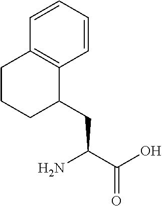 [SMILES: N[C@@H](CC1CCCc2ccccc21)C(=O)O]

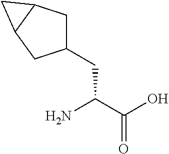 [SMILES: N[C@H](CC1CC2CC2C1)C(=O)O]

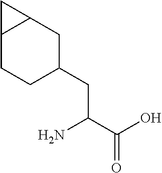 [SMILES: NC(CC1CCC2CC2C1)C(=O)O]

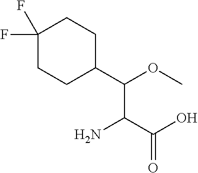 [SMILES: COC(C1CCC(F)(F)CC1)C(N)C(=O)O]

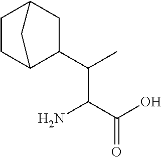

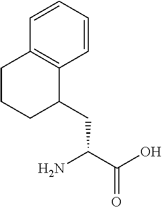 [SMILES: N[C@H](CC1CCCc2ccccc21)C(=O)O]

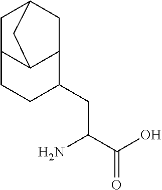 [SMILES: NC(CC1CCC2CC3CC2C1C3)C(=O)O]

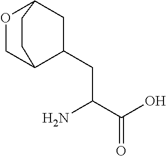 [SMILES: NC(CC1CC2CCC1CO2)C(=O)O]

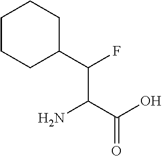 [SMILES: NC(C(=O)O)C(F)C1CCCCC1]

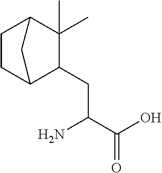

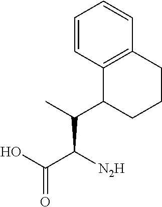

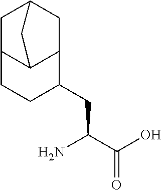 [SMILES: N[C@@H](CC1CCC2CC3CC2C1C3)C(=O)O]

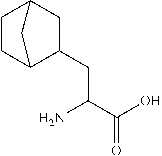 [SMILES: NC(CC1CC2CCC1C2)C(=O)O]

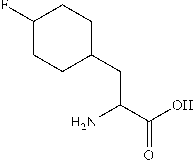 [SMILES: NC(CC1CCC(F)CC1)C(=O)O]

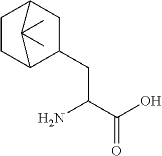

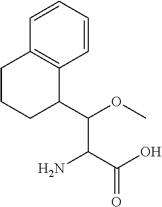 [SMILES: COC(C(N)C(=O)O)C1CCCc2ccccc21]

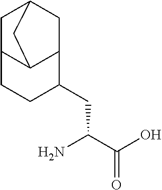 [SMILES: N[C@H](CC1CCC2CC3CC2C1C3)C(=O)O]

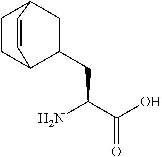 [SMILES: N[C@@H](CC1CC2C=CC1CC2)C(=O)O]

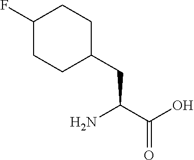 [SMILES: N[C@@H](CC1CCC(F)CC1)C(=O)O]

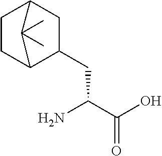

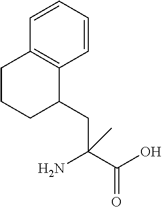 [SMILES: CC(N)(CC1CCCc2ccccc21)C(=O)O]

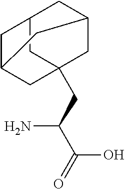 [SMILES: N[C@@H](CC12CC3CC(CC(C3)C1)C2)C(=O)O]

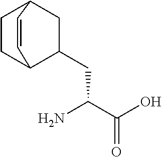 [SMILES: N[C@H](CC1CC2C=CC1CC2)C(=O)O]

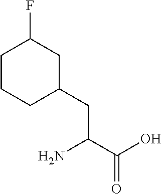 [SMILES: NC(CC1CCCC(F)C1)C(=O)O]

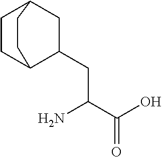 [SMILES: NC(CC1CC2CCC1CC2)C(=O)O]

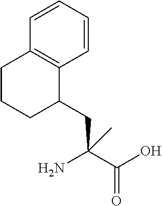 [SMILES: C[C@](N)(CC1CCCc2ccccc21)C(=O)O]

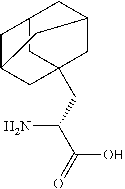 [SMILES: N[C@H](CC12CC3CC(CC(C3)C1)C2)C(=O)O]

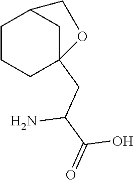 [SMILES: NC(CC12CCCC(CO1)C2)C(=O)O]

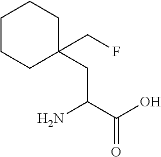 [SMILES: NC(CC1(CF)CCCCC1)C(=O)O]

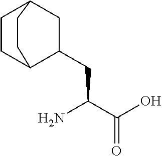 [SMILES: N[C@@H](CC1CC2CCC1CC2)C(=O)O]

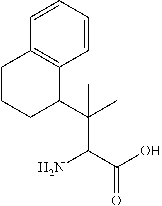 [SMILES: CC(C)(C(N)C(=O)O)C1CCCc2ccccc21]

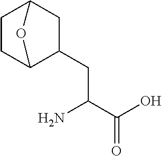 [SMILES: NC(CC1CC2CCC1O2)C(=O)O]

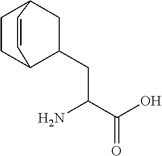 [SMILES: NC(CC1CC2C=CC1CC2)C(=O)O]

In some embodiments, the alanine analogue, and/or X₁₈ is a moiety of formula (Ib):

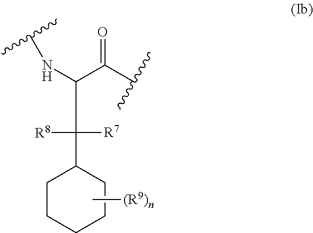

wherein R⁷ is selected from the group comprising hydrogen, OH, SH, C₁₋₆ alkyl, C₃₋₁₂ cycloalkyl, C₁₋₆ alkoxy, amino, and halo;
R⁸ is hydrogen or C₁₋₆ alkyl;
n is an integer selected from 0, 1, 2, 3, 4, or 5; preferably 1, 2, 3 or 4, preferably 1, 2 or 3;
R⁹ is selected from the group comprising OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or two R⁹ together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl;
or R⁷ and at least one R⁹ together with the carbon atom to which they are attached form a C₃₋₁₂ cycloalkyl, wherein said C₃₋₁₂ cycloalkyl can be optionally substituted with one or more substituents independently selected from the group comprising C₁₋₆ alkyl, OH, halo, C₃₋₁₂ cycloalkyl, C₂₋₆ alkenyl, C₁₋₆ alkoxy, oxo, ═CH₂, amino, mono- or di-C₁₋₆ alkylamino, amino C₁₋₆ alkyl, and haloC₁₋₆ alkyl, or two substituents together with the atom to which they are attached may form a C₃₋₁₂ cycloalkyl, a C₅₋₁₂ cycloalkenyl, a heterocycloalkyl or an C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl.

In some embodiments, the alanine analogue, and/or X₁₈ is a moiety of formula (Ic):

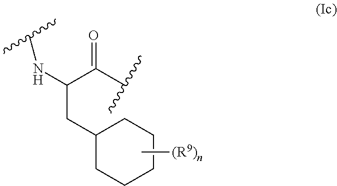

wherein n is an integer selected from 0, 1, 2, 3, 4, or 5; preferably 1, 2, 3 or 4, preferably 1, 2 or 3;
wherein R⁹ is selected from the group comprising OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or two R⁹ together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl.

In embodiments, X₁₈ is selected from the group consisting of cyclohexylalanine, phenylalanine, tyrosine and tryptophan, each of said cyclohexylalanine, phenylalanine, tyrosine and tryptophan being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl. Preferably, X₁₈ is cyclohexylalanine said cyclohexylalanine being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl.

In embodiments, X₁₈ is selected from the group consisting of cyclohexylalanine, phenylalanine, tyrosine and tryptophan.

In embodiments, X₁₈ is cyclohexylalanine.

In embodiments, X₁₈ is selected from the group consisting of phenylalanine, tyrosine and tryptophan. Without wishing to be bound by any theory, the aromatic residue (phenylalanine, tyrosine and/or tryptophan) may target a receptor arginine in close proximity (e.g. R¹²³ of M1R as defined by SEQ ID NO: 15; R¹²⁵ of H1R as defined by SEQ ID NO: 16 or R¹⁷³ of 5-HT_{2A}R as defined by SEQ ID NO: 17) to induce a cation-T interaction.

In other embodiments of the G protein peptidomimetics, in particular the G_{q/11} protein peptidomimetics, X₁₈ is glutamic acid (E). Without wishing to be bound by any theory, the glutamic acid may create a hydrogen bond with a receptor arginine in close proximity (e.g. R¹²³ of M1R as defined by SEQ ID NO: 15; R¹²⁵ of H1R as defined by SEQ ID NO: 16; and/or R¹⁷³ of 5-HT_{2A}R as defined by SEQ ID NO: 17).

In embodiments of the G protein peptidomimetics, in particular the G_{q/11} protein peptidomimetics, X₁₉ is an acidic amino acid, preferably X₁₉ is selected from the group consisting of aspartic acid (D), glutamic acid (E), D-aspartic acid or D-glutamic acid, more preferably X₁₉ is D-aspartic acid or D-glutamic acid. The acidic amino acids may target receptor basic amino acid residues (e.g. T²¹⁵, R²¹⁸, K³⁶¹ and/or K³⁶² of MIR as defined by SEQ ID NO: 15; L⁴⁰⁵(BB), R⁴⁰⁹ and/or K⁴¹² of H1R as defined by SEQ ID NO: 16; and/or N³¹⁷, K³²⁰, N³⁸⁴ and/or K³⁸⁵ of 5-HT_{2A}R as defined by SEQ ID NO: 17). The D-stereoisomers may advantageously improve the orientation for additional interactions.

In embodiments of the G protein peptidomimetics, in particular the G_{q/11} protein peptidomimetics, X₁₇ is cysteine (C). Without wishing to be bound by any theory, the cysteine may allow a covalent disulfide bridge between the G protein peptidomimetic and the GPCR (e.g. C⁴²¹ of M1R as defined by SEQ ID NO: 15; and/or C⁴⁷¹ of H1R as defined by SEQ ID NO: 16). To avoid intramolecular cyclization, the peptidomimetic preferably does not contain a cysteine residue at its N-terminus. In embodiments, X₁₇ is cysteine (C) and X₄ is an amino acid without a thiol side-chain.

In embodiments of the G protein peptidomimetics, in particular the G_{q/11} protein peptidomimetics, X₁₆ is an aromatic amino acid selected from the group comprising or consisting of: 4′-guanidinophenylalanine (Phe(4′guanidino)), tryptophan (W), phenylalanine (F), naphthylalanine, 1-naphthylalanine (1-NaI) and 2-naphthylalanine (2-NaI), preferably X₁₆ is 4′-guanidinophenylalanine (Phe(4′-guanidino)). Without wishing to be bound by any theory, the Phe(4′-guanidino) may keep the cation-n interaction with the proximal arginine and increase the number of hydrogen bonds (e.g. (e.g. N⁶⁰, D¹²², S¹²⁶ and/or R¹²³ of MIR as defined by SEQ ID NO: 15; D¹²⁴, S¹²⁸, N⁴⁷² and/or R¹²⁵ of H1R as defined by SEQ ID NO: 16; and/or T¹⁰⁹, D¹⁷² and/or R¹⁷³ of 5-HT_{2A}R as defined by SEQ ID NO: 17).

In embodiments of the G protein peptidomimetics, in particular the G_{q/11} protein peptidomimetics, X₁₅ is homoglutamic acid or aspartic acid (D), preferably homoglutamic acid. The homoglutamic acid and the aspartic acid, in particular the homoglutamic acid, may decrease the distance and strengthen the interactions with the residues in the binding pocket of the GPCR (e.g. N⁶⁰, N⁶¹ and/or N⁴²² of MIR as defined by SEQ ID NO: 15; T⁶⁰, R¹³⁹ and/or N⁴⁷² of H1R as defined by SEQ ID NO: 16; and/or N¹⁰⁷, N¹⁸⁷ and/or R¹⁸⁹ of 5-HT_{2A}R as defined by SEQ ID NO: 17).

### Covalently Tethered G Protein Peptidomimetics

In embodiments, the G protein peptidomimetics, in particular the G_{q/11} protein peptidomimetics, are (macro)cyclized or covalently tethered (‘stapled’), i.e. an intramolecular covalent bond, tether or linkage is formed between two non-adjacent (amino acid) residues of the peptide or peptidomimetic. These cyclized peptides have also been coined “macrocycles” in the art. Both peptidomimetics comprising a staple and peptidomimetics comprising suitable (amino acid) residues arranged to allow (macro)cyclization are envisaged herein. Any of the sequences disclosed herein can refer to a peptide or a peptidomimetic wherein (side-chains of) residues have been reacted to form a covalent tether as described herein, i.e. a stapled peptide or peptidomimetic.

(Macro)cyclization or stapling of the peptide or peptidomimetic disclosed herein is aimed to stabilize and/or mimic peptide α-helices. The α-helical secondary structure is well defined in the art. Briefly, they comprise a right-handed spiral that is maintained by hydrogen bond interactions between the hydrogen from the backbone amino group of an amino acid of the peptide and the backbone carbonyl group of the amino acid in a further position (3 or 4 residues) of the peptide chain. Methods to measure the helicity of a peptide are known to a person skilled in the art, such as but not limited to circular dichroism, nuclear magnetic resonance (NMR) spectroscopy, and X-ray crystallography.

Stapling of the peptides may confer certain advantages over their non-stapled counterparts, or improve certain advantages observed to a lesser degree in the non-stapled counterparts. Such advantages may include but are not limited to (improved) protease resistance and/or (improved) cellular uptake.

Different combinations of functional group to achieve macrocyclization have been described in the art and include head-to-side-chain (i.e. between the N-terminus of the peptide and a functional group on a side-chain of an amino acid), head-to-tail (i.e. between the N-terminus and C-terminus), side-chain-to-tail (i.e. between the C-terminus and a functional group on a side-chain of an amino acid), and side-chain-to-side-chain (between two functional groups on the side-chain of an amino acid). In a preferred embodiment, the G protein peptidomimetics, in particular the G_{q/11} protein peptidomimetics, comprise at least one side-chain-to-side-chain cyclization that stabilizes an α-helical conformation. The cyclization may be formed between two natural occurring amino acids, between two non-naturally occurring amino acids, or between a naturally occurring and a non-naturally occurring amino acid.

Cyclization may be achieved by methods well-known to those in the art (described inter alia in detail in White and Yudin (2011. Nature Chemistry 3:509-524) and Lau et al. (2014. Chem. Soc. Rev. 44:91-102). Non-limiting examples of cyclization reactions include Ugi reaction, lactamization, ring-closing metathesis (RCM), triazole formation by copper-catalyzed azide-alkyne cycloaddition (CuAAC) (also referred to as click chemistry), Staudinger ligation, thiol-ene addition, thiazolidine formation, cross-coupling, disulfide formation, and azobenzene formation, as known to the skilled person. In view of the preferred side-to-side-chain cyclization manner of peptidomimetics as described herein, non-limiting examples of preferred cyclization reactions include cross-coupling, ring-closing metathesis, lactamization, disulfide formation, and azobenzene formation. In further preferred embodiments, the macrocyclizations are generated by ring-closing metathesis, lactamization, disulfide bridge formation, or click chemistry.

Macrocyclization by a lactamization reaction is based on the formation of an amide bond between two amino acid side-chains. Exemplary pairs of amino acids that are suitable for this reaction are aspartic acid (D) or glutamic acid (E) (providing the carboxylic group) and lysine (K), ornithine (Orn) or diaminopropionic acid (Dap) (providing the amine group). It is common in the art to discriminate lactamization reactions wherein the amino acid providing the amine functional group is positioned as the C-terminal or N-terminal amino acid in the reaction. In the latter case, such a lactam bridge is typically referred to as a “reverse” lactam bridge. Both “standard” lactam bridges and “reverse” lactam bridges are envisaged by the present disclosure, as both induce a secondary structure on the peptide showing similarity to a α-helix. In a lactamization reaction, an amide is formed by condensation between a carboxylic acid and an amine wherein a water molecule is eliminated. Lactamization reactions require a condensation agent in order to activate the carboxylic group. Numerous suitable condensation agents have been described in the art and include but are by no means limited to carbodiimides (e.g. N,N′-dicyclohexylcarbodiimide (DCC) and N,N′-diisopropylcarbodiimide (DIC), phosphonium salts (e.g. (Benzotriazol-1-yloxy)tris(dimethylamino)phosphonium hexafluorophosphate (BOP) or (Benzotriazol-1-yloxy)tripyrrolidinophosphonium hexafluorophosphate (PyBOP)), uronium salts, or thiouronium salts (HBTU, TOTT). While one of the strengths of lactamization is the possibility to rely on natural amino acids, a skilled person appreciates that this cyclization method may also be used between any combination of naturally or non-naturally occurring amino acids that are able to form an amide bond by condensation of a carboxylic acid-containing side-chain of a first amino acid and an amine-containing side-chain of a second amino acid. Additionally, functional groups involved in stapling should be protected during peptide synthesis by protective groups orthogonal to the protective groups used for the N- and or C-termini.

Macrocyclization by ring-closing metathesis is based on coupling of two terminal alkenes that form a macrocycle linked by a double bond with the loss of an ethylene molecule. Central to ring-closing metathesis is a metal catalyst. Different catalysts have been described in the art and include but are not limited to so-called first and second generation Grubbs catalysts, and Shrock catalysts. First generation Grubbs catalysts have a ruthenium core substituted with two phosphine groups, two chlorine atoms and a carbene compound and have the advance of being air-stable and therefore easy to handle. Second generation Grubbs catalysts comprise an N-heterocyclic carbene (NHC) replacing a phosphine substituent. NHC provides enhanced catalyst activity while still providing adequate air and water stability. The reaction relies on a double 2+2 cycloaddition—cycloelimination between an olefin (as envisaged herein an olefinic substituted amino acid) and the carbene-metal complex. While the thermal cycloaddition between olefinic compounds require high activation energies since they are symmetry forbidden, interaction with the metal catalysts substantially lower the activation energy, allowing the reaction to occur at room temperature. As indicated above, ring-closing metathesis requires two olefinic-substituted amino acids. A non-limiting manner to generate such amino acids is by allylation of serine by a nucleophilic substitution reaction between the hydroxyl group of serine and an allyl halide. Alternatively, insertion of a terminal olefinic hydrocarbon chain on glycine or alanine residues may be achieved by usage of a chiral nickel catalyst. Non-limiting examples of suitable olefinic amino acids include alanine derivatives “S5”, “R5” and “R8” as described herein. In the art, the terms “alkene” and “olefin” are often used interchangeably.

Macrocyclization by disulfide formation can also be envisaged. Disulfide bridges may be formed between amino acids that have a thiol-side-chain such as e.g. cysteine. Disulfide bridges are typically formed by oxidation of the sulfhydryl groups.

Copper-catalyzed azide-alkyne cycloaddition (CuAAC) is a further preferred macrocyclization reaction and the reaction as such is alternatively known in the art as “Huisgen cycloaddition” or Click-chemistry. An advantage of this approach is that the functional groups that are involved are orthogonal to any other functionality in a cellular milieu. Additionally, the copper catalysis is typically performed under mild conditions. CuAAC relies on regioselective 3+2 cycloaddition between an azide and a terminal alkyne leading to a 1,4-disubstituted 1,2,3-triazole ring having aromatic properties. In a CuAAC reaction, copper is linked to an alkyne. The subsequent elimination of the terminal proton is responsible for formation of a copper-acetylide complex. In a next step, the azido group is linked to the copper atom, eventually forming a triazoic ring which is then released. Methods to generate alkynyl-amino acids have been described in the art. A non-limiting suitable method is nucleophilic substitution on a propargyl bromide or homolog thereof by a nucleophilic amino acid. The nucleophilic amino acid may be a natural nucleophilic amino acid such as serine, cysteine, glutamate, glutamine, aspartic acid, or asparagine. Alternatively, a chiral nickel catalyst can be employed to obtain (all-hydrocarbon) alkynyl amino acids. Methods to generate azidated amino acids are ubiquitous in the art (and are inter alia summarized in Johansson and Pedersen 2012 European Journal of Organic Chemistry 4267-4281). Illustrative methods include direct insertion of the azido group on a serine residue by using Mitsunobu coupling conditions, mesylation of the serine Weinreb amide and subsequent insertion of the azido group by nucleophilic substitution on the mesylated hydroxyl group, Hoffmann rearrangement of an asparagine followed by a diazotransfer, or Ullmann coupling of p-iodophenylalanine. Non-limiting examples of a suitable amino acid containing an azidated side-chain are azidolysine (also referred to herein as “Azk”), norleucine(εN3) (Nle(εN3)) and norvaline(δN3) (Nva(δN3)); non-limiting examples of a suitable amino acid containing an alkynyl side-chain are propargylglycine (also referred to herein as “Pra”) and propargylalanine (Paa). Non-limiting examples of suitable CuAAC cyclizations are between azidolysine and propargylglycine, between norleucine(εN3) and propargylglycine (Pra), between norvaline(δN³) and propargylglycine, between norleucine(εN3) and propargylalanine, and between norvaline(δN³) and propargylalanine.

The terms “covalent tether”, “tether”, “staple”, “braces”, “bridges” may be used interchangeably herein and are to be interpreted in the current disclosure in accordance with their generally accepted meaning in the technical field, i.e. a covalent tether or bond that is not part of the linear peptide backbone and that mediates macrocycle formation.

For example, and without limitation, in the G protein peptidomimetics, in particular the G_{q/11} protein peptidomimetics, disclosed herein, a staple can be formed from the reaction/coupling of the side-chain of X₃ with the side-chain of X₇, from the reaction/coupling of the side-chain of X₇ with the side-chain of X₁₁, from the reaction/coupling of the side-chain of X₁₁ with the side-chain of X₁₄, or from the reaction/coupling of the side-chain of X₇ with the side-chain of X₁₄.

In embodiments, the G protein peptidomimetic, in particular the G_{q/11} protein peptidomimetic, comprises a covalent tether between X₃ and X₇, between X₇ and X₁₁, between X₁₁ and X₁₄, or between X₇ and X₁₄, preferably between X₃ and X₇, wherein said covalent tether is not part of the linear peptide backbone.

In further embodiments, said covalent tether is formed between an amino acid containing an azidated side-chain and an amino acid containing an alkynyl-bearing side-chain. In certain embodiments, said covalent tether is formed between an azidolysine (Azk) and a propargylglycine (Pra). In alternative further embodiments, said covalent tether is formed between an amino acid containing an amine side-chain and an amino acid containing a carboxylic acid group side-chain. In certain embodiments, the covalent tether is a lactam bridge formed between a glutamic acid and a lysine or between an aspartic acid and a lysine. In alternative further embodiments, said covalent tether is formed between two olefinic amino acids. In yet alternative further embodiments, the staple is a disulfide bridge connecting two amino acids each comprising a thiol functional group in their side-chains. In certain embodiments, a disulfide bridge is formed between two cysteine residues. The order of disclosure in the above embodiments does by no means imply that such an arrangement is fixed in the herein disclosed peptidomimetics.

In particular embodiments, a staple is formed from the reaction/coupling of the side-chain of X₃ with the side-chain of X₇, wherein X₇ is an amino acid containing an azidated side-chain and X₃ is an amino acid containing an alkynyl side-chain or wherein X₃ is an amino acid containing an azidated side-chain and X₇ is an amino acid containing an alkynyl side-chain. In yet further particular embodiments, a staple is formed from the reaction/coupling of the side-chain of X₃ with the side chain of X₇, wherein X₇ is an azidolysine (Azk) and X₃ is a propargylglycine (Pra) or wherein X₃ is an azidolysine (Azk) and X₇ is a propargylglycine (Pra). Particularly preferred embodiments are peptidomimetics wherein a staple is formed from the reaction/coupling of the side-chain of X₃ with the side chain of X₇, wherein X₇ is an azidolysine (Azk) and X₃ is a propargylglycine (Pra).

The G protein peptidomimetics, in particular the G_{q/11} protein peptidomimetics, disclosed herein can also comprise two or more staples. For example, a peptidomimetic may comprise a side-chain-to-side-chain macrocyclization in addition to a second side-chain-to-side-chain macrocyclization formed by identical, similar, or unrelated functional groups of a side-chain of an amino acid. In such an example, four amino acids of the peptidomimetic would be used to generate a double staple (i.e. two braces). Furthermore, a peptidomimetic may comprise a side-chain-to-side-chain macrocyclization combined with any other macrocyclization of any head, tail, or side-chain combination. Thus, any type of staple able to stabilize the linear peptidomimetic in a helix conformation as described herein may be used in connection with any of the macrocyclization methods known in the art.

### Linear G Protein Peptidomimetics

In other embodiments, the G protein peptidomimetics, in particular the G_{q/11} protein peptidomimetics, are linear. Linear G protein peptidomimetics may be, amongst other, preferred for the fusion to a GPCR as described herein.

In particular embodiments, the G protein peptidomimetic, in particular the G_{q/11} protein peptidomimetic, comprises a sequence of the structure (XII) or (XIII):

(XII)
(SEQ ID NO: 32)
FNDX₄KDIILQMNLRX₁₅X₁₆X₁₇X₁₈X₁₉
(XIII)
(SEQ ID NO: 33)
FAAVKDTILQLNLKX₁₅X₁₆X₁₇X₁₈X₁₉,

wherein X₁₄, X₁₅, X₁₆, X₁₇, X₁₈ and X₁₉ are as defined elsewhere herein.
G Protein Peptidomimetics with Additional Basic Amino Acids at the N-Terminus

In embodiments, the G protein peptidomimetic, in particular the G_{q/11} protein peptidomimetic, comprises at least one additional basic amino acid at its amino-terminus (N-terminus). Addition of one or more basic amino acids may improve the solubility of the G protein peptidomimetic. Addition of one or more basic amino acids may also facilitate cellular intake and uptake or penetration into cells. Such peptide comprising two or more such as up to 8 basic amino acids may also be referred to herein as a “cell-penetrating peptide (CPP)”, in particular a cationic or polycationic CPP.

The G protein peptidomimetics, in particular the G_{q/11} protein peptidomimetics, may comprise between 1 and 10, preferably between 1 and 8 such as 8, 7, 6, 5, 4, 3, 2, or 1 additional basic amino acids. In certain embodiments, the G protein peptidomimetic, in particular the G_{q/11} protein peptidomimetic, comprises between 1 and 3 additional basic amino acids. In certain embodiments, the G protein peptidomimetic, in particular the G_{q/11} protein peptidomimetic, comprises between 3 and 8 additional basic amino acids.

As used herein the term “basic amino acid” refers to an amino acid that is positively charged at physiological pH. Alternatively worded, the term “basic amino acid” as used herein refers to any amino acid that behaves as a Bronsted/Lowry and Lewis base. The term encompasses both natural and non-natural amino acids. Non-limiting examples of basic amino acids that can be added to the G protein peptidomimetic disclosed herein include lysine (K), histidine (H), arginine (R), D-arginine, hydroxylysine, ornithine, 2,4-diamino-butyric acid, (guanidino)-acetic acid or other (guanidino)alkyl-acetic acids.

In embodiments, the basic amino acid is selected from lysine (K), histidine (H) arginine (R) and D-arginine.

In particular embodiments, the G protein peptidomimetics, in particular the G_{q/11} protein peptidomimetics, described herein are modified by the addition of a single (K), a double (KK) or triple (KKK) lysine at their N-terminus, preferably a triple lysine.

In particular embodiments, the basic amino acid is selected from lysine (K), arginine (R) and D-arginine. In certain embodiments, the basic amino acid is D-arginine.

Optionally, said additional basic amino acid(s) may be linked to the G protein peptidomimetic, in particular the G_{q/11} protein peptidomimetic, via a spacer or linker, as known in the art. Non-limiting examples of suitable linkers or spacers include betaAla, Gly (repeats), aminohexanoic acid (Ahx), etc.

G Protein Peptidomimetics with Additional Modifications

Any of the peptides and peptidomimetics described herein can include various (chemical) modifications as long as the biological activity (e.g. the ability to stabilize a GPCR in an active conformational state) is not affected.

For example, any of the G protein peptidomimetics, in particular the G_{q/11} protein peptidomimetics, can be amidated (i.e. addition of an amide or substituted amide group) at its carboxy-terminus.

For example, any of the G protein peptidomimetics, in particular the G_{q/11} protein peptidomimetics, can be modified at its amino-terminus. Non-limiting examples of N-terminal modifications include acylation (e.g. acetyl, formyl, pyroglutamyl, fatty acids), alkylation, guanidinylation, attachment of urea, carbamate, sulfonamide, alkylamine, radioligand molecules (e.g. DOTA, NOTA, NODAGA), dyes and quencher molecules, etc. In particular embodiments, the G protein peptidomimetics, in particular the G_{q/11} protein peptidomimetic, comprises an N-terminal acylation. In further particular embodiments, the G protein peptidomimetics, in particular the G_{q/11} protein peptidomimetic comprise an N-terminal acetylation. In particular embodiments, the G protein peptidomimetics, in particular the G_{q/11} protein peptidomimetic comprises an N-terminal alkylation. In particular embodiments, the G protein peptidomimetics, in particular the G_{q/11} protein peptidomimetic comprises an N-terminal guanidinylation.

Other modifications can also be made to any of the G protein peptidomimetics, in particular the G_{q/11} protein peptidomimetics, described herein. For example, the peptide or peptidomimetic can be phosphorylated, glycosylated, PEGylated, lipidated, or any combination thereof.

Yet another modification may comprise the introduction of one or more detectable labels or other signal-generating groups or moieties, depending on the intended use of the labelled G protein peptidomimetic or G_{q/11} protein peptidomimetic. Suitable labels and techniques for attaching, using and detecting them will be clear to the skilled person, and for example include, but are not limited to, fluorescent labels (such as DY-647P1, Pacific Blue, Sulfocyanine 3 and Sulfocyanine 5, IRDye800, VivoTag800, fluorescein, isothiocyanate, rhodamine, phycoerythrin, phycocyanin, allophycocyanin, o-phthaldehyde, and fluorescamine and fluorescent metals such as Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogues), radio-isotopes, metals, metal chelates or metallic cations or other metals or metallic cations that are particularly suited for use in in vivo, in vitro or in situ diagnosis and imaging, as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholine esterase). Other suitable labels will be clear to the skilled person, and for example include moieties that can be detected using NMR or ESR spectroscopy. Such labelled G protein peptidomimetics or G_{q/11} protein peptidomimetics of the invention may for example be used for in vitro, in vivo or in situ assays (including immunoassays known per se such as ELISA, RIA, EIA and other “sandwich assays”, etc.) as well as in vivo diagnostic and imaging purposes, depending on the choice of the specific label. As will be clear to the skilled person, another modification may involve the introduction of a chelating group, for example to chelate one of the metals or metallic cations referred to above. Suitable chelating groups for example include, without limitation, 2,2′,2″-(10-(2-((2,5-dioxopyrrolidin-1-yl)oxy)-2-oxoethyl)-1,4,7,10-tetraazacyclododecane-1,4,7-triyl)triacetic acid (DOTA), 2,2′-(7-(2-((2,5-dioxopyrrolidin-1-yl)oxy)-2-oxoethyl)-1,4,7-triazonane-1,4-diyl)diacetic acid (NOTA), diethyl-enetriaminepentaacetic acid (DTPA) or ethylenediaminetetraacetic acid (EDTA). Yet another modification may comprise the introduction of a functional group that is one part of a specific binding pair, such as the biotin-(strept)avidin binding pair. Such a functional group may be used to link the G protein peptidomimetic or G_{q/11} protein peptidomimetic to another protein, polypeptide or chemical compound that is bound to the other half of the binding pair, i.e. through formation of the binding pair. For example, a G protein peptidomimetic, in particular a G_{q/11} protein peptidomimetic, as described herein may be conjugated to biotin, and linked to another protein, polypeptide, compound or carrier conjugated to avidin or streptavidin. For example, such a conjugated G protein peptidomimetic or G_{q/11} protein peptidomimetic may be used as a reporter, for example in a diagnostic system where a detectable signal-producing agent is conjugated to avidin or streptavidin. Such binding pairs may for example also be used to bind the G protein peptidomimetic or G_{q/11} protein peptidomimetic to a carrier, including carriers suitable for pharmaceutical purposes. Such binding pairs may also be used to link a therapeutically active agent to the G protein peptidomimetic, in particular the G_{q/11} protein peptidomimetic, of the invention.

In particular embodiments, the G protein peptidomimetic, in particular the G_{q/11} protein peptidomimetic, comprises a fluorescent label. Techniques for adding a fluorescent label or fluorophore to a peptide are well-known to the skilled person. For example, the fluorescent label or fluorophore may be incorporated at the N-terminal of the peptide, or react with a cysteine residue in or added to (the N-terminus of) a peptide. The fluorescent label or fluorophore may be directly added to the G protein peptidomimetic, in particular the Gq/11 protein peptidomimetic, or via a spacer or linker, as known in the art. Non-limiting examples of suitable linkers or spacers include betaAla, Gly (repeats), aminohexanoic acid (Ahx), etc.

Yet a further modification of the G protein peptidomimetics, in particular the G_{q/11} protein peptidomimetics, described herein, may be the addition of a cell-penetrating peptide (CPP) to facilitate penetration into a cell. Non-limiting examples of CPPs are cationic or polycationic CPPs, amphipathic CPPs and hydrophobic CPPs. As used herein, “cationic cell-penetrating peptides (CPPs)” or “polycationic CPPs” refer to cationic peptides of less than 30 amino acids such as from 5 to 30 amino acids, which comprise basic residues, preferably arginine and/or lysine, more preferably arginine. Without wishing to be bound by any theory, cationic CPPs may bind to negatively-charged groups in lipids and carbohydrates in the cell membrane due to their overall positive charge. Non-limiting examples of cationic CPPs include arginine-, D-arginine- or lysine-rich peptides (or poly-arginines, poly-D-arginines or poly-lysines) such as Arg₈(SEQ ID NO:36) and Arg₄ (SEQ ID NO:37 as described also elsewhere herein, and TAT peptide consisting of the sequence set forth in SEQ ID NO:38 (GRKKRRQRRRPPQ). As used herein, “amphipathic cell-penetrating peptides (CPPs)” refer to peptides varying from 5 to 30 amino acids in length that have alternating hydrophilic and hydrophobic residues, including Trp, Ile and Phe. Without wishing to be bound by any theory, hydrophobic residues within amphipathic CPPs may bind with hydrophobic lipid tails in the cell membrane. A non-limiting example of an amphipathic CPP is the RW9 (nona)peptide consisting of the sequence set forth in SEQ ID NO:39 (RRWWRRWRR). As used herein, “hydrophobic cell-penetrating peptides (CPPs)” refer to peptides varying from 5 to 30 amino acids in length of which the majority of the amino acids are hydrophobic residues. Without wishing to be bound by any theory, the hydrophobic residues may provide the hydrophobic CPP with the ability to cross cell membranes. A non-limiting example of a hydrophobic CPP is the peptide consisting of the sequence set forth in SEQ ID NO:40 (PFVYLI). In particular embodiments, the G protein peptidomimetic, in particular the G_{q/11} protein peptidomimetic, (additionally) comprises a CPP such as a polycationic CPP, an amphipathic CPP or a hydrophobic CPP. In particular embodiments, the G protein peptidomimetic, in particular the G_{q/11} protein peptidomimetic, is modified by addition of a polycationic CPP such as Arg₄ or Arg₈. In particular embodiments, the G protein peptidomimetic, in particular the G_{q/11} protein peptidomimetic, is modified by addition of an amphipathic CPP such as RW9 consisting of the sequence set forth in SEQ ID NO:39. In preferred embodiments, the CPP is added to the amino-terminus (N-terminus) of the G protein peptidomimetic, in particular the Gq/11 protein peptidomimetic. The CPP may be directly linked to the G protein peptidomimetic, in particular the Gq/11 protein peptidomimetic, or via a spacer or linker, as known in the art. Non-limiting examples of suitable linkers or spacers include betaAla, Gly (repeats), aminohexanoic acid (Ahx), etc. In embodiments, the G protein peptidomimetic, in particular the Gq/11 protein peptidomimetic, is modified by direct addition of a CPP at its N-terminus.

In particular embodiments, the G protein peptidomimetic, in particular the G_{q/11} protein peptidomimetic, is modified by addition of a CPP and a fluorescent label. In particular embodiments, the G protein peptidomimetic, in particular the Gq/11 protein peptidomimetic, is modified by addition of a CPP at its amino-terminus (N-terminus) and further by addition of a fluorescent label to said CPP motif. The fluorescent label or fluorophore may be added directly to the CPP or via a spacer or linker, as known in the art. Non-limiting examples of suitable linkers or spacers include betaAla, Gly (repeats), aminohexanoic acid (Ahx), etc.

### Variants of G Protein Peptidomimetics

The G protein peptidomimetics, in particular the G_{q/11} protein peptidomimetics, disclosed herein also encompass functional variants thereof. Functionally variant G protein peptidomimetics and G_{q/11} protein peptidomimetics include peptides and peptidomimetics having one or more conservative or non-conservative amino acid substitutions as compared to the sequences of the peptides and peptidomimetics described herein, but still retain substantially the same biological activity as the peptide or peptidomimetic described herein that does not have the substitution.

In particular, the terms “variant” of a peptide or peptidomimetic refers to peptides or peptidomimetics the sequence (i.e., amino acid sequence) of which is substantially identical (i.e., largely but not wholly identical) to the sequence of said recited peptide or peptidomimetic, e.g., at least about 80% identical or at least about 85% identical, e.g., preferably at least about 90% identical, e.g., at least 91% identical, 92% identical, more preferably at least about 93% identical, e.g., at least 94% identical, even more preferably at least about 95% identical, e.g., at least 96% identical, yet more preferably at least about 97% identical, e.g., at least 98% identical, and most preferably at least 99% identical. Preferably, a variant may display such degrees of identity to a recited peptide or peptidomimetic when the whole sequence of the recited peptide or peptidomimetic is queried in the sequence alignment (i.e., overall sequence identity). Also included among variants of a peptide or peptidomimetic are fusion products of said peptide or peptidomimetic with another, usually unrelated, peptide or peptidomimetic. Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250), for example using the published default settings or other suitable settings (such as, e.g., for the BLASTP algorithm: matrix=Blosum62, cost to open a gap=11, cost to extend a gap=1, expectation value=10.0, word size=3).

Amino acid substitutions may be generally based on the relative similarity of the amino acid side-chain substituents, for example, their hydrophobicity, hydrophilicity, charge, size and the like. Within the scope of the invention, conservative amino acid changes means an amino acid change at a particular position which may be of the same type as originally present; i.e. a hydrophobic amino acid exchanged for a hydrophobic amino acid, a basic amino acid for a basic amino acid, etc. Examples of conservative substitutions may include, without limitation, the substitution of non-polar (hydrophobic) residues such as isoleucine, valine, leucine or methionine for another, the substitution of one polar (hydrophilic) residue for another such as between arginine and lysine, between glutamine and asparagine, between threonine and serine, the substitution of one basic residue such as lysine, arginine or histidine for another, or the substitution of one acidic residue, such as aspartic acid or glutamic acid for another, the substitution of a branched chain amino acid, such as isoleucine, leucine, or valine for another, the substitution of one aromatic amino acid, such as phenylalanine, tyrosine or tryptophan for another. Examples of such conservative changes are well-known to the skilled artisan and are within the scope of the present invention.

Conservative substitution may also include the use of a chemically derivatized residue in place of a non-derivatized residue provided that the resulting peptide or peptidomimetic is a biologically functional equivalent to the peptides and peptidomimetics described herein.

Other substitutions that are contemplated herein are non-natural amino acids that are substituted for natural amino acids of the peptidomimetics described herein, so long as the peptidomimetic having substituted amino acid(s) retains substantially the same activity as the peptidomimetic in which amino acid(s) have not been substituted. Examples of non-natural amino acids include, but are not limited to, ornithine, citrulline, hydroxyproline, homoserine, phenylglycine, taurine, iodotyrosine, 2,4-diaminobutyric acid, α-amino isobutyric acid, 4-aminobutyric acid, 2-amino butyric acid, γ-amino butyric acid, ε-amino hexanoic acid, 6-amino hexanoic acid, 2-amino isobutyric acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general.

Furthermore, any of the amino acids in the protein can be of the D (dextrorotary) form or L (levorotary) form.

Illustrative G protein peptidomimetics, in particular G_{q/11} protein peptidomimetics, are shown in Table C.

TABLE C
Illustrative G_{q/11} protein peptidomimetics. ″[ ]″ denotes cyclic peptides.

| Compound | Sequence | SEQ ID NO: |
|---|---|---|
| SBL-GQ-01 | Ac-FAAVKDTILQLNLKEYNLV-OH | 1 |
| SBL-GQ-02 | Ac-KKKFAAVKDTILQLNLKEYNLV-OH | 2 |
| SBL-GQ-03 | Ac-FAAVKDTILQLNLKEYNChaV-OH | 3 |
| SBL-GQ-04 | Ac-KKKFAAVKDTILQLNLKEYNChaV-OH | 4 |
| SBL-GQ-05 | Ac-KKKFA[PraVKDAzk]ILQLNLKEYNLV-OH | 5 |
| SBL-GQ-06 | Ac-KKKFA[PraVKDAzk]ILQLNLKEYNChaV-OH | 6 |
| SBL-GQ-07 | Ac-FNDCKDIILQMNLREYNLV-OH | 7 |
| SBL-GQ-08 | Ac-KKKFNDCKDIILQMNLREYNLV-OH | 8 |
| SBL-GQ-09 | Ac-FNDCKDIILQMNLREYNChaV-OH | 9 |
| SBL-GQ-10 | Ac-KKKFNDCKDIILQMNLREYNChaV-OH | 10 |
| SBL-GQ-11 | Ac-KKKFN[PraCKDAzk]ILQMNLREYNLV-OH | 11 |
| SBL-GQ-12 | Ac-KKKFN[PraCKDAzk]ILQMNLREYNChaV-OH | 12 |

### Salts of the G Protein Peptidomimetics

Salts of the G protein peptidomimetics or the G_{q/11} protein peptidomimetics disclosed herein include those which are prepared with acids or bases, depending on the particular substituents present on the subject peptides and peptidomimetics described herein. Examples of a base addition salts include sodium, potassium, calcium, ammonium, or magnesium salt. Examples of acid addition salts include hydrochloric, hydrobromic, nitric, phosphoric, carbonic, sulphuric, and organic acids like acetic, trifluoroacetic, propionic, benzoic, succinic, fumaric, mandelic, oxalic, citric, tartaric, maleic, and the like.

### Functional Characterization

In preferred embodiments, the G protein peptidomimetics, in particular the G_{q/11} protein peptidomimetics, disclosed herein are “capable of stabilizing a GPCR in an active conformational state”.

The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, μ-sheet, among others), tertiary structure (e.g., the three dimensional folding of a polypeptide chain), and quaternary structure (e.g., interactions of a polypeptide chain with other protein subunits). Post-translational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labelling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993. A “specific conformational state” is any subset of the range of conformations or conformational states that a protein may adopt.

A “functional conformation” or a “functional conformational state”, as used herein, refers to the fact that proteins possess different conformational states having a dynamic range of activity, in particular ranging from no activity to maximal activity. It should be clear that “a functional conformational state” is meant to cover any conformational state of a GPCR, having any activity, including no activity; and is not meant to cover the denatured states of proteins. For example, a “basal conformational state” can be defined as a low energy state of the receptor in the absence of a ligand (e.g. effector molecules, agonists, antagonists, inverse agonists). An “active conformational state” of a GPCR as used herein refers to a spectrum of receptor conformations that allows signal transduction towards an intracellular effector system, including G protein dependent signalling and G protein-independent signalling (e.g. β-arrestin signalling). Typically, an active conformational state of a GPCR is in the presence of a ligand and an “active conformation” thus encompasses a range of ligand-specific conformations, including an agonist conformation, a partial agonist conformation or a biased agonist conformation.

The term “stabilizing” or “stabilized”, with respect to a functional conformational state of a GPCR, refers to an increased stability of a GPCR with respect to the structure (e.g. conformational state) and/or particular biological activity (e.g. intracellular signalling activity, ligand binding affinity, . . . ). In relation to increased stability with respect to structure and/or biological activity, this may be readily determined by either a functional assay for activity (e.g. Ca²⁺ release, cAMP generation or transcriptional activity, β-arrestin recruitment, . . . ) or ligand binding or by means of physical methods such as X-ray crystallography, NMR, or spin labelling, among other methods.

Within this context, a G protein peptidomimetic or a G_{q/11} protein peptidomimetic capable of stabilizing a GPCR in an active conformational state may also be referred to as a G protein peptidomimetic or a G_{q/11} protein peptidomimetic “capable of specifically or selectively binding to a GPCR in an active conformational state”. A binding agent, in particular a G protein peptidomimetic or a G_{q/11} protein peptidomimetic, that selectively binds to a specific conformation or conformational state of a GPCR generally refers to a binding agent that binds with a higher affinity to a GPCR in a subset of conformations or conformational states than to other conformations or conformational states that the GPCR may assume.

Within the context of the spectrum of conformational states of GPCRs, the terms “specifically bind” and “specific binding”, as used herein, refer to the ability of a G protein peptidomimetic or a G_{q/11} protein peptidomimetic as disclosed herein to preferentially recognize and/or bind to a particular conformational state of a GPCR as compared to another conformational state.

The term “affinity”, as used herein, refers to the degree to which a ligand or a binding agent (e.g. a G protein peptidomimetic or a G_{q/11} protein peptidomimetic) binds to a target protein so as to shift the equilibrium of target protein and ligand/binding agent toward the presence of a complex formed by their binding. Thus, for example, where a GPCR and a ligand are combined in relatively equal concentration, a ligand of high affinity will bind to the available antigen on the GPCR so as to shift the equilibrium toward high concentration of the resulting complex. The dissociation constant is commonly used to describe the affinity between a ligand or a binding agent and a target protein. Typically, the dissociation constant is lower than 10⁻⁵ M. Preferably, the dissociation constant is lower than 10⁻⁶ M, more preferably, lower than 10⁻⁷ M. Most preferably, the dissociation constant is lower than 10⁻⁸ M. Other ways of describing the affinity between a ligand or a binding agent and its target protein are the association constant (Kₐ), the inhibition constant (Kᵢ), or indirectly by evaluating the potency of ligands by measuring the half maximal inhibitory concentration (IC₅₀) or half maximal effective concentration (EC₅₀). It will be appreciated that within the scope of the present invention, the term “affinity” is used in the context of a binding agent, in particular a G protein peptidomimetic or a G_{q/11} protein peptidomimetic as disclosed herein, as well as in the context of a ligand or test compound that binds to a target GPCR.

Various methods may be used to determine specific binding (as defined herein before) between a G protein peptidomimetic or a G_{q/11} protein peptidomimetic and a target GPCR, including for example, enzyme linked immunosorbent assays (ELISA), flow cytometry, radioligand binding assays (also referred to as radioligand displacement assay or RLA), surface plasmon resonance assays, phage display, bimane fluorescence assay, and the like, which are common practice in the art and are further illustrated in the Example section.

A radioligand displacement assay can quantify the pharmacological stabilization of the GPCR in the active conformation by comparing the affinities of an agonist for the basal versus the active GPCR conformer. In particular, stabilization of a GPCR in an active conformational state or specific binding to a GPCR in an active conformational state by a G protein peptidomimetic or a G_{q/11} protein peptidomimetic as disclosed herein can be determined based on the shift in IC₅₀ value of a ligand, in particular an agonist more particularly an orthosteric agonist, which binds to the GPCR when tested in the presence and the absence of the G protein peptidomimetic or the G_{q/11} protein peptidomimetic. During the assay, the binding affinity of an orthosteric agonist for the receptor in presence and absence of the (allosteric) peptidomimetic is determined. Therefore, the GPCR, e.g. embedded in membrane extracts, is incubated with a radioligand and different concentrations of the agonist. By increasing the agonist concentration, radioligand will be displaced by the agonist. A leftward shift of the curve in the presence of the peptidomimetic is indicative of a peptidomimetic capable of stabilizing a GPCR in an active conformational state. A “shift” in IC₅₀ value or IC₅₀ shift is defined herein as the IC₅₀ ratio of a ligand, in particular an agonist, more particularly an orthosteric agonist, which binds to the GPCR in the absence of a G protein peptidomimetic or a G_{q/11} protein peptidomimetic relative to the presence of the G protein peptidomimetic or the G_{q/11} protein peptidomimetic, or the IC₅₀ value of a ligand, in particular an agonist, for binding to the GPCR in the absence of a G protein peptidomimetic or a G_{q/11} protein peptidomimetic divided by the IC₅₀ value of the ligand in the presence of the G protein peptidomimetic or the G_{q/11} protein peptidomimetic in the same preparation, wherein said IC₅₀ values are determined in a radioligand binding assay or radioligand displacement assay (RLA) as known to the skilled person. For example, a human muscarinic acetylcholine 1 receptor (M1R) radioligand binding assay using ³H—N-methyl scopolamine (³H-NMS) as the radioligand and increasing concentrations of acetylcholine chloride (agonist) as cold competitor may be used.

In particular embodiments, the G protein peptidomimetics, in particular the G_{q/11} protein peptidomimetics, induce a shift in IC₅₀ value of more than 5, preferably more than 10, more preferably more than 20, even more preferably more than 30, wherein said shift is determined in a radioligand binding assay wherein the GPCR is human M1P, the radioligand is ³H-NMS, and the unlabelled ligand is acetylcholine chloride.

Another assay that can be used to analyse conformational changes associated with GPCR activation and/or to identify G protein peptidomimetics that are capable of stabilizing a GPCR in an active conformational state is the bimane fluorescence assay or bimane assay. This assay works through labelling of a cysteine residue in the lower part of the TM6 of a GPCR with a bimane-fluorophore (monobromobimane (MB)). Binding of an agonist to the GPCR causes a conformational change and outward movement of TM6 that places bimane in a more solved-exposed position, which will alter its maximum emission wavelength. In particular, increasing concentrations of agonist result in a concentration-dependent red-shift of the maximum emission wavelength (λmax) of the bimane-fluorophore probe. In the presence of a G protein or an active state stabilizing G protein peptidomimetic λmax may increase further. For example, a bimane labelled ghrelin receptor (such as a cysmin mutant (i.e. mutant with minimal cysteines) of human ghrelin receptor (also referred to herein as growth hormone secretagogue receptor (GHSR) (e.g. the mutant as described in Damian et al. (2021) wherein monobromobimane (MB) fluorescent probe is attached to Cys²⁵⁵) and ghrelin receptor agonist JMV1843 may be used.

In particular embodiments, the G protein peptidomimetics, in particular the G_{q/11} protein peptidomimetics, induce a maximum emission wavelength (Amax) of more than 476 nm, preferably more than 477 nm, more preferably more than 478 nm, wherein said Amax is determined in a bimane fluorescence assay wherein the GPCR is human ghrelin receptor and the agonist is JMV1843.

“G protein-coupled receptors”, or “GPCRs”, as used herein, are polypeptides that share a common structural motif, having seven regions of between 22 to 24 hydrophobic amino acids that form seven alpha helices, each of which spans the membrane. Each span is identified by number, i.e., transmembrane-1 (TM1), transmembrane-2 (TM2), etc. The transmembrane helices are joined by regions of amino acids between transmembrane-2 and transmembrane-3, transmembrane-4 and transmembrane-5, and transmembrane-6 and transmembrane-7 on the exterior, or “extracellular” side, of the cell membrane, referred to as “extracellular” regions 1, 2 and 3 (EC1, EC2 and EC3), respectively. The transmembrane helices are also joined by regions of amino acids between transmembrane-1 and transmembrane-2, transmembrane-3 and transmembrane-4, and transmembrane-5 and transmembrane-6 on the interior, or “intracellular” side, of the cell membrane, referred to as “intracellular” regions 1, 2 and 3 (IC1, IC2 and IC3), respectively. The “carboxy” (“C”) terminus of the receptor lies in the intracellular space within the cell, and the “amino” (“N”) terminus of the receptor lies in the extracellular space outside of the cell. Any of these regions are readily identifiable by analysis of the primary amino acid sequence of a GPCR.

GPCRs can be grouped on the basis of sequence homology into several distinct families. Although all GPCRs have a similar architecture of seven membrane-spanning α-helices, the different families within this receptor class show no sequence homology to one another, thus suggesting that the similarity of their transmembrane domain structure might define common functional requirements. A comprehensive view of the GPCR repertoire was possible when the first draft of the human genome became available. Fredriksson and colleagues divided 802 human GPCRs into families on the basis of phylogenetic criteria. This showed that most of the human GPCRs can be found in five main families, termed Rhodopsin, Adhesion, Secretin, Glutamate, Frizzled/Taste2 (Fredriksson et al., 2003). Members of the Rhodopsin family (corresponding to class A (Kolakowski, 1994)) or Class 1 (Foord et al (2005) in older classification systems)) only have small extracellular loops and the interaction of the ligands occurs with residues within the transmembrane cleft. This is by far the largest group (>90% of the GPCRs) and contains receptors for odorants, small molecules such as catecholamines and amines, (neuro)peptides and glycoprotein hormones. Rhodopsin, a representative of this family, is the first GPCR for which the structure has been solved. P2AR, the first receptor interacting with a diffusible ligand for which the structure has been solved (Rosenbaum et al, 2007) also belongs to this family. Based on phylogenetic analysis, class B GPCRs or Class 2 (Foord et al, 2005) receptors have recently been subdivided into two families: adhesion and secretin (Fredriksson et al., 2003). Adhesion and secretin receptors are characterized by a relatively long amino terminal extracellular domain involved in ligand-binding. Little is known about the orientation of the transmembrane domains, but it is probably quite different from that of rhodopsin. Ligands for these GPCRs are hormones, such as glucagon, secretin, gonadotropin-releasing hormone and parathyroid hormone. The glutamate family receptors (Class C or Class 3 receptors) also have a large extracellular domain, which functions like a “Venus fly trap” since it can open and close with the agonist bound inside. Family members are the metabotropic glutamate, the Ca²⁺-sensing and the γ-aminobutyric acid (GABA)-B receptors.

GPCRs can also be classified based on the G protein to which they are coupled. Particular non-limiting examples are provided in Table A provided elsewhere herein.

In embodiments, the GPCR that is stabilized in an active conformational state by the G protein peptidomimetic, in particular the G_{q/11} protein peptidomimetic, disclosed herein is a G_{q/11} protein-coupled receptor. Non-limiting examples of G_{q/11} protein-coupled receptors include muscarinic acetylcholine receptor 1 (M1R), growth hormone secretagogue receptor or ghrelin receptor (GHSR), histamine 1 receptor (H1R) and 5-hydroxytryptamine 2A receptor (5-HT_{2A}R).

The human muscarinic acetylcholine receptor 1 (M1R) sequence can be found under/corresponds with or to UniProtKB accession: P11229, version P11229-1, and is also defined herein as SEQ ID NO: 15.

The human histamine 1 receptor (H1R) sequence can be found under/corresponds with or to UniProtKB accession: P35367, version P35367-1, and is also defined herein as SEQ ID NO: 16.

The human 5-hydroxytryptamine 2A receptor (5-HT_{2A}R) can be found under/corresponds with or to UniProtKB accession: P28223, version P28223-1, and is also defined herein as SEQ ID NO: 17.

In particular embodiments, the GPCR that is stabilized in an active conformational state by the G protein peptidomimetic, in particular the G_{q/11} protein peptidomimetic, is muscarinic acetylcholine receptor 1 (M1R) or ghrelin receptor (GHSR).

The GPCR that is stabilized in an active conformational state by the G protein peptidomimetic, in particular the G_{q/11} protein peptidomimetic, may be naturally occurring or non-naturally occurring (i.e., altered by man). The term “naturally-occurring”, as used herein, means a GPCR that is naturally produced. In particular, wild type polymorphic variants and isoforms of GPCRs, as well as orthologues across different species are examples of naturally occurring proteins. Thus, such GPCRs are found in nature. The term “non-naturally occurring”, as used herein, means a GPCR that is not naturally-occurring. In certain circumstances, it may be advantageous that the GPCR is a non-naturally occurring protein. For example, and for illustration purposes only, some protein engineering without or only minimally affecting ligand binding affinity might be performed to increase the probability of obtaining crystals of a GPCR stabilized in an active conformational state by a G protein peptidomimetic, in particular a G_{q/11} protein peptidomimetic, disclosed herein. Or, alternatively or additionally, to increase cellular expression levels of a GPCR, or to increase the stability, one might also consider introducing certain mutations in the GPCR of interest. Non-limiting examples of non-naturally occurring GPCRs include, without limitation, GPCRs that have been made constitutively active through mutation, GPCRs with a loop deletion, GPCRs with an N- and/or C-terminal deletion, GPCRs with a substitution, an insertion or addition, or any combination thereof, in relation to their amino acid or nucleotide sequence, or other variants of naturally-occurring GPCRs. Also comprised within the scope of the present invention are target GPCRs comprising a chimeric or hybrid GPCR, for example a chimeric GPCR with an N- and/or C-terminus from one GPCR and loops of a second GPCR, or comprising a GPCR fused to a moiety.

The nature of the GPCR is not critical to the invention and can be from any organism including a fungus (including yeast), nematode, virus, insect, plant, bird (e.g. chicken, turkey), reptile or mammal (e.g., a mouse, rat, rabbit, hamster, gerbil, dog, cat, goat, pig, cow, horse, whale, monkey, camelid, or human). Preferably, the GPCR is of mammalian origin, even more preferably of human origin.

### Fusion Polypeptides

The G protein peptidomimetics, in particular the G_{q/11} protein peptidomimetics, disclosed herein can be fused to the GPCR that they can stabilize in an active conformational state, optionally through use of a linker. In this way the constitutive stabilization of a unique active conformation of the GPCR can be obtained through an intramolecular reaction of both moieties. One key advantage of the fusion polypeptides disclosed herein is that a defined 1:1 stoichiometry of GPCR to G protein peptidomimetic is ensured in a single protein, forcing the physical proximity of the fusion partners, while maintaining the properties of the G protein peptidomimetic to stabilize the receptor in an active conformational state. It is thus particularly envisaged that the fusion polypeptides described herein comprise a GPCR moiety that is stabilized in an active conformation upon binding of the G protein peptidomimetic moiety in an intramolecular reaction, preferably without the need for an additional ligand.

Accordingly, an aspect relates to a fusion molecule or fusion polypeptide comprising i) a GPCR as defined herein and ii) a G protein peptidomimetic, in particular a G_{q/11} protein peptidomimetic, as disclosed herein that is capable of stabilizing said GPCR in an active conformational state. The G protein peptidomimetic is fused to the GPCR either directly or through a linker.

The terms “fusion polypeptide” or “fusion protein” are used interchangeably herein and refer to a protein that comprises at least two separate and distinct (poly)peptide components that may or may not originate from the same protein. The (poly)peptide components, while typically unjoined in their native state, are joined by their respective amino and carboxyl termini through a peptide linkage to form a single continuous polypeptide. The term “fused to”, and other grammatical equivalents, when referring to a fusion polypeptide (as defined herein) refers to any chemical or recombinant mechanism for linking two or more (poly)peptide components. The fusion of the two or more (poly)peptide components may be a direct fusion of the sequences or it may be an indirect fusion, e.g. with intervening amino acid sequences or linker sequences.

The way the different moieties that form part of the fusion polypeptides as described, in particular the GPCR and the G protein peptidomimetic, are fused to each other will typically depend on both the type of GPCR and the characteristics of the G protein peptidomimetic (e.g. linear or stapled G protein peptidomimetic).

As is known by the person skilled in the art, GPCRs are characterized by an extracellular N-terminus, followed by seven transmembrane α-helices connected by three intracellular and three extracellular loops, and finally an intracellular C-terminus. In preferred embodiments, the G protein peptidomimetic, in particular the G_{q/11} protein peptidomimetic, is fused to the C-terminus of the GPCR. The G protein peptidomimetic or G_{q/11} protein peptidomimetic will preferably be fused with its N-terminal end to the C-terminal end of the GPCR.

Further, the fusion may be a direct fusion of the sequences or it may be an indirect fusion, e.g. with intervening amino acid sequences or linker sequences.

Linker molecules or linkers may be peptides of 1 to 200 amino acids length, and are typically, but not necessarily, chosen or designed to be unstructured and flexible. For instance, one can choose amino acids that form no particular secondary structure. Or, amino acids can be chosen so that they do not form a stable tertiary structure. Or, the amino acid linkers may form a random coil. Such linkers include, but are not limited to, synthetic peptides rich in Gly, Ser, Thr, Gln, Glu or further amino acids that are frequently associated with unstructured regions in natural proteins. IUPred: web server for the prediction of intrinsically unstructured regions of proteins based on estimated energy content. Non-limiting examples include (GₓS_{z})_{b} wherein x and z are independently chosen integers from 0 to 9 one of them having at least a value of 1 and wherein b is an integer from 1 to 9. Preferably, the amino acid linker sequence has a low susceptibility to proteolytic cleavage and does not interfere with the biological activity of the fusion polypeptide. Hence, a suitable linker should not provide sterical hindrance or impede proper folding of the functional portion of either the G protein peptidomimetic or the GPCR.

A person skilled in the art will know how to design a fusion construct. For general methods relating to the present disclosure, reference is made inter alia to well-known textbooks, including e.g. “Molecular Cloning: A Laboratory Manual, 4^{th} Ed.” (Green and Sambrook et al., 2012, Cold Spring Harbor Laboratory Press)”. A convenient means for linking or fusing two (poly)peptides is by expressing them as a fusion protein from a recombinant nucleic acid molecule, which comprises a first polynucleotide encoding a first (poly)peptide operably linked to a second polynucleotide encoding the second (poly)peptide. This method is particularly preferably for G protein peptidomimetics and G_{q/11} protein peptidomimetics disclosed herein that have a peptide backbone consisting of naturally occurring amino acids, or peptidomimetics that consist of naturally occurring amino acids. Otherwise, the (poly)peptides comprised in a fusion protein can be linked through peptide bonds that result from chemoenzymatic methods. In case the G protein peptidomimetic and the GPCR moiety are linked using chemoenzymatic methods for protein modification, the linker moiety may exist of different chemical entities, depending on the enzymes or the synthetic chemistry that is used to produce the covalent chimer in vivo or in vitro.

### Complex

In an aspect, the invention provides a complex comprising a GPCR as defined herein and a G protein peptidomimetic, in particular a G_{q/11} protein peptidomimetic, as disclosed herein that specifically binds to said GPCR. In embodiments, the complex may further comprise at least one other receptor ligand.

A related aspect provides a complex comprising a fusion polypeptide disclosed herein and at least one other receptor ligand.

As used herein, the term “ligand” means a molecule that specifically binds to a GPCR. A ligand may be, without the purpose of being limitative, a polypeptide, a peptide, a peptidomimetic, a lipid, a small molecule, an antibody, an antibody fragment, a nucleic acid, a carbohydrate. A ligand may be synthetic or naturally occurring. A ligand includes a “native ligand” which is a ligand that is an endogenous, natural ligand for a native GPCR. Within the context of the present invention, a ligand may bind to a GPCR, either intracellularly or extracellularly. An “orthosteric ligand” as used herein, refers to a ligand that binds to the active site of a GPCR. Orthosteric ligands are further classified according to their efficacy or in other words to the effect they have on signalling through a specific pathway. As used herein, an “agonist” refers to a ligand that, by binding a receptor protein, increases the receptor's signalling activity. Full agonists are capable of maximal protein stimulation; partial agonists are unable to elicit full activity even at saturating concentrations. Partial agonists can also function as “blockers” by preventing the binding of more robust agonists. An “antagonist”, also referred to as a “neutral antagonist”, refers to a ligand that binds a receptor without stimulating any activity. An “antagonist” is also known as a “blocker” because of its ability to prevent binding of other ligands and, therefore, block agonist-induced activity. Further, an “inverse agonist” refers to an antagonist that, in addition to blocking agonist effects, reduces a receptor's basal or constitutive activity below that of the unliganded protein. Ligands as used herein may also be “biased ligands” (also known as “biased agonists” or “functionally selective agonists”) with the ability to selectively stimulate a subset of a receptor's signalling activities, for example in the case of GPCRs the selective activation of G-protein or β-arrestin function. More particularly, ligand bias can be an imperfect bias characterized by a ligand stimulation of multiple receptor activities with different relative efficacies for different signals (non-absolute selectivity) or can be a perfect bias characterized by a ligand stimulation of one receptor protein activity without any stimulation of another known receptor protein activity. Another kind of ligands is known as allosteric regulators. “Allosteric regulators” or otherwise “allosteric modulators”, “allosteric ligands” or “effector molecules”, as used herein, refer to ligands that bind at an allosteric site (that is, a regulatory site physically distinct from the protein's active site) of a GPCR. In contrast to orthosteric ligands, allosteric modulators are non-competitive because they bind receptor proteins at a different site and modify their function even if the endogenous ligand also is binding. Allosteric regulators that enhance the protein's activity are referred to herein as “allosteric activators” or “positive allosteric modulators” (PAMs), whereas those that decrease the protein's activity are referred to herein as “allosteric inhibitors” or otherwise “negative allosteric modulators” (NAMs).

In particular embodiments, the ligand in the complexes described herein may be a “conformation-selective ligand” or “conformation-specific ligand”, meaning that such a ligand binds the GPCR in a conformation-selective manner. A conformation-selective ligand binds with a higher affinity to a particular conformation of the GPCR than to other conformations the GPCR may adopt. In further particular embodiments, the ligand is an active conformation-selective ligand and the GPCR is an active conformational state in the complex described herein.

In embodiments, the ligand is an agonist (e.g. a partial agonist or full agonist) and the GPCR is in an active conformational state. The ligand may also be an inverse agonist, an antagonist or a biased ligand. Ligands also include allosteric modulators, potentiators, enhancers, negative allosteric modulators and inhibitors.

As a non-limiting example, a stable complex as described herein may be purified by size exclusion chromatography.

The complexes described herein may be crystalline. So, a crystal of the complex is also provided herein, as well as methods of making said crystal, which are described in greater detail below. Preferably, a crystalline form of a complex as described herein and a receptor ligand is envisaged.

### Compositions

The fusion polypeptides and complexes described herein may be in a solubilized form, such as in a detergent. Alternatively, the fusion polypeptide or complex may be immobilized to a solid support. Non-limiting examples of solid supports as well as methods and techniques for immobilization are well known to the skilled person. Yet alternatively, the fusion polypeptide or complex may be in a cellular composition, including an organism, a tissue, a cell, a cell line, or in a membrane composition or liposomal composition derived from said organism, tissue, cell or cell line. Examples of membrane or liposomal compositions include, but are not limited to organelles, membrane preparations, viruses, virus like lipoparticles, and the like. It will be appreciated that a cellular composition, or a membrane-like or liposomal composition may comprise natural or synthetic lipids.

Accordingly, the present invention also relates to compositions comprising a fusion polypeptide or a complex as described herein. Particular embodiments relate to a membrane or liposomal composition comprising a fusion polypeptide or a complex as described herein. Membrane compositions may be derived from a tissue, cell or cell line and include organelles, membrane extracts or fractions thereof, VLPs, viruses, and the like, as long as sufficient functionality of the fusion polypeptides and complexes is retained.

### Expression Systems

Further disclosed herein is a nucleic acid molecule comprising one or more nucleic acid sequences encoding a G protein peptidomimetic, in particular a G_{q/11} protein peptidomimetic, of the invention.

Also disclosed herein is a nucleic acid molecule comprising a nucleic acid sequence encoding a fusion polypeptide of the invention.

Further disclosed herein are expression vectors comprising nucleic acid sequences encoding a G protein peptidomimetic, in particular a G_{q/11} protein peptidomimetic, or fusion polypeptide as described herein, as well as host cells expressing such expression vectors.

In certain embodiments, the expression vector encodes a cleavable concatenation of the G protein peptidomimetic, optionally separated by a protease cleavage site sequence.

It is evident that further regulatory sequences may be part of the expression vector such as but not limited to promoters, enhancers, selection markers, origins of replication, linker sequences, polyA sequences, and degradation sequences. The term “selection marker” as used herein is to be interpreted in accordance to its generally accepted meaning in the art, i.e. a gene that allow for artificial selection of cells comprising (a certain amount of concentration of) the expression vector(s) carrying the selection marker. Suitable selection markers include prokaryotic or eukaryotic antibiotic resistance genes or fluorescent proteins. In certain embodiments wherein the G protein peptidomimetic and GPCR are encoded by a distinct expression vector, each expression vector can be construed in order to express a separate selection marker. The selection marker(s) may be fused to the GPCR and/or the G protein peptidomimetic or may be expressed as separate moieties. In the latter embodiments, expression of the selection marker(s) and the GPCR/G protein peptidomimetic may be governed (i.e. regulated) by a single promoter or by distinct promoters. In embodiments where expression of the selection marker and the GPCR and/or G protein peptidomimetic is regulated by a single promoter, the different moieties may still be expressed as separate elements by inclusion of one or more e.g. internal ribosomal entry sites (IRES) sequences or alternatively one or more 2A self-cleaving peptide sequences. Hence, bicistronic and multicistronic expression vectors are envisaged by the inventors and part of the scope of the invention.

Suitable expression systems include constitutive and inducible expression systems in bacteria or yeasts, virus expression systems, such as baculovirus, semliki forest virus and lentiviruses, or transient transfection in insect or mammalian cells. The cloning and/or expression of the G protein peptidomimetics and fusion polypeptides can be done according to techniques known by the skilled person in the art. The expression of the GPCR and/or G protein peptidomimetic may be governed by a constitutive promoter sequence or an inducible promoter sequence. Non-limiting examples of inducible expression systems are the tetracycline- or doxycycline-induced Tet-On and Tet-off expression systems (Gossen et al. 1995 PNAS 5547:5551, and Gossen et al. 1995 Science 1766:1769).

The “host cell” can be of any prokaryotic or eukaryotic organism. Preferably, the host cell is a eukaryotic cell and can be of any eukaryotic organism, but in particular embodiments yeast, plant, mammalian and insect cells are envisaged. The nature of the cells used will typically depend on the ease and cost of producing the G protein peptidomimetics and fusion polypeptides, the desired glycosylation properties, the origin of the fusion polypeptide, the intended application, or any combination thereof. Mammalian cells may for instance be used for achieving complex glycosylation, but it may not be cost-effective to produce proteins in mammalian cell systems. Plant and insect cells, as well as yeast typically achieve high production levels and are more cost-effective, but additional modifications may be needed to mimic the complex glycosylation patterns of mammalian proteins. Yeast cells are often used for expression of proteins because they can be economically cultured, give high yields of (medium-secreted) protein, and when appropriately modified are capable of producing proteins having suitable glycosylation patterns. Further, yeast offers established genetics allowing for rapid transformations, tested protein localization strategies, and facile gene knock-out techniques. Insect cells are also an attractive system to express GPCRs because insect cells offer an expression system without interfering with mammalian GPCR signalling. Eukaryotic cell or cell lines for protein production are well known in the art, including cell lines with modified glycosylation pathways, and non-limiting examples will be provided hereafter.

Exemplary animal or mammalian host cells suitable for harboring, expressing, and producing proteins such as the G protein peptidomimetics and fusion polypeptides disclosed herein, for subsequent isolation and/or purification include Chinese hamster ovary cells (CHO), such as CHO-K1 (ATCC CCL-61), DG44 (Chasin et al., 1986; Kolkekar et al., 1997), CHO-K1 Tet-On cell line (Clontech), CHO designated ECACC 85050302 (CAMR, Salisbury, Wiltshire, UK), CHO clone 13 (GEIMG, Genova, IT), CHO clone B (GEIMG, Genova, IT), CHO-K1/SF designated ECACC 93061607 (CAMR, Salisbury, Wiltshire, UK), RR-CHOK1 designated ECACC 92052129 (CAMR, Salisbury, Wiltshire, UK), dihydrofolate reductase negative CHO cells (CHO/-DHFR, Urlaub and Chasin, 1980), and dp12.CHO cells (U.S. Pat. No. 5,721,121); monkey kidney CV1 cells transformed by SV40 (COS cells, COS-7, ATCC CRL-1651); human embryonic kidney cells (e.g., 293 cells, or 293T cells, or 293 cells subcloned for growth in suspension culture, Graham et al., 1977, J. Gen. Virol., 36:59, or GnTI KO HEK293S cells, Reeves et al. 2002); baby hamster kidney cells (BHK, ATCC CCL-10); monkey kidney cells (CV1, ATCC CCL-70); African green monkey kidney cells (VERO-76, ATCC CRL-1587; VERO, ATCC CCL-81); mouse sertoli cells (TM4, Mather, 1980, Biol. Reprod., 23:243-251); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TRI cells (Mather, 1982); MCR 5 cells; FS4 cells. Preferably, the cells are mammalian cells selected from Hek293 cells or COS cells.

Exemplary non-mammalian cell lines include, but are not limited to, insect cells, such as Sf9 cells/baculovirus expression systems (e.g. review Jarvis, Virology Volume 310, Issue 1, 25 May 2003, Pages 1-7), plant cells such as tobacco cells, tomato cells, maize cells, algae cells, or yeasts such as Saccharomyces species, Schizosaccharomyces species, Hansenula species, Yarrowia species or Pichia species. According to particular embodiments, the eukaryotic cells are yeast cells from a Saccharomyces species (e.g. Saccharomyces cerevisiae), Schizosaccharomyces sp. (for example Schizosaccharomyces pombe), a Hansenula species (e.g. Hansenula polymorpha), a Yarrowia species (e.g. Yarrowia lipolytica), a Kluyveromyces species (e.g. Kluyveromyces lactis), a Pichia species (e.g. Pichia pastoris), or a Komagataella species (e.g. Komagataella pastoris).

Transfection of target cells (e.g. mammalian cells) can be carried out following principles outlined by Sambrook and Russel (Molecular Cloning, A Laboratory Manual, 3^{rd} Edition, Volume 3, Chapter 16, Section 16.1-16.54). In addition, viral transduction can also be performed using reagents such as adenoviral vectors. Selection of the appropriate viral vector system, regulatory regions and host cell is common knowledge within the level of ordinary skill in the art. The resulting transfected cells are maintained in culture or frozen for later use according to standard practices.

### Applications

The above described G protein peptidomimetics and G_{q/11} protein peptidomimetics as well as the complexes and fusion polypeptides comprising these G protein peptidomimetics and G_{q/11} protein peptidomimetics are particularly useful in a variety of contexts and applications. For example, and without limitation, (1) for capturing and/or purification of a GPCR whereby upon binding, the G protein peptidomimetic, in particular the G_{q/11} protein peptidomimetic, maintains the receptor in a particular conformation, in particular an active conformation; (2) for co-crystallization studies and high-resolution structural analysis of a GPCR in complex with the G protein peptidomimetic, in particular the G_{q/11} protein peptidomimetic, and optionally additionally bound to another conformation-selective receptor ligand; (3) for ligand characterization, compound screening, and (structure-based) drug discovery; (4) as allosteric modulator of GPCR signalling; and/or (5) as a biosensor, e.g. for detecting conformational changes of a GPCR, for assessing the localization and/or trafficking of a GPCR, and/or for investigating a GPCR signalling pathway, all of which will be described into further detail below.

### Capturing, Separation and Purification Methods for GPCR in a Functional Conformation

In an aspect, the invention provides a method for capturing and/or purifying a GPCR in a functional conformation, preferably an active conformation, by making use of any of the above described G protein peptidomimetics and G_{q/11} protein peptidomimetics. Capturing and/or purifying a receptor in a particular conformation such as an active conformation will allow amongst others subsequent crystallization, ligand characterization, compound screening, immunizations, etc.

Thus, the invention relates to the use, preferably an in vitro or ex vivo use, of a G protein peptidomimetic, in particular a G_{q/11} protein peptidomimetic, as described herein to capture a GPCR in a functional conformation, in particular an active conformation. Optionally, but not necessarily, capturing of a GPCR in an active conformation may include capturing a GPCR in complex with another conformation-selective receptor ligand (e.g. an orthosteric ligand, an allosteric ligand, a natural binding partner such as an arrestin, and the like).

In accordance, the invention also provides a method of capturing a GPCR in a functional conformation, in particular an active conformation, said method comprising the steps of:
a) bringing a G protein peptidomimetic, in particular a G_{q/11} protein peptidomimetic, as described herein into contact with a GPCR, and
b) allowing the G protein peptidomimetic to specifically bind to the GPCR, whereby GPCR is captured in a functional conformation, in particular an active conformation.

In embodiments, the invention also envisages a method of capturing a GPCR in a functional conformation, in particular an active conformation, said method comprising the steps of:
a) applying a solution containing GPCR in a plurality of conformations to a solid support possessing an immobilized G protein peptidomimetic, in particular a G_{q/11} protein peptidomimetic, as described herein, and
b) allowing the G protein peptidomimetic to specifically bind to the GPCR, whereby the GPCR is captured in a functional conformation, in particular an active conformation and
c) optionally removing weakly bound or unbound molecules.

It will be appreciated that any of the methods as described above may further comprise the step of isolating the complex formed in step (ii) of the above described methods, said complex comprising the G protein peptidomimetic and the GPCR in a particular conformation. Suitable techniques for isolating/purifying GPCRs include, without limitation, affinity-based methods such as affinity chromatography, affinity purification, immunoprecipitation, protein detection, immunochemistry, surface-display, size exclusion chromatography, ion exchange chromatography, amongst others, and are all well-known in the art.

### Crystallography and Applications in Structure-Based Drug Design

The G protein peptidomimetics, in particular the G_{q/11} protein peptidomimetics, disclosed herein are particularly useful in X-ray crystallography of GPCRs and applications thereof in structure-based drug design.

Agonist-bound receptor crystals may provide three-dimensional representations of the active states of GPCRs, which structures can help clarifying the conformational changes connecting the ligand-binding and G protein-interaction sites, and lead to more precise mechanistic hypotheses and eventually new therapeutics. Given the conformational flexibility inherent to ligand-activated GPCRs, stabilizing such a state, e.g. for crystal formation, is not easy. Such efforts can benefit from the stabilization of the agonist-bound receptor conformation by the addition of binding agents that are specific for an active conformational state of the receptor. It is thus a particular advantage of the G protein peptidomimetics, in particular the G_{q/11} protein peptidomimetics, that upon binding to the GPCR, they can stabilize the receptor in an active conformation, thereby reducing its conformational flexibility and increasing its polar surface, facilitating the crystallization of a receptor:G protein peptidomimetic complex. The G protein peptidomimetics, in particular the G_{q/11} protein peptidomimetics, of the present invention are therefore valuable tools to increase the probability of obtaining well-ordered crystals by minimizing the conformational heterogeneity in the target GPCR.

The so-obtained crystals will also be of great advantage to help guide drug discovery. Especially methods for acquiring structures of receptors bound to lead compounds that have pharmacological or biological activity and whose chemical structure is used as a starting point for chemical modifications in order to improve potency, selectivity, or pharmacokinetic parameters are very valuable and are provided herein. Persons of ordinary skill in the art will recognize that the G protein peptidomimetics, in particular the G_{q/11} protein peptidomimetics, disclosed herein are particularly suited for co-crystallization of receptor:G protein peptidomimetic with lead compounds that are selective for the druggable conformation induced by the G protein peptidomimetic because this G protein peptidomimetic is able to substantially increase the affinity of conformation-selective receptor ligands.

Thus, it is envisaged herein to use the G protein peptidomimetics, in particular the G_{q/11} protein peptidomimetics, disclosed herein for crystallization purposes. Advantageously, crystals can be formed of a complex of a G protein peptidomimetic, in particular a G_{q/11} protein peptidomimetic, as disclosed herein and a GPCR to which the G protein peptidomimetic specifically binds (as disclosed herein), wherein the receptor is trapped in a particular receptor conformation, more particularly a therapeutically relevant receptor conformation (e.g. an active conformation). The G protein peptidomimetic will also reduce the flexibility of extracellular regions upon binding the receptor to grow well-ordered crystals.

Accordingly, provided herein is the use of G protein peptidomimetics, in particular G_{q/11} protein peptidomimetics, as described herein for crystallizing a complex of a G protein peptidomimetic and a GPCR to which the G protein peptidomimetic can specifically bind, and eventually to solve the structure of the complex. Particular embodiments relate to crystallization of a complex of a G protein peptidomimetic, in particular a G_{q/11} protein peptidomimetic, as described herein, a GPCR to which the G protein peptidomimetic will specifically bind, and another conformation-selective receptor ligand (as defined hereinbefore).

In accordance, provided herein is a method of crystallizing a complex of a G protein peptidomimetic, in particular a G_{q/11} protein peptidomimetic, and a GPCR to which the G protein peptidomimetic can specifically bind and optionally determining the crystal structure of a GPCR in a functional conformation, in particular an active conformation, the method comprising the steps of:
a) providing a G protein peptidomimetic, in particular a G_{q/11} protein peptidomimetic, as described herein and a GPCR to which the G protein peptidomimetic can specifically bind, and optionally a receptor ligand, and
b) allowing the formation of a complex of the G protein peptidomimetic, the GPCR and optionally a receptor ligand,
c) crystallizing said complex of step b) to form a crystal, and
d) optionally obtaining the atomic coordinates of the crystal.

“Crystal” or “crystalline structure”, as used herein, refers to a solid material, whose constituent atoms, molecules, or ions are arranged in an orderly repeating pattern extending in all three spatial dimensions. The process of forming a crystalline structure from a fluid or from materials dissolved in the fluid is often referred to as “crystallization” or “crystallogenesis”. Protein crystals are almost always grown in solution. The most common approach is to lower the solubility of its component molecules gradually. Crystal growth in solution is characterized by two steps: nucleation of a microscopic crystallite (possibly having only 100 molecules), followed by growth of that crystallite, ideally to a diffraction-quality crystal.

Any of a variety of specialized crystallization methods for membrane proteins can be used, many of which are reviewed in Caffrey (2003 & 2009). In general terms, the methods are lipid-based methods that include adding lipid to the complex prior to crystallization. Many of these methods, including the lipidic cubic phase crystallization method and the bicelle crystallization method, exploit the spontaneous self-assembling properties of lipids and detergent as vesicles (vesicle-fusion method), discoidal micelles (bicelle method), and liquid crystals or mesophases (in meso or cubic-phase method). Lipidic cubic phases crystallization methods are described in, for example: Landau et al. 1996; Gouaux 1998; Rummel et al. 1998; Nollert et al. 2004, Rasmussen et al. 2011a and b, which publications are incorporated by reference for disclosure of those methods. Bicelle crystallization methods are described in, for example: Faham et al. 2005; Faham et al. 2002, which publications are incorporated by reference for disclosure of those methods.

“Solving the structure” as used herein refers to determining the arrangement of atoms or the atomic coordinates of a protein, and is often done by a biophysical method, such as X-ray crystallography. In many cases, obtaining a diffraction-quality crystal of a protein is the key barrier to solving its atomic-resolution structure. The herein described G protein peptidomimetics, in particular G_{q/11} protein peptidomimetics, can be used to improve the diffraction quality of the crystals so that the crystal structure of the receptor:G protein peptidomimetic complex can be solved/determined.

The term “atomic coordinates”, as used herein, refers to a position of atoms within the space of a molecular structure, typically expressed by a set of X, Y, and Z coordinates. In certain embodiments, the atomic coordinates contain additional information. A skilled person appreciates that a 3D rigid body rotation of the atomic coordinates or a translation of the atomic coordinates do not alter the structure of the described structure. An illustrative example to conduct similarity analyses is by means of software application such as the molecular similarity program QUANTA (Molecular Simulations Inc., San Diego).

In one embodiment, atomic coordinates are obtained using X-ray crystallography according to methods well-known to those of ordinarily skill in the art of biophysics. “X-ray crystallography”, as used herein, is a method of determining the arrangement of atoms within a crystal, in which a beam of X-rays strikes a crystal and diffracts into many specific directions. From the angles and intensities of these diffracted beams, a crystallographer can produce a three-dimensional picture of the density of electrons within the crystal. From this electron density, the mean positions of the atoms in the crystal can be determined, as well as their chemical bonds, their disorder and various other information. Those skilled in the art understand that a set of structure co-ordinates determined by X-ray crystallography contains standard errors. In other embodiments, atomic coordinates can be obtained using other experimental biophysical structure determination methods that can include electron diffraction (also known as electron crystallography) and nuclear magnetic resonance (NMR) methods. In yet other embodiments, atomic coordinates can be obtained using molecular modelling tools which can be based on one or more of ab initio protein folding algorithms, energy minimization, and homology-based modelling. These techniques are well known to persons of ordinary skill in the biophysical and bioinformatic arts.

### Ligand Screening and Drug Discovery

Other applications are particularly envisaged that can make use of the G protein peptidomimetics, in particular the G_{q/11} protein peptidomimetics, of the invention, including compound or fragment screening, which will be described further herein. The G protein peptidomimetics, in particular the G_{q/11} protein peptidomimetics, disclosed herein are particularly useful for the screening of compounds or fragments that selectively recognize structural features of orthosteric or allosteric sites that are unique to the active conformation of a GPCR (leading to G protein coupled signalling).

In the process of compound screening, lead optimization and drug discovery (including peptide and antibody discovery), there is a requirement for faster, more effective, less expensive and especially information-rich screening assays that provide simultaneous information on various compound characteristics and their effects on various cellular pathways (i.e. efficacy, specificity, toxicity and drug metabolism). Thus, there is a need to quickly and inexpensively screen large numbers of compounds in order to identify new specific ligands of a protein of interest, preferably conformation-selective ligands, which may be potential new drug candidates.

Alternatively, fragment-based drug discovery (FBDD) is a method to generate hits for selected drug targets. FBDD is based on the concept that the chemical space is easier filled by low molecular weight fragments than larger molecules, as used in high-throughput screenings (HTS). Fragments identified by screening techniques (functional screening, nuclear magnetic resonance, mass spectrometry and X-ray crystallography) may be optimized by elongation or combination in order to improve the affinity and reach the criteria for drug leads. In order to discover novel conformation-selective drugs, there is a need for tools that allow the identification of low-molecular weight fragments with a moderate affinity for the targeted receptors in a particular conformation such as an active conformation.

The present invention provides G protein peptidomimetics, in particular G_{q/11} protein peptidomimetics, that stabilize or lock a GPCR in a functional conformation, preferably in an active conformation. This will allow to quickly and reliably screen for and differentiate between receptor agonists, inverse agonists, antagonists and/or modulators as well as inhibitors of GPCRs, so increasing the likelihood of identifying a ligand with the desired pharmacological properties. Further, as shown herein, the G protein peptidomimetics, in particular the G_{q/11} protein peptidomimetics, described herein can be used for fragment-based screening to identify low-molecular weight fragments with a desired affinity for the active GPCR conformer. In particular, the G protein peptidomimetics, in particular the G_{q/11} protein peptidomimetics, the complexes and fusion polypeptides comprising the same, and compositions, including cellular compositions, comprising said G protein peptidomimetics, complexes or fusion polypeptides, for which specific preferences have been described herein before, are particularly suitable for this purpose, and can then be used as selection reagents for screening in a variety of contexts.

Thus, the present invention encompasses the use of the G protein peptidomimetics, in particular the G_{q/11} protein peptidomimetics described herein, complexes comprising the same, fusion polypeptides comprising the same, or compositions comprising said G protein peptidomimetics, complexes or fusion polypeptides as described hereinbefore, in screening and/or identification programs for binding partners or ligands of a GPCR, in particular a GPCR to which the G protein specifically binds. This might ultimately lead to potential new drug candidates.

In accordance, the invention method provides a (screening) method for identifying a compound capable of interacting with a GPCR, comprising:
contacting the GPCR with a test compound(s) and a G protein peptidomimetic, in particular a G_{q/11} protein peptidomimetic, fusion polypeptide, complex or composition as described herein;
evaluating binding of the test compound to the GPCR; and
optionally selecting a test compound(s) that binds to the GPCR as a compound capable of interacting with the GPCR.

In particular embodiments, the compound capable of interacting with the GPCR is a conformation-selective compound of the GPCR, in particular an active conformation-selective compound of the GPCR.

Also disclosed herein is a method of identifying conformation-selective compounds of a GPCR, the method comprising the steps of
a) providing a complex or fusion polypeptide comprising a GPCR and a G protein peptidomimetic, in particular a G_{q/11} protein peptidomimetic, capable of stabilizing the GPCR in an active conformational state, and
b) providing a test compound, and
c) evaluating whether the test compound is a conformation-selective compound for the GPCR.

Specific preferences for the G protein peptidomimetics, the G_{q/11} protein peptidomimetics, complexes, fusion polypeptides, and compositions are as defined above with respect to earlier aspects of the invention.

In embodiments, the G protein peptidomimetic, the G_{q/11} protein peptidomimetic, the GPCR or the complex or fusion polypeptide comprising the G protein peptidomimetic and the GPCR, as used in any of the screening methods described herein, are provided as whole cells, or cell (organelle) extracts such as membrane extracts or fractions thereof, or may be incorporated in lipid layers or vesicles (comprising natural and/or synthetic lipids), high-density lipoparticles, or any nanoparticle, such as nanodisks, or are provided as virus or virus-like particles (VLPs), so that sufficient functionality of the respective proteins is retained. Methods for preparations of GPCRs from membrane fragments or membrane-detergent extracts are reviewed in detail in Cooper (2004). Alternatively, the GPCR and/or the complex or fusion polypeptide may also be solubilized in detergents. High-throughput screening for binding partners or ligands of receptors may be preferred, and optionally the screening methods disclosed herein may be miniaturized in view hereof. The use of both new and known compound libraries is envisaged in the present invention. Also envisaged herein is the use of low-molecular weight fragment libraries. The size of the compound or fragment library is not limiting.

This may be facilitated by immobilization of either the G protein peptidomimetic, the G_{q/11} protein peptidomimetic, the complex or the fusion polypeptide as described herein onto a suitable solid surface or support that can be arrayed or otherwise multiplexed. Accordingly, in embodiments, the G protein peptidomimetic, the G_{q/11} protein peptidomimetic, the complex or the fusion polypeptide are immobilized to a solid support.

Non-limiting examples of suitable solid supports include beads, columns, slides, chips or plates. More particularly, the solid supports may be particulate (e. g. beads or granules, generally used in extraction columns) or in sheet form (e. g. membranes or filters, glass or plastic slides, microtiter assay plates, dipstick, capillary fill devices or such like) which can be flat, pleated, or hollow fibres or tubes. The following matrices are given as examples and are not exhaustive, such examples could include silica (porous amorphous silica), e.g. the FLASH series of cartridges containing 60A irregular silica (32-63 um or 35-70 um) supplied by Biotage (a division of Dyax Corp.); agarose or polyacrylamide supports, for example the Sepharose range of products supplied by Amersham Pharmacia Biotech, or the Affi-Gel supports supplied by Bio-Rad. In addition, there are macroporous polymers, such as the pressure-stable Affi-Prep supports as supplied by Bio-Rad. Other supports that could be used include, without limitation, dextran, collagen, polystyrene, methacrylate, calcium alginate, controlled pore glass, aluminium, titanium and porous ceramics. Alternatively, the solid surface may comprise part of a mass dependent sensor, for example, a surface plasmon resonance detector. Further examples of commercially available supports are discussed in, for example, Protein Immobilization, R. F. Taylor ed., Marcel Dekker, Inc., New York, (1991). Immobilization may be either non-covalent or covalent. In particular, non-covalent immobilization or adsorption on a solid surface of the G protein peptidomimetic, or the complex or the fusion polypeptide comprising the G protein peptidomimetic and the GPCR, may occur via a surface coating with any of an antibody, or streptavidin or avidin, or a metal ion, recognizing a molecular tag attached to the G protein peptidomimetic, according to standard techniques known by the skilled person (e.g. biotin tag, histidine tag, etc.). Alternatively, G protein peptidomimetic, or the complex or fusion polypeptide comprising the G protein peptidomimetic and the GPCR, may be attached to a solid surface by covalent cross-linking using conventional coupling chemistries. A solid surface may naturally comprise cross-linkable residues suitable for covalent attachment or it may be coated or derivatized to introduce suitable cross-linkable groups according to methods well known in the art. Sufficient functionality of the immobilized protein can be retained following direct covalent coupling to the desired matrix via a reactive moiety that does not contain a chemical spacer arm. Advances in molecular biology, particularly through site-directed mutagenesis, enable the mutation of specific amino acid residues in a protein sequence. The mutation of a particular amino acid (in a protein with known or inferred structure) to a lysine or cysteine (or other desired amino acid) can provide a specific site for covalent coupling, for example. It is also possible to reengineer a specific protein to alter the distribution of surface available amino acids involved in the chemical coupling (Kallwass et al, 1993), in effect controlling the orientation of the coupled protein. A similar approach can be applied to the G protein peptidomimetics, thereby minimizing disruption to the GPCR-binding activity of the G protein peptidomimetic, so providing a means of oriented immobilization without the addition of other peptide tails or domains containing either natural or unnatural amino acids.

Conveniently, the immobilized proteins described herein may be used in immunoadsorption processes such as immunoassays, for example ELISA, or immunoaffinity purification processes by contacting the immobilized proteins with a test sample according to standard methods conventional in the art. Alternatively, and particularly for high-throughput purposes, the immobilized proteins can be arrayed or otherwise multiplexed.

In other embodiments, the test compound (or a library of test compounds) may be immobilized on a solid surface, such as a chip surface, whereas the G protein peptidomimetic and GPCR, the complex or the fusion polypeptide as described herein are provided, for example, in a detergent solution or in a membrane-like preparation or composition.

In yet other embodiments, neither the G protein peptidomimetic, nor the GPCR, nor the test compound is immobilized, for example in phage-display selection protocols in solution, or radioligand binding assays. For example, the GPCR, as used in any of the screening methods described herein, may be provided as whole cells, or cell (organelle) extracts such as membrane extracts or fractions thereof, wherein the GPCR is embedded in the cell wall or cell membrane fragment, or the GPCR may be incorporated in lipid layers or vesicles (comprising natural and/or synthetic lipids), high-density lipoparticles, or any nanoparticles, such as nanodisks, or as virus or virus-like particles (VLPs) as described above. Typically, the G protein peptidomimetic and its binding epitope (which typically comprises amino acid residues from the intracellular loops of the GPCR) are on one side (which may be referred to as the “intracellular” side) of respectively, the cell wall, the cell membrane, the lipid layer or vesicle, lipoparticle, nanoparticle, etc. whereas the test compound(s) is on the other side (which may be referred to as the “extracellular” side).

Screening assays for drug discovery can be solid phase (e.g. beads, columns, slides, chips or plates) or solution phase assays, e.g. a binding assay, such as radioligand binding assays.

In high-throughput assays, it is possible to screen up to several thousand different compounds or low-molecular weight fragments, in a single day in 96-, 384- or 1536-well formats. For example, each well of a microtiter plate can be used to run a separate assay against a selected test compound, or, if concentration or incubation time effects are to be observed, every 5-10 wells can test a single test compound. Thus, a single standard microtiter plate can assay about 96 test compounds. It is possible to assay many plates per day; assay screens for up to about 6.000, 20.000, 50.000 or more different compounds are possible today.

Various methods may be used to determine binding between the (active conformation stabilized) GPCR and a test compound, including for example, flow cytometry, radioligand binding assays, enzyme linked immunosorbent assays (ELISA), surface plasmon resonance assays, chip-based assays, immunocytofluorescence, yeast two-hybrid technology and phage display which are common practice in the art, for example, in Sambrook et al. (2001), Molecular Cloning, A Laboratory Manual. Third Edition. Cold Spring Harbor Laboratory Press, Cold Spring Harbor, NY. Other methods of detecting binding between a test compound and a GPCR include ultrafiltration with ion spray mass spectroscopy/HPLC methods or other (bio)physical and analytical methods. Fluorescence Energy Resonance Transfer (FRET) methods, for example, well known to those skilled in the art, may also be used. It will be appreciated that a bound test compound can be detected using a unique label or tag associated with the compound, such as a peptide label, a nucleic acid label, a chemical label, a fluorescent label, or a radioactive isotope label, as described further herein.

The test compound may thus optionally be covalently or non-covalently linked to a detectable label. Suitable detectable labels and techniques for attaching, using and detecting them will be clear to the skilled person. Non-limiting examples include detection by spectroscopic, photochemical, biochemical, immunochemical, electrical, optical or chemical means. Useful labels include magnetic beads (e.g. dynabeads), fluorescent dyes (e.g. all Alexa Fluor dyes, fluorescein isothiocyanate, Texas red, rhodamine, green fluorescent protein and the like), radiolabels (e.g. ³H, ¹²⁵I, ³⁵S, ¹⁴C, or ³²P), enzymes (e.g. horse radish peroxidase, alkaline phosphatase), and colorimetric labels such as colloidal gold or coloured glass or plastic (e.g. polystyrene, polypropylene, latex, etc.) beads. Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the coloured label.

The compounds to be tested can be any small chemical compound, a macromolecule (such as a protein, a sugar, nucleic acid or lipid), as well as a low-molecular weight fragment. In embodiments, the test compound used in any of the screening methods described herein is selected from the group comprising a polypeptide, a peptide, a small molecule, a natural product, a peptidomimetic, a nucleic acid, a lipid, a lipopeptide, a carbohydrate, an antibody or any fragment derived thereof, such as Fab, Fab′ and F(ab′)2, Fd, single-chain Fvs (scFv), single-chain antibodies, disulfide-linked Fvs (dsFv) and fragments comprising either a VL or VH domain, a heavy chain antibody (hcAb), a single domain antibody (sdAb), a minibody, the variable domain derived from camelid heavy chain antibodies (VHH or Nanobody), the variable domain of the new antigen receptors derived from shark antibodies (VNAR), a protein scaffold including an alphabody, protein A, protein G, designed ankyrin-repeat domains (DARPins), fibronectin type III repeats, anticalins, knottins, engineered and CH2 domains (nanoantibodies). For example, test compounds may be small chemical compounds, peptides, antibodies, or (low-molecular weight) fragments thereof.

It will be appreciated that in some embodiments the test compound may be a library of test compounds. For example, high-throughput screening assays for therapeutic compounds such as agonists, antagonists or inverse agonists and/or modulators are envisaged herein. For high-throughput purposes, compound libraries or combinatorial libraries may be used such as allosteric compound libraries, peptide libraries, antibody libraries, fragment-based libraries, synthetic compound libraries, natural compound libraries, phage-display libraries and the like. Methodologies for preparing and screening such libraries are known to those of skill in the art. For example, high-throughput screening methods may involve providing a combinatorial chemical or peptide library containing a large number of potential therapeutic ligands. Such “combinatorial libraries” or “compound libraries” are then screened in one or more assays, as described herein, to identify those library members (particular chemical species or subclasses) that display a desired characteristic activity. A “compound library” as used herein refers to a collection of stored chemicals usually used ultimately in high-throughput screening A “combinatorial library” refers to a collection of diverse chemical compounds generated by either chemical synthesis or biological synthesis, by combining a number of chemical “building blocks” such as reagents. Preparation and screening of combinatorial libraries are well known to those of skill in the art.

The compounds thus identified can serve as conventional “lead compounds” or can themselves be used as potential or actual therapeutics. Thus, in one further embodiment, the screening methods as described herein further comprises a step of modifying a test compound which has been shown to selectively bind to a GPCR in a particular conformation, in particular an active conformation, and determining whether the modified test compound binds to the GPCR when residing in the particular conformation.

In embodiments, it is determined whether the test compound alters the binding of a receptor ligand (as defined herein) to the GPCR. Preferably, the receptor ligand is chosen from the group comprising a small molecule, a polypeptide, an antibody or any fragment derived thereof, a natural product, and the like. More preferably, the receptor ligand is a full agonist, or a partial agonist, a biased agonist, an antagonist, or an inverse agonist, as described hereinbefore. Binding of a ligand to this receptor can be assayed using standard ligand binding methods known in the art as described elsewhere herein. For example, a ligand may be radiolabelled or fluorescently labelled. The compound will be characterized by its ability to alter the binding of the labelled ligand. The compound may decrease the binding between the ligand and the receptor, or may increase the binding between the ligand and the receptor, for example by a factor of at least 2 fold, 3 fold, 4 fold, 5 fold, 10 fold, 20 fold, 30 fold, 50 fold, 100 fold.

In embodiments, the test compound as used in any of the herein described screening methods is provided as a biological sample. In particular, the sample can be any suitable sample taken from an individual. For example, the sample may be a body fluid sample such as blood, serum, plasma, spinal fluid.

In addition to establishing binding to a GPCR in a particular conformation of interest, it will also be desirable to determine the functional effect of a compound on the receptor. For example, the compounds may bind to the GPCR resulting in the modulation (activation or inhibition) of the biological function of the receptor, in particular the downstream receptor signalling. This modulation of intracellular signalling can occur ortho- or allosterically. The compounds may bind to the GPCR so as to activate or increase receptor signalling; or alternatively so as to decrease or inhibit receptor signalling. The compounds may also bind to the GPCR in such a way that they block off the constitutive activity of the receptor. The compounds may also bind to the GPCR in such a way that they mediate allosteric modulation (e.g. bind to the receptor at an allosteric site). In this way, the compounds may modulate the receptor function by binding to different regions in the receptor (e.g. at allosteric sites). Reference is for example made to George et al. 2002; Kenakin 2002; Rios et al. 2001. The compounds may also bind to the GPCR in such a way that they prolong the duration of the receptor-mediated signalling or that they enhance receptor signalling by increasing receptor-ligand affinity. Further, the compounds may also bind to the GPCR in such a way that they inhibit or enhance the assembly of receptor functional homomers or heteromers. The efficacy of the compounds and/or compositions comprising the same, can be tested using any suitable in vitro assay, cell-based assay, in vivo assay and/or animal model known per se, or any combination thereof, depending on the specific disease or disorder involved.

It will be appreciated that the G protein peptidomimetics, in particular the G_{q/11} protein peptidomimetics, complexes, fusion polypeptides, and compositions comprising the same as described herein, may be further engineered and are thus particularly useful tools for the development or improvement of cell-based assays. Cell-based assays are critical for assessing the mechanism of action of new biological targets and biological activity of chemical compounds. For example, without the purpose of being limitative, current cell-based assays for GPCRs include measures of pathway activation (Ca²⁺ release, cAMP generation or transcriptional activity); measurements of protein trafficking by tagging GPCRs and downstream elements with GFP; and direct measures of interactions between proteins using Fórster resonance energy transfer (FRET), bioluminescence resonance energy transfer (BRET) or yeast two-hybrid approaches.

In embodiments, the complex or fusion polypeptide described herein comprising the GPCR and the G protein peptidomimetic, in particular the G_{q/11} protein peptidomimetic, that specifically binds to the GPCR may be used for the selection of binding agents including antibodies or antibody fragments that bind the receptor by any of the screening methods as described above. Persons of ordinary skill in the art will recognize that such binding agents, as a non-limiting example, can be selected by screening a set, collection or library of cells that express binding agents on their surface, or bacteriophages that display a fusion of genIII and binding agent at their surface, or yeast cells that display a fusion of the mating factor protein Aga2p, or by ribosome display amongst others.

### Allosteric Modulator

A further aspect relates to use, preferably in vitro use, of the G protein peptidomimetic, in particular the G_{q/11} protein peptidomimetic, as an allosteric modulator of a GPCR, preferably a G_{q/11} protein-coupled receptor.

An “allosteric modulator” generally refers to a substance that binds to a receptor at a site which is not the orthosteric binding site of an endogenous ligand (e.g. an agonist), and which is able to influence the affinity and/or efficacy of the orthosteric ligand for the receptor. Allosteric modulators include positive allosteric modulators (PAMs) and negative allosteric modulators (NAMs).

The binding of a G protein peptidomimetic to an allosteric site of a GPCR may result in conformational changes which influence or modulate, e.g. (allosterically) potentiate or (allosterically) suppress or attenuate, GPCR signalling or the response of the GPCR to binding by an orthosteric binding site ligand such as an agonist.

In certain embodiments, the G protein peptidomimetic is used as an intracellular allosteric modulator of a GPCR. Preferably, the G protein peptidomimetic is modified to comprise a cell-penetrating peptide (CPP) to be used as intracellular allosteric modulator of a GPCR.

### Biosensor

Yet a further aspect relates to use, preferably in vitro use, of the G protein peptidomimetic, in particular the G_{q/11} protein peptidomimetic, as a biosensor e.g. as a biosensor for a conformational change of a GPCR (e.g. to detect a substance or compound that binds to a GPCR and alters the conformation of the GPCR), as a biosensor for assessing the localization and/or trafficking of a GPCR, and/or as a biosensor for investigating a GPCR signalling pathway.

A change in GPCR conformation, e.g. resulting form the binding of a ligand or an allosteric modulator, may be detected by a change in the binding of the conformation-sensitive G protein peptidomimetic or G_{q/11} protein peptidomimetic to the GPCR. The method not only allows to identify ligands of the GPCR that directly modulate the biological activity of the GPCR, but any substance that change the GPCR conformation and that may modulate the biological activity of the GPCR in a subtle manner (e.g. allosteric modulators).

Preferably, the G protein peptidomimetic is modified to comprise a fluorescent probe or label to be used biosensor.

### Kit of Parts

Still another aspect of the invention relates to a kit comprising a G protein peptidomimetic, in particular a G_{q/11} protein peptidomimetic, capable of stabilizing a GPCR in an active conformational state, optionally as a fusion polypeptide with the GPCR, or a kit comprising a composition as described herein comprising such G protein peptidomimetic or fusion polypeptide. In further examples, the kit of parts may comprise a cellular expression system comprising an oligonucleotide sequence encoding the G protein peptidomimetic as described herein, optionally as a fusion polypeptide with the GPCR. In certain embodiments, the G protein peptidomimetic is encoded in the genome of the cellular expression system. The kit may further comprise a combination of reagents such as buffers, molecular tags, vector constructs, reference sample material, as well as a suitable solid supports, and the like. Such a kit may be useful for any of the applications of the present invention as described herein. For example, the kit may further comprise (a library of) test compounds useful for compound screening applications.

The present invention will now be further illustrated by means of the following non-limiting examples.

### EXAMPLES

### Material and Methods

### General

Analytical high-performance liquid chromatography (HPLC) analysis was performed on a Hitachi Chromaster system (Chromaster HPLC 5260 autosampler, Chromaster HPLC 5160 Pump, Chromaster HPLC 5310 column and a Chromaster HPLC 5430 diode array detector). The mobile phase consisted of 0.1% trifluoroacetic acid (TFA) in acetonitrile (AcN) and 0.1% TFA in Milli-Q water. The analyzed peptides eluted through a column with a gradient from 1% to 100% of AcN over 5 min at a flow rate of 3 ml/min.

For liquid chromatography-mass spectrometry (LC-MS), a Micromass Q-Tof Micro system, attached to a Waters 600 analytical HPLC system with an autosampler, a Waters 2696 pump and a Grace Vydac C18 column (25 cm×4.6 mm×5 μm) was used to determine the masses present in the (peptide) samples. Products were detected by a Waters 2489 UV/visible detector at a wavelength of 215 nm. Data collection and spectrum analysis was done with Masslynx software. The solvents used to run a LC-MS were similar to those of the HPLC except that TFA was replaced by formic acid. LC-MS samples were prepared in the same manner as for the analysis with the analytical HPLC. The used gradient ran from 3% to 97% AcN in 20 min at a flow rate of 0.3 ml/min.

Preparative RP-HPLC purification of crude peptide products was performed on a Gilson HPLC system accommodated with Gilson 322 pumps over a Supelco Discovery® BIO Wide Pore C18 column (25 cm×21.2 mm, 10 μm) using a UV/Vis-156 detector at 215 nm and controlled by the software package Unipoint. An identical solvent system as for the analytical HPLC was used but with a flow rate of 20 ml/min. Prior injection, the crude peptide product solution was filtered using a CHROMAFIL® syringe filter. The collected fractions were lyophilized on the Virtis BenchTop Pro with Omnitronics™ (3I) to remove the water and AcN and retrieve the purified peptide as a white powder.

The mass of the purified peptides was controlled by high resolution mass spectroscopy (HRMS) on a Micromass Q-Tof Micro system equipped with an electrospray ionization.

### Peptide Synthesis

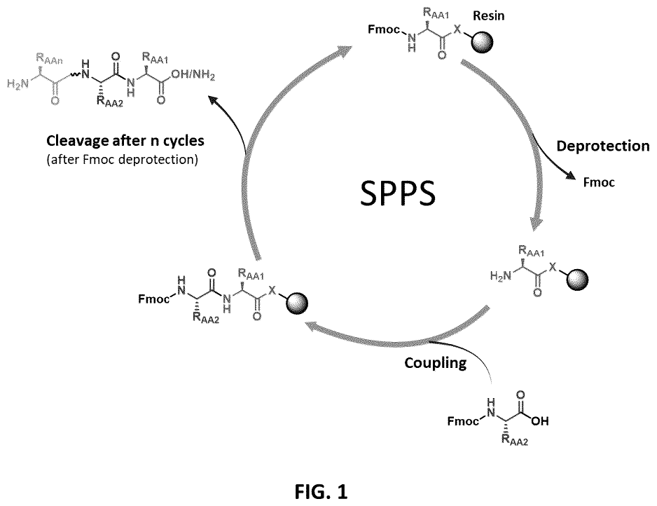

All peptides were synthesized using fluorenylmethyloxycarbonyl (Fmoc)-based solid phase peptide synthesis (SPPS) on an automated synthesizer and/or manually, depending on the sequence (FIG. 1). The synthesis was performed on preloaded Fmoc-Leu-Wang resin (loading 0.6-0.75 mmol/g) or Rink Amide resin (loading 0.92 mmol/g) depending on the desired C-terminal end of the peptide, being a carboxylic acid or carboxamide. The resin was first swollen during 20 min in dichloromethane (DCM) followed by the Fmoc deprotection twice using a solution of 20% 4-methylpiperidine in DMF, for 5 min and 15 min, respectively. Then the resin was washed with N′,N′-dimethylformamide (DMF) and DCM. During the manual synthesis 3 equiv. Of Fmoc-protected amino acid (1.5 equiv. For unnatural amino acids) was added to the coupling mixture, consisting of 3 equiv. Of o-(benzotriazol-1-yl)-N,N,N′,N′-tetramethyluronium hexafluorophosphate (HBTU) and 4 equiv. Of N,N-diisopropylethylamine (DIPEA) in DMF and let shaking for 40 min (1.5 h when 1.5 equiv. Is used). After each coupling, the mixture was filtered off and the resin was washed with DMF and DCM. Before every coupling, the resin was treated with 20% 4-methylpiperidine for Fmoc-deprotection.

Using the automatic synthesizer (Activo-P11 or CEM Liberty Blue™), the coupling was performed with 5 equiv. Of Fmoc-protected amino acid (2 equiv. For unnatural amino acids) in a solution of 0.5 M HBTU and 1 M DIPEA in DMF for the automated Activo-P11 synthesizer and 0.5 M N,N′-diisopropylcarbodiimide (DIC) and 1 M Oxyma in DMF for the CEM Liberty Blue™. At the end of the synthesis the resin was removed from the synthesizer and washed several times with DCM. The acetylation of the N-terminus was performed manually with 10 equiv. Of acetic anhydride and 5 equiv. Of DIPEA during 1 h in DMF at room temperature. The peptides with free N-terminus, were tert-butyloxycarbonyl (Boc)-protected with 4 equiv. Of Boc-ON and 5 equiv. Of DIPEA in DMF during 2 h, before cyclization.

After completion of the peptides, they were cleaved from the resin using a cocktail solution consisting of 95% TFA, 2.5% triisopropylsilane and 2.5% distilled water during 4 h. Finally, the crude peptides were obtained after freeze-drying. The purification of the crude products was performed using a preparative HPLC to obtain the peptide (TFA salt) as a powder with a high purity (>97%).

### Peptide Cyclization

Cyclization via Cu(I)-catalyzed azide-alkyne cycloaddition was performed using 24 equiv. Of CuBr and 24 equiv. DIPEA in DMF, during 7 h. The copper was removed by washing the resin with a solution of 1 M pyridine hydrochloride in DCM/MeOH (95:5), followed by washing steps with DMF and DCM.

### Synthesis of Peptides SBL-GQ-13-25 of Example 5

All peptides were synthesized using Fmoc-based solid phase peptide synthesis (SPPS) on an automated synthesizer and/or manually, depending on the sequence. The synthesis was performed on 2-chlorotrityl chloride resin (loading 0.8 mmol/g) or on preloaded Fmoc-Leu-Wang resin (loading 0.6-0.75 mmol/g), depending on the method of fluorophore coupling. For chlorotrityl, the resin was first swollen in DCM during 20 min followed by anchorage of the first Fmoc-protected amino acid (2 equiv.) to the resin in presence of DIPEA (2 equiv.) in DMF during 2 h, followed by capping with a mixture of DCM/MeOH/DIPEA (8.5:1:0.5). For Fmoc-Leu-Wang, the resin was first swollen during 20 min in DCM followed by the Fmoc deprotection twice using a solution of 20% 4-methylpiperidine in DMF, for 5 min and 15 min, respectively. Then the resin was washed with DMF and DCM. During the manual synthesis 3 equiv. of Fmoc-protected amino acid (1.5 equiv. for unnatural amino acids) was added to the coupling mixture, consisting of 3 equiv. of HBTU and 4 equiv. of DIPEA in DMF and let shaking for 40 min (1.5 h when 1.5 equiv. is used). For difficult coupling reactions, such as for the repeated arginine and tryptophan residues in the cell-penetrating peptide motif (CPP), the coupling was performed twice with a fresh coupling mixture, during 1 h. After each coupling, the mixture was filtered off and the resin was washed with DMF and DCM. Before every coupling, the resin was treated with 20% 4-methylpiperidine for Fmoc-deprotection.

Using the automatic synthesizer (Activo-P11 or CEM Liberty Blue™), the coupling was performed with 5 equiv. of Fmoc-protected amino acid (2 equiv. for unnatural amino acids) in a solution of 0.5 M HBTU and 1 M DIPEA in DMF for the automated Activo-P11 synthesizer and 0.5 M DIC and 1 M Oxyma in DMF for the CEM Liberty Blue™. Difficult coupling reactions were performed twice, using the same conditions. At the end of the synthesis the resin was removed from the synthesizer and washed several times with DCM.

Cyclization was performed via a Cu(I)-catalyzed azide-alkyne cycloaddition, using 24 equiv. of CuBr and 24 equiv. DIPEA in DMF, during 7 h. The copper was removed by washing the resin with a solution of 1 M pyridine hydrochloride in DCM/MeOH (95:5), followed by washing steps with DMF and DCM.

Peptide SBL-GQ-16, -17, -18, -19 and -20 were synthesized on 2-chlorotrityl chloride resin, to allow cleavage of the peptide from the resin without removal of the side chain protecting groups. Therefore, after completion of the peptide (without fluorophore), HFIP/DCM (1:4) was added to the resin and let shaking for 2 h. After evaporation of HFIP/DCM and freeze-drying, the N-terminal free, side chain protected peptides were incubated overnight with Pacific Blue NHS ester (SBL-GQ-16) (1.2 equiv.), DY647-P1 NHS ester (SBL-GQ-17) (1.1 equiv.) or Sulfocyanine 3 NHS ester (SBL-GQ-18, -19 and -20) (0.8 equiv.) and DIPEA (10 equiv.), in the dark. After completion of the reaction, the peptides were fully deprotected using a cocktail solution consisting of 95% TFA, 2.5% triisopropylsilane and 2.5% distilled water during 3-7 h, depending on the number of residues in the cell-penetrating peptide motif and the acid-sensitivity of the fluorophore. The purification of the crude products was performed using a preparative HPLC to obtain the peptide (TFA salt) as a powder with a high purity (>97%).

Peptides SBL-GQ-21, -22, -23 and -24 were synthesized on Fmoc-Leu-Wang resin. After completion of the peptides (until N-terminal cysteine), they were cleaved from the resin using a cocktail solution consisting of 95% TFA, 2.5% triisopropylsilane and 2.5% distilled water, during 3-7 h. The crude peptides were obtained after freeze-drying and purified using a preparative HPLC. Next, the reaction of Sulfocyanine 5 maleimide (1 equiv.) to the side chain of the N-terminal cysteine residue was performed in the dark, in 10 mM Tris buffer (pH 6.8), under Argon. A final purification was performed to obtain the peptide (TFA salt) as a powder with a high purity (>97%).

### Receptor Membrane Extracts Preparation

The membrane extracts were prepared from cells that overexpressed muscarinic acetylcholine 1 receptor (M1R), by resuspending the cell pellet in a buffer (1 ml buffer/2×1E7 cells) containing 20 mM Hepes (pH 7.4), 100 mM NaCl, Leupeptin and phenylmethylsulfonyl fluoride (PMSF). Afterwards the cells were vortexed and homogenized in ice using a small volume ULTRA-TURRAX® (6×10 sec). The cells were then centrifuged for 15 min at 16000 rcf, in a pre-cooled centrifuge (4° C.) and resuspended in the previous buffer containing 10% sucrose. Finally, the membranes were again homogenized in ice with the small volume ULTRA-TURRAX® (3×10 sec). The protein concentration in the membrane extracts was determined using a Pierce™ bicinchoninic acidv (BCA) Protein Assay Kit and the protein concentration was extrapolated from the Bovine Serum Albumine standard curve.

### Radioligand Binding Assay

The M1R constructs used in this assay consisted of the full-length human muscarinic 1 receptor with a FLAG tag at the N-terminus. A buffer containing 20 mM Hepes pH 7.4, 100 mM NaCl, and 0.1% BSA was used for the ligands, membrane extracts and peptides (+1% dimethylsulfoxide (DMSO)). The competition assay was performed with [3H]—N-methyl scopolamine ([3H]-NMS) as radiolabeled antagonist, at a final concentration of 0.6 nM. A ten-fold dilution series of the agonist, acetylcholine chloride, was prepared to obtain a dose response curve. After incubation, the samples were harvested into filter plates (GF/C) and washed with ice cold washing buffer (20 mM Hepes pH 7.4) using the 96-well harvester. After drying in the oven during 1 h and the addition of scintillation liquid, the plates were placed in the MICROBETA® scintillation counter to measure the remaining radioactivity.

The competition curves were generated using Graphpad Prism 6.0. The raw data were normalized and a nonlinear regression was used to fit the data in a one-site binding model, with the total binding set as 100% and the non-specific binding between 0 and 5%.

### Bimane Fluorescence Assay

The bimane fluorescence assays on the ghrelin receptor were performed as described in Damian et al. (2021. Nat. Commun. 12:1-15). Briefly, E. coli bacteria (BL21(DE3)) were transformed with a vector encoding the human ghrelin receptor with an integrin α₅ fragment at the N-terminus and a polyhistidine tag at the C-terminus. The receptors were then purified and reconstituted into lipidated nanodiscs. The monobromobimane labeling was performed by incubating the receptors, with a unique reactive cysteine at position 255, during 16 h in the dark at 4° C. and in the presence of 0.1 mM tris(2-carboxyethyl)phosphine (TCEO). The reaction was terminated with 5 mM L-cysteine and unreacted monobromobimane was removed using a Zeba™ Spin desalting column.

For the fluorescence assay, the labeled receptor was incubated during 2 h at 20° C. (0.2 μM final concentration), in the absence or presence of the full agonist JMV1843 (20 μM) and in the absence or presence of either the peptidomimetics at varying molar ratios or the purified Gα_{q}β₁γ₂ heterotrimer, at a 1:5 receptor-to-G protein molar ratio. Afterwards, the fluorescence experiments were performed on a Horiba Fluoromax-4 TCSP spectrofluorimeter. For each scan, the excitation wavelength (λ_{exc}) was set at 380 nm and emission was collected between 440 nm and 520 nm.

### IP-One G_{q} Assay

IP-One assays were conducted by using a homogeneous time-resolved fluorescence resonance energy transfer (TRFRET) assay (cisbio, IP-One G_{q} kit). The assays were performed on 384-well plates, containing 5000 cells/well. The HEK293 cells, in stimulation buffer (1×), were incubated with 5 or 10 μM of peptides (in stimulation buffer) during 1 h at 37° C. Next, the GHSR agonist MK0677 was added at the desired concentration (in stimulation buffer), and incubated for 45 min at 37° C. Finally, the d2-labeled IP1 and anti-IP1-cryptate, diluted in lysis buffer, were added to each well and incubated at room temperature. After 2 h of incubation in the dark, the plates were analyzed using the PHERAstar microplate reader.

### Cytotoxicity Assays

HEK293 1C8 cells were seeded 1 day prior the assay at 40,000 cells/well (or 2 days prior at 20,000 cells/well) into 96-well plates. After overnight incubation (37° C., 5% CO₂), cells were treated with different concentrations of peptides, for 3 h 45 min. Cells were washed two times with PBS and incubated with 3-(4,5-dimethylthiazol-2-yl)-2,5-diphenyltetrazolium bromide (MTT) during 3 h, at 37° C. Afterwards, cells were washed once with PBS and then incubated for 10 min with DMSO. Plates were analysed with a Tecan Spark 10 M microplate reader.

### Cell Internalization Assay

HEK293 1C8 cells were seeded 1 day prior the assay at 40,000 cells/well (or 2 days prior at 20,000 cells/well) into black 96-well plates. After overnight incubation (37° C., 5% CO2), cells were treated with 5 and 10 μM of the fluorescently labeled peptides, for 3 h 45. Cells were then washed four times with PBS and analysed using a spectrofluorometer (FluoroMax-4)

### Fluorescence Microscopy

To visualise cell-penetration of the peptides, HEK293 1C8 cells were seeded in 12-well plates, covered with a coverslip, at 120,000 cells/well. After overnight incubation (37° C., 5% CO2), cells were washed with PBS and treated with the fluorescently labeled peptides for 2 h45. Next, BG-fluorescein was added to the mixture, for Snap tag labeling of the cell-surface GHSR. After incubation of 1 h the cells were washed four times with PBS. Cells were then fixed using 4% paraformaldehyde in PBS for 5 min and washed two times with PBS. Finally, Hoechst was diluted 1/1000 in PBS, added to the fixed cells during 10 min, and washed thrice with PBS.

The cells for permeabilization were first incubated with BG-fluorescein during 1 h, followed by four times washing with PBS. The cells were then fixed with 4% paraformaldehyde in PBS for 5 min washed twice. Afterwards, the cells were permeabilized with Triton 0.1% in PBS during 5 min and washed thrice. Finally, the cells were incubated for 3 h 45 with the peptide and washed three times before Hoechst was added for 10 min.

### Statistics

Statistical analyses were all carried out with GraphPad Prism 6.

### Example 1: Design and Synthesis of Gα_{q/11} Peptidomimetics

Based on the different cryo-EM structures of the GPCRs coupled to G_{q/11} or mini-G_{q}, and similarly to GS-coupled receptors, an outward movement of TM5 and TM6 is observed upon ligand binding and receptor activation. These movements create a cavity for the engagement of the α₅ helix from the G protein, which is part of the interaction domains between the receptor and the G protein. One of the most distinct features, when analyzing the cryo-EM of the M1R-G₁₁ complex, is the pronounced intracellular TM5 extension upon receptor activation (Protein Data Bank (PDB) ID: 6OIJ).

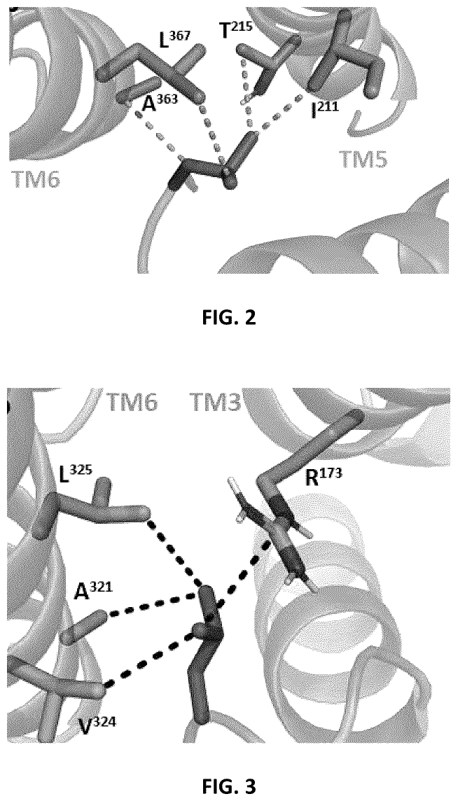

In contrast to the equivalent regions in the Gₛ and G_{i/o} which are rather negatively charged, the TM5 extension interacts with G₁₁ through hydrophobic interactions and a salt bridge between D346 of the as helix and R218 of M1R as defined by SEQ ID NO: 15 (FIG. 2). Based on mutagenesis studies, five residues located in the TM5 and TM6 of MIR were identified to be crucial for G_{q/11} coupling, two of which are interacting with the α₅ helix (F³⁴¹AAVKDTILQLNLKEYNLV³⁵⁹, SEQ ID NO:13), namely A363 and L367 that form Van der Waals interactions (VDW) with the highly conserved L358 of the G protein (FIG. 2 and Table 1). The other critical residues interact within the TM5 and TM6. Without wishing to be bound by any theory, these interactions may provide conformational stabilization.

TABLE 1
Interacting residues of the α₅ helix from Gα₁₁
with M1R as defined by SEQ ID NO: 15.

| Residue | Residue | | | | | |
|---|---|---|---|---|---|---|
| of | of | | Inter- | | between | |
| Gα₁₁^{[1]} | M1R^{[1]} | BWN^{[2]} | action | d (Å) | Gα₁₁ | receptor |
| F³⁴¹ | L¹³¹ | L^{34.51} | VDW | 3.4 | m-Ar | δ |
| A³⁴² | L²²⁵ | L^{5.75} | VDW | 3.8 | β | δ |
| A³⁴³ | / | / | | | | |
| V³⁴⁴ | L¹³¹ | L^{34.51} | VDW | 4.5 | γ | δ |
| K³⁴⁵ | L¹³¹ | L^{34.51} | VDW | 3.7 | ε | δ |
| | P¹³⁰ | P^{34.50} | VDW | 3.8 | γ | γ |
| D³⁴⁶ | L²²⁵ | L^{5.75} | VDW | 3.6 | β | γ |
| | R²¹⁸ | R^{5.68} | HB x2 | 1.8-1.8 | COO⁻ | Guanidyl |
| T³⁴⁷ | / | / | | | | |
| I³⁴⁸ | P¹³⁰ | P^{34.50} | VDW | 3.9 | γ | β |
| | L¹³¹ | L^{34.51} | VDW | 3.8 | δ | δ |
| | R¹³⁴ | R^{34.54} | VDW | 4.4 | γ | δ |
| L³⁴⁹ | V¹²⁷ | V^{3.54} | VDW | 4.1 | δ | γ |
| | P¹³⁰ | P^{34.50} | VDW | 4.5 | β | γ |
| | R²¹⁸ | R^{5.68} | VDW | 3.9 | δ | γ |
| Q³⁵⁰ | R²¹⁸ | R^{5.68} | HB | 1.9 | CO | Guanidyl |
| L³⁵¹ | / | / | | | | |
| N³⁵² | S¹²⁶ | S^{3.53} | HB | 1.9 | NH₂ | CO^{BB} |
| | N⁶⁰ | N^{2.39} | VDW | 4.3 | CO | NH₂ |
| L³⁵³ | V¹²⁷ | V^{3.54} | VDW | 3.8 | δ | γ |
| | T²¹⁵ | T^{5.65} | VDW | 3.5 | δ | OH |
| | R²¹⁸ | R^{5.68} | VDW | 3.2 | δ | Guanidyl |
| K³⁵⁴ | K⁴²³ | K^{8.48} | VDW | 4.8 | NH₃⁺ | δ |
| F³⁵⁵ | N⁶⁰ | N^{2.39} | HB | 1.7 | COO⁻ | NH₂ |
| | N⁴²² | N^{8.47} | HB | 2.4 | COO⁻ | NH₂ |
| Y³⁵⁶ | N⁶⁰ | N^{2.39} | VDW | 3.9 | OH | NH₂ |
| | D¹²² | D^{3.49} | VDW | 3.8 | OH | γ |
| | R¹²³ | R^{3.50} | ±p | 3.9 | o-Ar | γ |
| | | | (Splayed) | | | |
| | S¹²⁶ | S^{3.53} | HB | 1.8 | OH | OH |
| | C⁴²¹ | C^{7.56} | HB | 3.8 | CO^{BB} | S |
| N³⁵⁷ | K³⁶² | K^{6.32} | HB | 1.8 | CO | NH₃⁺ |
| | C⁴²¹ | C^{7.56} | HB | 3.6 | NH₂ | S |
| | | | (weak) | | | |
| | R⁴²⁶ | R^{8.51} | VDW | 4.2 | CO | Guanidyl |
| L³⁵⁸ | I²¹¹ | I^{5.61} | VDW | 3.5 | δ | γ |
| | T²¹⁵ | T^{5.65} | VDW | 4.4 | δ | γ |
| | A³⁶³ | A^{6.33} | VDW | 3.7 | β | α |
| | L³⁶⁷ | L^{6.37} | VDW | 4.1 | δ | δ |
| V³⁵⁹ | R²¹⁸ | R^{5.68} | VDW | 4.4 | δ | Guanidyl |
| | K³⁶¹ | K^{6.31} | VDW | 4.9 | CO^{BB} | NH₃⁺ |

^{[1]}Residue numbering based on cryo-EM structure (PDB: 6OIJ).
^{[2]}Residue numbering according to the Ballesteros-Weinstein numbering from the sequence alignments in the G protein-coupled receptor database (GPCRdb).
In the cryo-EM structure of H1R with G_{q} (PDB ID: 7DFL), several key interactions were observed with the α₅ helix (F³⁴¹AAVKDTILQLNLKEYNLV³⁵⁹, SEQ ID NO: 13) upon receptor activation. For example, R125 in TM3 interacts with Y356 in the helix (cation-π interaction), residues in TM6 (K412) and H8 (N474) form a hydrogen bond with N357, and N352 in the α₅ helix interacts through a hydrogen bond with the backbone carbonyl of S128 in TM3 (Table 2) (Xia et al. 2021. Nat. Commun. 12: 1-9).

TABLE 2
Interacting residues of the α₅ helix from Gα₁₁
with H1R as defined by SEQ ID NO: 16.

| Residue of | Residue of | | | | Between | |
|---|---|---|---|---|---|---|
| Gα_{q}^{[1]} | H1R^{[1]} | BWN^{[2]} | Interaction | d (Å) | G_{q} | receptor |
| F³⁴¹ | P¹³² | P^{34.50} | VDW | 4.0 | m-Ar | β |
| A³⁴² | / | / | | | | |
| A³⁴³ | / | / | | | | |
| V³⁴⁴ | / | / | | | | |
| K³⁴⁵ | P¹³² | P^{34.50} | VDW | 3.4 | β | β |
| D³⁴⁶ | Q²¹⁹ | Q^{5.67} | HB | 2.5 | CO | NH₂ |
| T³⁴⁷ | / | / | | | | |
| I³⁴⁸ | L¹³³ | L^{34.51} | VDW | 3.6 | δ | δ |
| L³⁴⁹ | V/¹²⁹ | V^{3.54} | VDW | 4.4 | γ | γ |
| | Q²¹⁹ | Q^{5.67} | VDW | 3.7 | δ | NH₂ |
| | A²¹⁶ | A^{5.64} | VDW | 4.8 | δ | β |
| Q³⁵⁰ | R⁴⁰⁹ | R^{6.29} | VDW | 3.7 | CO | Guanidyl |
| L³⁵¹ | / | / | | | | |
| N³⁵² | S¹²⁸ | S^{3.53} | HB | 1.9 | NH₂ | CO^{BB} |
| L³⁵³ | R⁴⁰⁹ | R^{6.29} | VDW | 3.8 | δ | Guanidyl |
| | V²¹⁷ | V^{5.65} | VDW | 4.5 | δ | γ |
| | V¹²⁹ | V^{3.54} | VDW | 3.8 | δ | γ |
| K³⁵⁴ | / | / | | | | |
| E³⁵⁵ | T⁶⁰ | T^{2.37} | VDW | 3.6 | COO⁻ | OH |
| | R¹³⁹ | R^{34.57} | HB | 1.9 | CO | Guanidyl |
| Y³⁵⁶ | D¹²⁴ | D^{3.49} | VDW | 3.2 | OH | COO⁻ |
| | R¹²⁵ | R^{3.50} | ±π | 3.9 | m-Ar | γ |
| | | | (Splayed) | | | |
| | R¹³⁹ | R^{34.57} | HB | 2.5 | OH | Guanidyl |
| | N⁴⁷² | N^{8.47} | VDW | 3.5 | o-Ar | CO |
| N³⁵⁷ | K⁴¹² | K^{6.32} | HB | 1.8 | CO^{BB} | NH₂⁺ |
| | C⁴⁷¹ | C^{7.56} | HB | 1.9 | NH₂ | CO^{BB} |
| | N⁴⁷² | N^{8.47} | HB | 1.9 | NH₂ | CO^{BB} |
| | N⁴⁷⁴ | N^{8.48} | VDW | 3.5 | CO | NH₂ |
| L³⁵⁸ | A⁴¹³ | A^{6.33} | VDW | 4.3 | β | β |
| | Q⁴¹⁶ | Q^{6.36} | VDW | 4.0 | δ | β |
| | I⁴¹⁷ | L^{6.37} | VDW | 3.8 | δ | γ |
| | I²¹³ | I^{5.61} | VDW | 4.2 | δ | γ |
| | V²¹⁷ | V^{5.65} | VDW | 4.5 | δ | γ |
| V³⁵⁹ | K⁴¹² | K^{6.32} | HB | 1.7 | CO^{BB} | NH₃⁺ |
| | L⁴⁰⁵ | L^{6.25} | VDW | 3.8 | γ | γ |
| | R⁴⁰⁹ | R^{6.29} | VDW | 4.7 | γ | β |

^{[1]}Residue numbering based on cryo-EM structure (PDB: 7DFL).
^{[2]}Residue numbering according to the Ballesteros-Weinstein numbering from the sequence alignments in the GPCR database (GPCRdb).
The structure of 5-HT2AR was solved by cryo-EM in complex with an engineered G_{q} protein (mini-Gα_{q}-βγ heterotrimer) (PDB ID: 6WHA). The developed mini-G_{q} corresponded to the mini-Gₛ, with several point mutations, especially at the C-terminus. The activity of the mini-G_{q} was analyzed via a bioluminescence resonance energy transfer (BRET) assay and was comparable to the wild type Gα_{q}. In the cryo-EM structure of the active state receptor, several crucial hydrogen bonds were identified between the α₅ helix (F²²⁸NDCKDIILQMNLREYNLV²⁴⁶, SEQ ID NO: 14) and residues in the receptor, such as E242 with N107 in ICL2, Y243 with D172 in TM3, Q237 with N317 in TM6 and N244 with N384 in H8 (FIG. 3 and Table 3) (Kim et al. 2020. Cell 182: 1574-1588.e19).

TABLE 3
Interacting residues of the α₅ helix from
mini-G_{q} with 5-HT_{2A}R as defined by SEQ ID NO: 17.

| Residue of | Residue of | | | | between | |
|---|---|---|---|---|---|---|
| mini-G_{q}^{[1]} | 5-HT_{2A}R^{[1]} | BWN^{[2]} | Interaction | d (Å) | Mini-G_{q} | receptor |
| E²²⁸ | I¹⁸¹ | I^{34.51} | VDW | 4.0 | m-Ar | δ |
| N²²⁹ | / | / | | | | |
| D²³⁰ | / | / | | | | |
| C²³¹ | / | / | | | | |
| K²³² | I¹⁸¹ | I^{34.51} | VDW | 4.0 | β | δ |
| D²³³ | / | / | | | | |
| I²³⁴ | / | / | | | | |
| I²³⁵ | I¹⁸¹ | I^{34.51} | VDW | 4.1 | δ | γ |
| | S¹⁸⁴ | S^{34.54} | VDW | 3.5 | γ | OH |
| L²³⁶ | P¹⁸⁰ | P^{34.50} | VDW | 3.9 | γ | γ |
| | E³¹⁸ | E^{6.30} | VDW | 4.2 | δ | COO⁻ |
| Q²³⁷ | N³¹⁷ | N^{6.29} | HB | 1.8 | CO | NH₂ |
| M²³⁸ | / | / | | | | |
| N²³⁹ | A¹⁷⁶ | A^{3.53} | HB | 1.9 | NH | CO^{BB} |
| | H¹⁸³ | H^{34.53} | VDW | 3.1 | NH₂ | N(Ar) |
| L²⁴⁰ | I¹⁷⁷ | I^{3.54} | VDW | 3.9 | δ | δ |
| | L²⁶¹ | L^{5.65} | VDW | 4.4 | δ | δ |
| | A³²¹ | A^{6.33} | VDW | 4.2 | δ | β |
| R²⁴¹ | N³¹⁷ | N^{6.29} | VDW | 3.7 | Guanidyl | NH |
| E²⁴² | N¹⁰⁷ | N^{2.37} | HB | 2.2 | CO | NH₂ |
| Y²⁴³ | T¹⁰⁹ | T^{2.39} | VDW | 4.2 | m-Ar | OH |
| | D¹⁷² | D^{3.49} | HB | 1.8 | OH | COO⁻ |
| | R¹⁷³ | R^{3.50} | ±π (Splayed) | 4.3 | o-Ar | δ |
| | A¹⁷⁶ | A^{3.53} | VDW | 3.4 | m-Ar | β |
| | H¹⁸³ | H^{34.53} | VDW | 4.4 | m-Ar | CH(Arom) |
| N²⁴⁴ | N³⁸⁴ | N^{8.47} | HB | 1.8 | CO | NH₂ |
| L²⁴⁵ | R¹⁷³ | R^{3.50} | VDW | 4.0 | δ | Guanidyl |
| | A³²¹ | A^{6.33} | VDW | 4.1 | β | β |
| | V³²⁴ | V^{6.36} | VDW | 3.9 | δ | γ |
| | L³²⁵ | L^{6.37} | VDW | 3.9 | δ | δ |
| V²⁴⁶ | K³²⁰ | K^{6.35} | VDW | 3.9 | γ | γ |
| | N³¹⁷ | N^{6.29} | VDW | 3.8 | γ | CO |

^{[1]}Residue numbering based on cryo-EM structure (PDB: 6WHA).
^{[2]}Residue numbering according to the Ballesteros-Weinstein numbering from the sequence alignments in the GPCR database (GPCRdb).
Comparison of the different cryo-EM structures of the G_{q/11}-coupled receptors with their (mini-)Gα subunit, showed an overall similar engagement of the C-terminal as helix from the G protein. Similarly to the Gₛ protein, the α₅ helix of Gα_{q/11} and mini-G_{q} interacts with the receptor through one face only, with a crucial participation of the last 4-5 amino acids, forming a reverse turn at the Gα_{q/11}/mini-Gq proteins' C-terminus (data not shown). Therefore, the α₅ helix was identified as a key epitope to design peptidomimetics able to mimic the Gα_{q/11} subunit.

Two series were designed based on the cs helix of the G_{q/11} protein (SBL-GQ-01 to SBL-GQ-06) and based the α₅ helix of mini-G_{q} (SBL-GQ-07 to SBL-GQ-12) (Table 4).

TABLE 4
Synthesized (mini-)G_{q/11} mimics and their analytical data. (HPLC purity > 97%)

| | | | | Molecular | | tᵣ | | |
|---|---|---|---|---|---|---|---|---|
| | | SEQ ID | | weight | | HPLC | HRMS ([M + 2H]²⁺) | |
| Compound | Sequence | NO: | Formula | (g/mol) | Yield | (min) | Found | Calculated |
| SBL- | Ac-FAAVKDTILQLNLKEYNLV-OH | 1 | C₁₀₄H₁₆₈N₂₄O₃₀ | 2233.24 | 17 | 2.34 | 2234.2456 | 2234.2437 |
| GQ-01 | | | | | | | [M + H]⁺ | [M + H]⁺ |
| SBL- | Ac-KKKFAAVKDTILQLNLKEYNLV-OH | 2 | C₁₂₂H₂₀₄N₃₀O₃₃ | 2617.52 | 19 | 2.05 | 1309.7684 | 1309.7682 |
| GQ-02 | | | | | | | | |
| SBL- | Ac-FAAVKDTILQLNLKEYNChaV-OH | 3 | C₁₀₇H₁₇₂N₂₄O₃₀ | 2273.27 | 7 | 2.43 | 1137.6356 | 1137.6414 |
| GQ-03 | | | | | | | | |
| SBL- | Ac-KKKFAAVKDTILQLNLKEYNChaV-OH | 4 | C₁₂₅H₂₀₈N₃₀O₃₃ | 2657.55 | 14 | 2.14 | 1329.782 | 1329.7838 |
| GQ-04 | | | | | | | | |
| SBL- | Ac-KKKFA_{c}[PraVKDAzk]ILQLNLKEYNLV- | 5 | C₁₂₆H₂₀₇N₃₃O₃₂ | 2694.56 | 12 | 2.18 | 1348.2821 | 1348.2871 |
| GQ-05 | OH | | | | | | | |
| SBL- | Ac- | 6 | C₁₂₉H₂₁₁N₃₃O₃₂ | 2734.59 | 33 | 2.24 | 1368.3019 | 1368.3027 |
| GQ-06 | KKKFA_{c}[PraVKDAzk]ILQLNLKEYNChaV- | | | | | | | |
| | OH | | | | | | | |
| SBL- | Ac-FNDCKDIILQMNLREYNLV-OH | 7 | C₁₀₅H₁₆₇N₂₇O₃₂S₂ | 2382.17 | 7 | 2.23 | 1192.0874 | 1192.0934 |
| GQ-07 | | | | | | | | |
| SBL- | Ac-KKKFNDCKDIILQMNLREYNLV-OH | 8 | C₁₂₃H₂₀₃N₃₃O₃₅S₂ | 2766.46 | 11 | 2.02 | 1384.2404 | 1384.2358 |
| GQ-08 | | | | | | | | |
| SBL- | Ac-FNDCKDIILQMNLREYNChaV-OH | 9 | C₁₀₈H₁₇₁N₂₇O₃₂S₂ | 2422.2 | 3 | 2.31 | 1212.1083 | 1212.1091 |
| GQ-09 | | | | | | | | |
| SBL- | Ac-KKKFNDCKDIILQMNLREYNChaV-OH | 10 | C₁₂₆H₂₀₇N₃₃O₃₅S₂ | 2806.49 | 11 | 2.10 | 1404.2556 | 1404.2515 |
| GQ-10 | | | | | | | | |
| SBL- | Ac- | 11 | C₁₂₄H₂₀₂N₃₆O₃₃S₂ | 2787.47 | 10 | 2.09 | 1394.7468 | 1394.7417 |
| GQ-11 | KKKFN_{c}[PraCKDAzk]ILQMNLREYNLV- | | | | | | | |
| | OH | | | | | | | |
| SBL- | Ac- | 12 | C₁₂₇H₂₀₆N₃₆O₃₃S₂ | 2827.5 | 11 | 2.16 | 1414.7513 | 1414.7573 |
| GQ-12 | KKKFN_{c}[PraCKDAzk]ILQMNLREYNChaV- | | | | | | | |
| | OH | | | | | | | |

All peptides have been synthesized with a C-terminal carboxylic acid due to its significant importance for interaction with the receptor.In the study of the Gₛ mimetics, the switch from C-terminal carboxylic acid to C-terminal amide resulted in a drop in agonist affinity for the receptor (PCT/EP2021/086733).

To prevent any disruption of the native contacts with the receptor, a tether was inserted at the non-interacting side of the helix. A triazole bridge was selected. Therefore, the non-interacting residues were replaced by a propargylglycine (Pra) and an azidolysine (Azk) in i (position 3) and i+4 (position 7), to allow a single turn triazole stapling (SBL-GQ-05/06/11/12).

Furthermore, since the penultimate leucine seems to be a highly conserved residue amongst the G proteins, and responsible for crucial interactions with the receptor (FIGS. 2 and 3), it was substituted by the Cha residue (SBL-GQ-03/04/06/09/10/12).

Moreover, N-terminal polylysine (KKK) was also added to the sequence to prevent any solubility issues, especially in the case of stapled mimetics. However, for linear peptides, analogues with (SBL-GQ-02/04/08/10) and without trilysine (SBL-GQ-01/03/07/09) were synthesized.

Because there was no difference between N-terminal acetylated and non-acetylated Gₛ analogues, only the acetylated version of the (mini-)G_{q/11} sequences have been tested.

The peptides were prepared using Fmoc-based SPPS with the assistance of an automated synthesizer and their characterization is to be found in Table 4. The solid phase synthesis was performed on a Wang resin, already preloaded with valine, and followed by repeated cycles of amino acid deprotection and coupling using DIC and Oxyma as coupling mixture. After acetylation of the N-terminus with acetic anhydride and DIPEA, the α-helical conformation was stabilized by peptide ‘stapling’ between the side chains of Pra (at position 3) and Azk (at position 7) through a copper-catalyzed azide-alkyne cycloaddition using CuBr.

### Example 2: Pharmacological Evaluation of the G_{q/11} Protein-Based Peptidomimetics by a Radioligand Displacement Assay

To evaluate the capacity of the synthesized (mini-)G_{q/11} mimetics to stabilize a G_{q/11}-mediated receptor, radioligand binding assays (RLA) on the muscarinic acetylcholine 1 receptor were performed. Unfortunately, the linear (mini-)G_{q/11} mimetics, without the trilysine (SBL-GQ-01/03/07/09), were not soluble in aqueous buffer and were therefore not tested by RLA.

The radioligand binding experiments were performed to test the binding affinity of an agonist (acetylcholine chloride, which binds to the extracellular side) for the M1R receptor in presence and absence of the peptidomimetic. Therefore, the receptor bound to a radioactively labeled neutral antagonist ([³H]—N-methyl scopolamine) was incubated with different concentrations of the agonist on a 96-well plate. In this setting, the radioligand and agonist competed for the extracellular binding site of the receptor. At low agonist concentration a high percentage of radioligand was still bound to the receptor. When the agonist concentration was increased, radioligand was displaced by the agonist. If in presence of the (mini-)G_{q/11} peptidomimetic, a lower amount of radioligand was able to bind the receptor, at the same agonist concentration, the peptidomimetic was able to stabilize the receptor in its active conformation.

TABLE 5
Half maximal inhibitory concentration (IC₅₀) and shift of the synthesized (mini-)G_{q/11} mimics
as determined by RLA. ^{[1]}_{c}[ ] Cyclic peptide. ^{[2]}The IC₅₀ represents the affinity of the
agonist for the M1 receptor and are shown as means ± SEM with number of replicates indicated
between brackets, each performed in duplicate. ^{[3]}The selectivity is quantified by
the shift (averaged value).

| | | SEQ | | | |
|---|---|---|---|---|---|
| | | ID | | IC₅₀ | |
| | Compound | NO: | Sequence^{[1]} | (μM)^{[2]} | Shift^{[3]} |
| Gα_{q/11} | α₅ | 13 | F¹A²A³V⁴K⁵D⁶T⁷I⁸L⁹Q¹⁰L¹¹N¹²L¹³K¹⁴E¹⁵Y¹⁶N¹⁷L¹⁸V¹⁹ | | |
| | SBL-GQ-01 | 1 | Ac-FAAVKDTILQLNLKEYNLV-OH | ND | ND |
| | SBL-GQ-02 | 2 | Ac-KKKFAAVKDTILQLNLKEYNLV-OH | 473.8 (1) | 2 |
| | SBL-GQ-03 | 3 | Ac-FAAVKDTILQLNLKEYNChaV-OH | ND | ND |
| | SBL-GQ-04 | 4 | Ac-KKKFAAVKDTILQLNLKEYNChaV-OH | 212.7 (1) | 4 |
| | SBL-GQ-05 | 5 | Ac-KKKFA_{c}[PraVKDAzk]ILQLNLKEYNLV-OH | 330.3 (1) | 1 |
| | SBL-GQ-06 | 6 | Ac-KKKFA_{c}[PraVKDAzk]ILQLNLKEYNChaV-OH | 54.4 ± 20 | 8 |
| Mini-G_{q} | α₅ | 14 | F¹N²D³C⁴K⁵D⁶I⁷I⁸L⁹Q¹⁰M¹¹N¹²L¹³R¹⁴E¹⁵Y¹⁶N¹⁷L¹⁸V¹⁹ | | |
| | SBL-GQ-07 | 7 | Ac-FNDCKDIILQMNLREYNLV-OH | ND | ND |
| | SBL-GQ-08 | 8 | Ac-KKKFNDCKDIILQMNLREYNLV-OH | 412.7 (1) | 2 |
| | SBL-GQ-09 | 9 | Ac-FNDCKDIILQMNLREYNChaV-OH | ND | ND |
| | SBL-GQ-10 | 10 | Ac-KKKFNDCKDIILQMNLREYNChaV-OH | 409.1 (1) | 2 |
| | SBL-GQ-11 | 11 | Ac-KKKFN_{c}[PraCKDAzk]ILQMNLREYNLV-OH | 57.7 (1) | 7 |
| | SBL-GQ-12 | 12 | Ac-KKKFN_{c}[PraCKDAzk]ILQMNLREYNChaV-OH | 13.7 ± 6.1 | 34 |

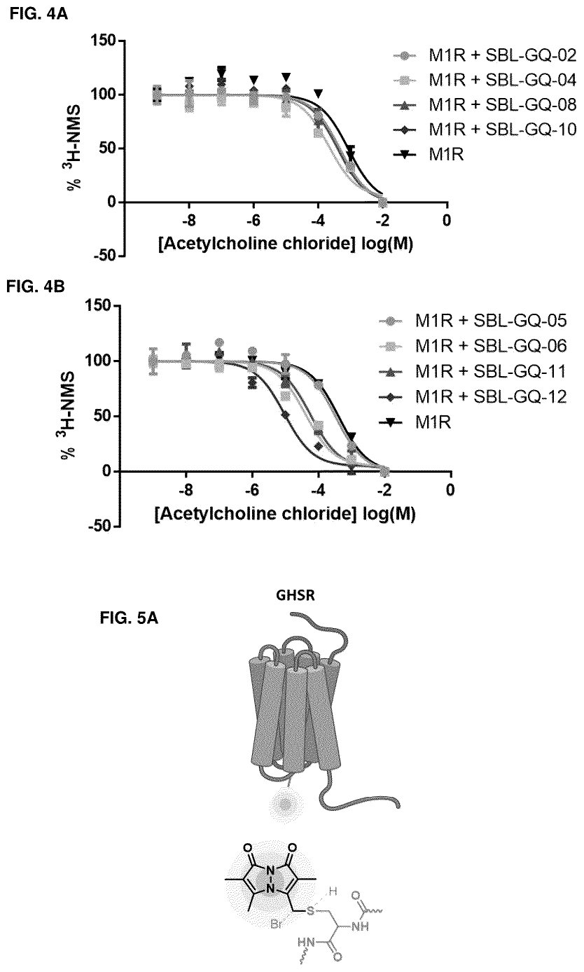

No significant increase of the agonist affinity was observed when performing the radioligand binding assays with the linear (mini-)G_{q/11} mimetics (FIG. 4A and Table 5). While a slight increase in agonist affinity was found for SBL-GQ-06 and SBL-GQ-11, a more significant and promising stabilization of the receptor was observed for SBL-GQ-12 (FIG. 4B and Table 5). The radioligand binding experiment was repeated two times for these sequences and similar results were obtained for both repeats. From this set of analogues, the mini-G_{q} derived peptidomimetic, SBL-GQ-12, was responsible for the highest increase in agonist affinity for the receptor, in comparison to the peptidomimetic based on Gα_{q/11} (SBL-GQ-06).

Interestingly, and similarly to the Gₛ peptidomimetics (PCT/EP2021/086733), the Cha residue at the penultimate position appeared to play a beneficial role in the stabilization of the G_{q/11}-coupled receptors, since a higher agonist affinity was observed for the stapled peptides containing a Cha residue compared to their analogue without (SBL-GQ-06 vs SBL-GQ-05 and SBL-GQ-12 vs SBL-GQ-11). Without wishing to be bound by any theory, replacement of the penultimate leucine (L358) of Gα₁₁, which forms Van der Waals (VDW) interactions with A363 and L367 in the receptor (FIG. 2), with a Cha residue that can be regarded as an extended leucine, could reduce the distance to A363 and L367 and strengthen the interactions with the receptor.

### Example 3: Pharmacological Evaluation of the G_{q/11} Protein-Based Peptidomimetics by a Bimane Fluorescence Assay

Next to RLA, another assay can be used to follow the conformational changes of GPCRs upon ligand binding, namely the bimane fluorescence assay. This assay was performed on the purified ghrelin receptor. The growth hormone secretagogue receptor (GHSR), commonly called ghrelin receptor, is a GPCR that can signal through multiple pathways, including the G_{q} protein. It is known to regulate energy homeostasis and body weight (Damian et al. 2021. Nat. Commun. 12:1-15).

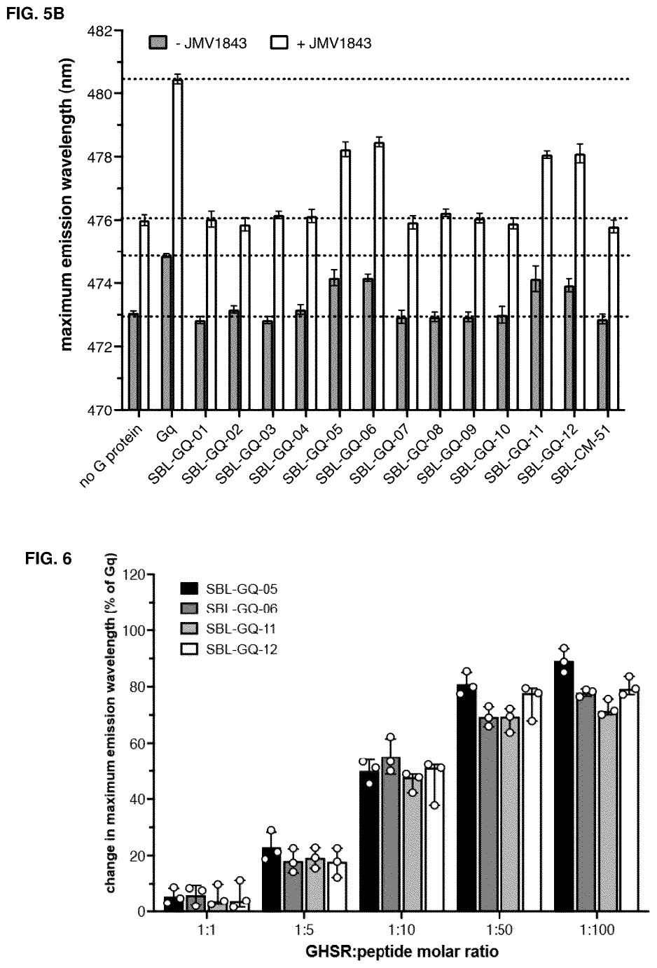

The bimane assay is used on GPCRs to detect conformational changes associated with receptor activation. This assay functions through the labelling of a cysteine, which is one of the least frequently occurring amino acids in proteins. Additionally, the majority of the extracellular cysteines form disulfide bonds, which, together with the transmembrane cysteines, are inert to thiol-reactive reagents. Additionally, some of the intracellular cysteines in the C-terminal tail may carry post-translational modifications which reduces the number of reactive cysteines in the receptor (Tian et al. 2017. Chem. Rev. 117:186-245). For GHSR, the remaining reactive cysteine residues C146 (ICL2) and C304 (extracellular end of TM7) were replaced by serines (cysmin mutant, i.e. mutant with minimal cysteines), to allow the monobromobimane (MB) fluorescent probe to be specifically attached to Cys255 in the lower part of TM6 (FIG. 5a) (Damian et al. 2021). In this assay, bimane is chosen because of its small size and sensitivity to the polarity of its environment (Yao et al. 2006. Nat Chem. Biol. 2:417-422). Binding of a ligand to the receptor, causes a conformational change and outward movement of TM6 that places bimane in a more solvent-exposed position, which alters its maximum emission wavelength.

The series of (mini-)G_{q/11} peptidomimetics of example 1 were tested on purified bimane labeled ghrelin receptor in the presence and absence of a ghrelin receptor full agonist: JMV1843 (Guerlavais et al. 2003. J. Med. Chem. 46:1191-1203) (FIG. 5b). A control assay with and without G_{q} protein (Gα_{q}β₁γ₂ heterotrimer) was performed to determine the maximum emission wavelength of the active (with agonist and G_{q} protein) and basal (without agonist and G_{q} protein) conformation. Binding of the agonist in the presence of the G_{q} protein induced a significant change in bimane emission wavelength (±480.5 nm). The wavelength decreased in the absence of the G_{q} protein (±476 nm), indicating that binding of the G protein caused a conformational change that influenced the environment of the bimane fluorophore. The (mini-)G_{q/11} peptidomimetics were tested for possible effects on the bimane labeled receptor alone and in the presence of JMV1843. Binding of the 4 stapled peptidomimetics (SBL-GQ-05/06/11/12) in presence of the agonist resulted in an increase of the maximum emission wavelength (±478 nm), but lower as with the native G_{q} protein. In contrast to the results of the radioligand binding experiments (example 2), no significant difference was observed between the mini-G_{q} and G_{q/11} derived peptidomimetics, neither between the peptides containing, or not, a Cha residue. The linear (mini-)G_{q/11} (SBL-GQ-01/02/03/04/07/08/09/10) mimetics were not able to induce a conformational change in the receptor, with a maximum emission wavelength comparable to the control assay without G_{q} protein.

The data were normalized to the maximal effect triggered by the G_{q} protein and are represented in FIG. 6. Interestingly, the four peptidomimetics showed a dose response effect with an almost maximal effect for 1:50 and 1:100 (GHSR:peptide). While no significant difference was observed between the G_{q/11} and mini-G_{q} derived peptidomimetics (with and without Cha residue), a slightly higher emission wavelength was observed for SBL-GQ-05 at the highest peptide concentrations, indicating a more pronounced conformational change corresponding to approximately 90% of the effect of the G_{q} protein.

Whereas in the radioligand assay the mini-G_{q} derived peptidomimetic with Cha residue (SBL-GQ-12) showed the highest stabilization of the receptor, in the bimane assay the stapled peptidomimetics caused an almost equivalent conformational change, with even a slightly higher effect for the G_{q/11} derived peptidomimetic without Cha residue (SBL-GQ-05).

### Example 4: Optimization of the G_{q/11} and Mini-G_{q} Derived Peptidomimetics

A first possible optimization of the G_{q/11} and mini-G_{q} derived peptidomimetics of example 1 is to replace the last valine with an acidic amino acid such as Asp or Glu to target basic residues nearby (M1R as defined by SEQ ID NO: 15: T215, R218, K361 and K362; H1R as defined by SEQ ID NO: 16: L405(BB), R409 and K412; 5-HT_{2A}R as defined by SEQ ID NO: 17: N317, K320, N384 and K385). Their D-counterpart (D-Asp and D-Glu) can also be introduced to bring the amino acid in the seemingly more appropriate orientation for additional interactions.

Another strategy is to perform a screening of aromatic residues (Phe, Tyr and Trp) at the penultimate position in order to target a receptor arginine in close proximity (M1R as defined by SEQ ID NO: 15: R123; H1R as defined by SEQ ID NO: 16: R125; 5-HT2AR as defined by SEQ ID NO: 17: R173), to induce a cation-π interaction. Additionally, the penultimate leucine is substituted by a glutamic acid to create a hydrogen bond with the proximal arginine.

Furthermore, it was observed that the C-terminal asparagine in the α₅ helix, was interacting close to a cysteine residue, located in the binding pocket of M1R and H1R. A gateway to peptidomimetic-receptor conjugates is to replace asparagine by a cysteine, to allow a covalent disulfide bridge between the G_{q/11} mimetic and the receptor (M1R as defined by SEQ ID NO: 15: C421; H1R as defined by SEQ ID NO: 16: C471). This can be performed on the G_{q/11} derived peptides which do not contain a cysteine residue at their N-terminus, to avoid intramolecular cyclization. Or: the N-terminal Cys residue of mini-G_{q} derived peptides can be replaced to avoid such a cyclization.

Additionally, it has been observed that the Tyr residue, 4th last position in the α₅ helix, was surrounded by rather acidic or polar groups. To keep the cation-n interaction with the proximal arginine (M1R as defined by SEQ ID NO:15: R123; H1R as defined by SEQ ID NO: 16: R125; 5-HT2AR as defined by SEQ ID NO:17: R173) and increase the number of hydrogen bonds, it is replaced by a Phe(4′-guanidino) (to target M1R as defined by SEQ ID NO: 15: N60, D122, S126 and R123; H1R as defined by SEQ ID NO: 16: D124, S128, N472 and R125; 5-HT_{2A}R as defined by SEQ ID NO: 17: T109, D172 and R173).

Finally, the aspartic acid (5th last) is substituted by a homoglutamic acid to decrease the distance and strengthen the interactions with the residues in the binding pocket (M1R as defined by SEQ ID NO: 15: N60, N61 and N422; H1R as defined by SEQ ID NO: 16: T60, R139 and N472; 5-HT_{2A}R as defined by SEQ ID NO: 17: N107, N187 and R189).

### Example 5: Generation of Cell-Permeable Gα_{q/11} Peptidomimetics

The cationic cell-penetrating peptide (CPP) Argₛ(SEQ ID NO 36) (SBL-GQ-15) and Arg₄ (SEQ ID NO:37) (SBL-GQ-014) and the amphipathic CPP RW9 (SEQ ID NO:39) (SBL-GQ-25) were attached to the Gq/11 peptidomimetic SBL-GQ-05 to increase its cell-permeability for investigating intracellular interactions with the ghrelin receptor. In addition, to allow investigation of the permeability by internalization assays and microscopy, different fluorophores (DY-647P1, Pacific Blue, Sulfocyanine 3 and Sulfocyanine 5) were attached to the N-terminus of SBL-GQ-05 and the CPP-modified SBL-GQ-05.

Table 6 summarizes the modified Gα_{q/11} peptidomimetics that were synthesized by solid-phase peptide synthesis (SPPS).

TABLE 6
Gα_{q/11} peptidomimetics

| | | SEQ |
|---|---|---|
| | | ID |
| Compound | Sequence/structure | NO: |
| SBL-GQ-04 | Ac-KKKFAAVKDTILQLNLKEYNChaV-OH | 4 |
| SBL-GQ-05 | 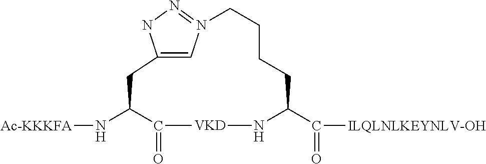 | 5 |
| | Ac-KKKFA_{c}[PraVKDAzk]ILQLNLKEYNLV-OH | |
| SBL-GQ-13 | 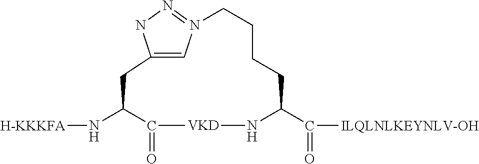 | 41 |
| | H-KKKFA_{c}[PraVKDAzk]ILQLNLKEYNLV-OH | |
| SBL-GQ-14 | 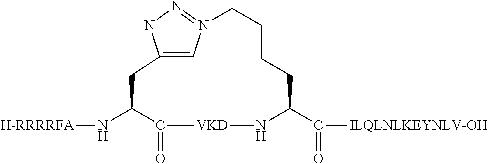 | 42 |
| | H-RRRRFA_{c}[PraVKDAzk]ILQLNLKEYNLV-OH | |
| SBL-GQ-15 | 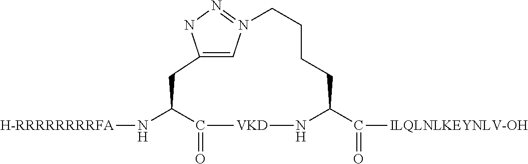 | 43 |
| | H-RRRRRRRRFA_{c}[PraVKDAzk]ILQLNLKEYNLV-OH | |
| SBL-GQ-16 | 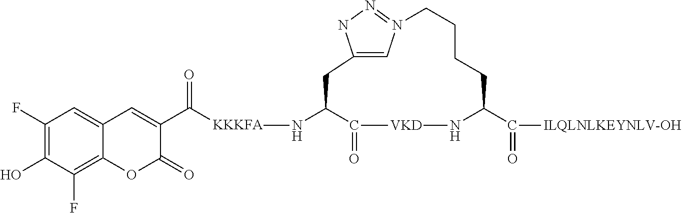 | 41 |
| | Pacific Blue-KKKFA_{c}[PraVKDAzk]ILQLNLKEYNLV-OH | |
| SBL-GQ-17 | 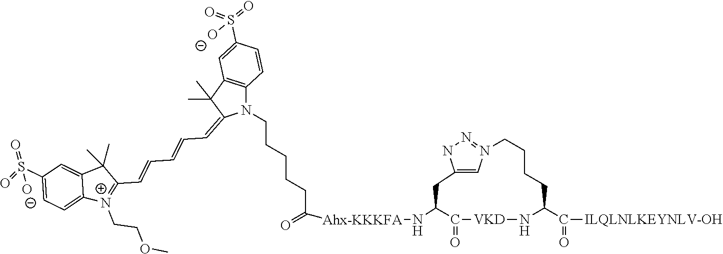 | 44 |
| | DY647-Ahx-KKKFA_{c}[PraVKDAzk]ILQLNLKEYNLV-OH | |
| SBL-GQ-18 | 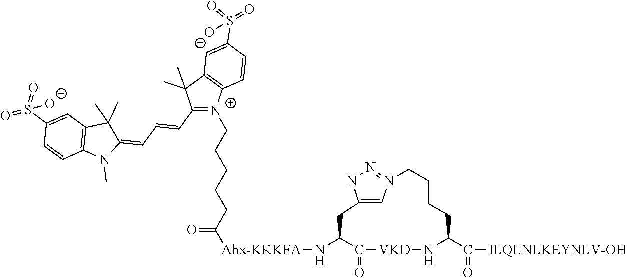 | 44 |
| | SulfoCy3-Ahx-KKKFA_{c}[PraVKDAzk]ILQLNLKEYNLV-OH | |
| SBL-GQ-19 | 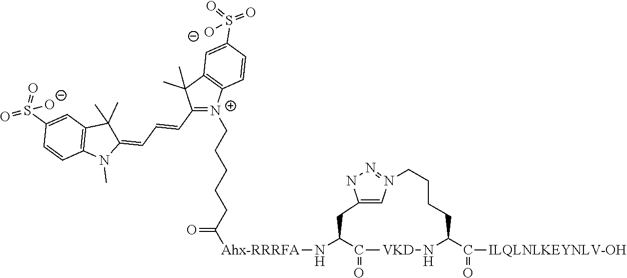 | 45 |
| | SulfoCy3-Ahx-RRRRFA_{c}[PraVKDAzk]ILQLNLKEYNLV-OH | |
| SBL-GQ-20 | 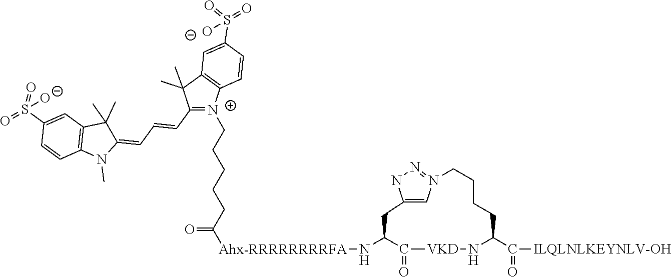 | 46 |
| | SulfoCy3-Ahx-RRRRRRRRFA_{c}[PraVKDAzk]ILQLNLKEYNLV-OH | |
| SBL-GQ-21 | 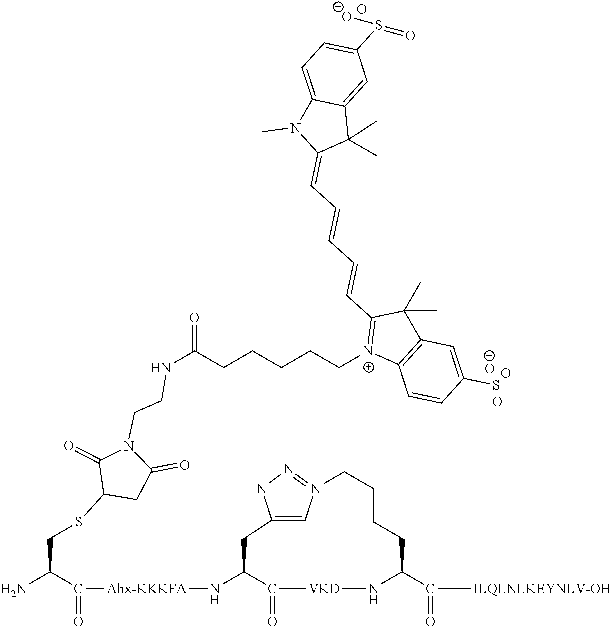 | 47 |
| | H-C(SulfoCy5)-Ahx-KKKFA_{c}[PraVKDAzk]ILQLNLKEYNLV-OH | |
| SBL-GQ-22 | 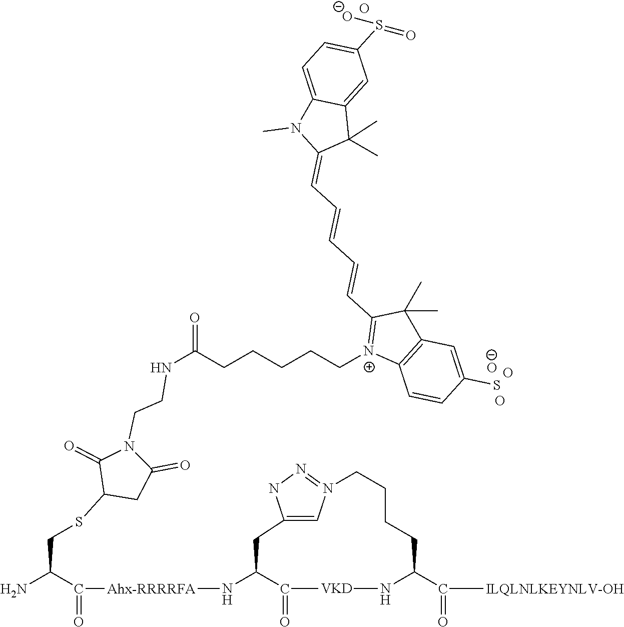 | 48 |
| | H-C(SulfoCy5)-Ahx-RRRRFA_{c}[PraVKDAzk]ILQLNLKEYNLV-OH | |
| SBL-GQ-23 | 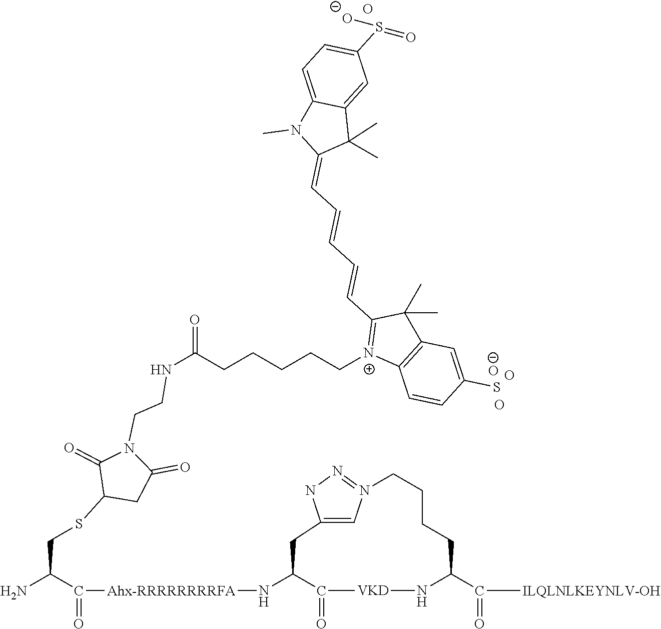 | 49 |
| | H-C(SulfoCy5)-Ahx-RRRRRRRRFA_{c}[PraVKDAzk]ILQLNLKEYNLV-OH | |
| SBL-GQ-24 | 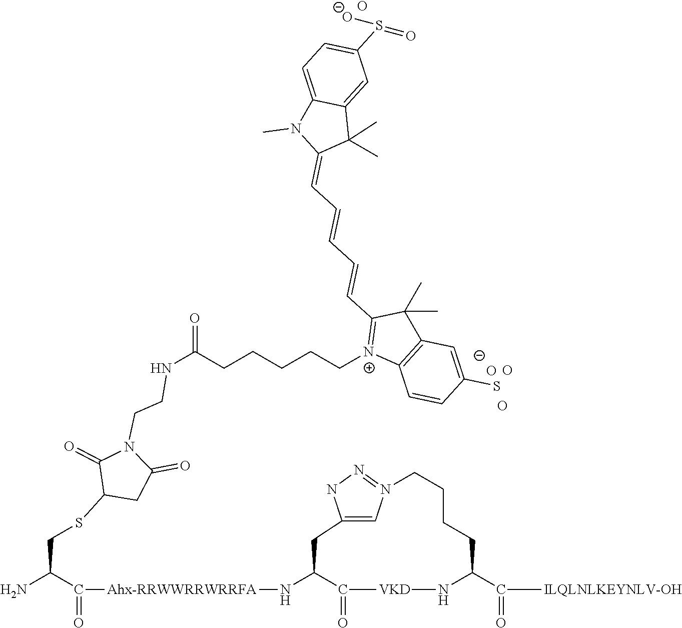 | 50 |
| | H-C(SulfoCy5)-Ahx-RRWWRRWRRFA_{c}[PraVKDAzk]ILQLNLKEYNLV-OH | |
| SBL-GQ-25 | 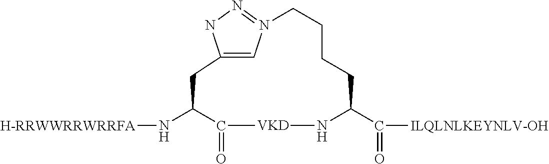 | |
| | H-RRWWRRWRRFA_{c}[PraVKDAzk]ILQLNLKEYNLV-OH | |

### Example 6: IP-One G_{q} Assay

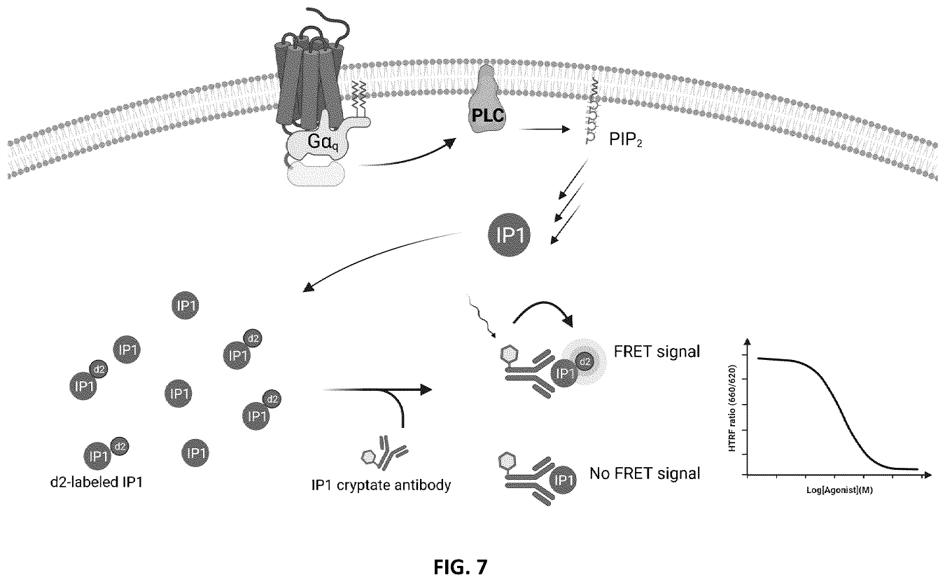

To investigate whether G_{q/11} peptidomimetics are also able to stabilize a G_{q/11}-mediated receptor in a cellular context (the radioligand displacement assay was performed on membrane extracts and the bimane fluorescent assay was performed on purified ghrelin receptor reconstituted into lipidated nanodiscs), an IP-One G_{q} assay was performed. The IP-One G_{q} assay detects the accumulation of inositol monophosphate (IP1), a metabolite produced following phospholipase C activation. A schematic representation of the assay is shown in FIG. 7.

The non-fluorescent G_{q/11} peptidomimetics of Example 5 were tested in the IP-One G_{q} assay according to the manufacturers instructions.

The assays were performed on 384-well plates containing HEK293 cells that overexpress the ghrelin receptor. First, the G_{q/11} peptidomimetics were incubated during 1 h to allow penetration in the cells. Afterwards, the receptor agonist MK0677 was added in varying concentrations to activate the ghrelin receptor and induce inositol monophosphate production (native unlabeled IP1). After 45 min of incubation, the cells were lysed and d2-labeled IP1 (acceptor, 665 nm) was exogenously added to compete with the native unlabeled IP1 for binding with the anti-IP1-Cryptate (donor, 620 nm). Fluorescence was measured and the fluorescence ratio (665 nm/620 nm) was determined. SBL-GQ-04, which demonstrated no stabilization of G_{q/11} protein-coupled receptors in the RLA and bimane fluorescence assays (Examples 2 and 3) was used as a negative control.

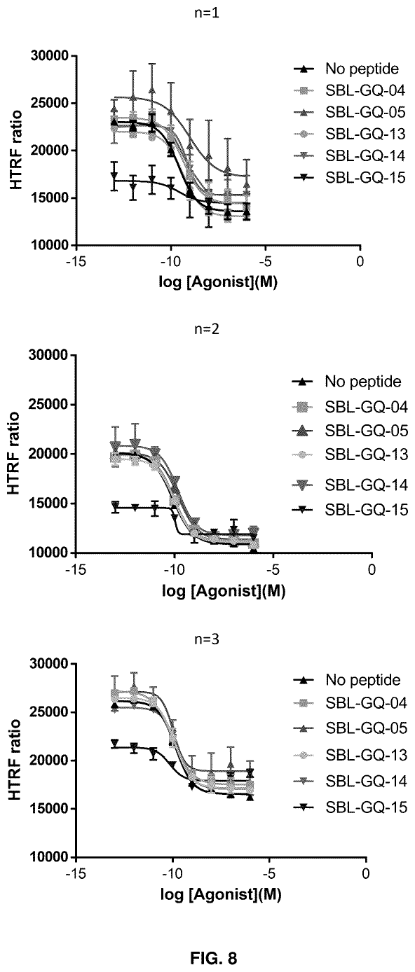

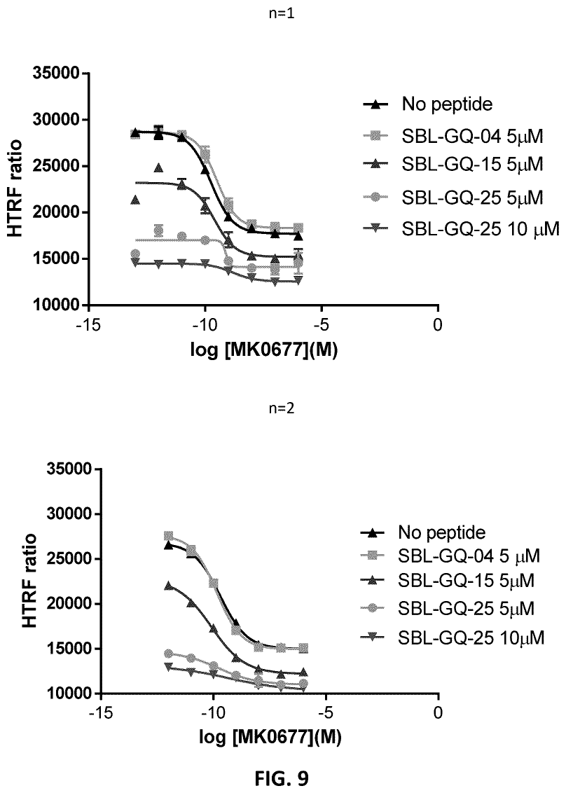

Almost no difference in receptor activation was obtained when the assay was performed in the presence of the G_{q/11} peptidomimetics SBL-GQ-04, SBL-GQ-05, SBL-GQ-13 and SBL-GQ-14 compared to the absence of a G_{q/11} peptidomimetic (FIG. 8). In the presence of SBL-GQ-15 a decrease in fluorescence ratio was observed (FIG. 8), indicating that the G_{q/11} peptidomimetic was able to enhance activation of the receptor to induce IP1 production. An even stronger decrease in fluorescence ratio was observed when the assay was performed in the presence of the G_{q/11} peptidomimetic SBL-GQ-25 (FIG. 9) at 5 and 10 μM.

TABLE 7
Half maximal effective concentration (EC₅₀) of the MK0677 agonist
in the absence or presence of the indicated G_{q/11} peptidomimetic as
determined from the IP-One G_{q} assay shown in FIG. 8. The IP-One G_{q}
assay was performed in the absence of any G_{q/11} peptidomimetic
or in the presence of the indicated G_{q/11} peptidomimetics
at 10 μM. The assays were performed three times (n = 3), in triplicate.

EC50 (M)

| | n = 1 | n = 2 | n = 3 |
|---|---|---|---|
| No peptide | 2.64e−10 | 8.12e−11 | 1.434e−10 |
| SBL-GQ-04 | 4.42e−10 | 9.46e−11 | 9.87e−11 |
| SBL-GQ-05 | 9.99e−10 | 1.53e−10 | 1.10e−10 |
| SBL-GQ-13 | 4.42e−10 | 1.01e−10 | 1.31e−10 |
| SBL-GQ-14 | 6.96e−10 | 1.01e−10 | 1.87e−10 |
| SBL-GQ-15 | 2.75e−10 | 1.07e−10 | 7.18e−11 |

TABLE 8
Half maximal effective concentration (EC₅₀) of the MK0677 agonist
in the absence or presence of the indicated G_{q/11} peptidomimetic as
determined from the IP-One G_{q} assay shown in FIG. 9. The IP-One G_{q}
assay was performed in the absence of any G_{g/11} peptidomimetic
or in the presence of the indicated G_{q/11} peptidomimetic
at 10 μM. The assays were performed three times (n = 3), in triplicate.

EC50 (M)

| | n = 1 | n = 2 |
|---|---|---|
| No peptide | 1.80e−10 | 2.00e−10 |
| SBL-GQ-04 5 μM | 3.18e−10 | 1.41e−10 |
| SBL-GQ-15 5 μM | 2.55e−10 | 8.07e−11 |
| SBL-GQ-25 5 μM | 7.82e−10 | 1.7e−10 |
| SBL-GQ-25 10 μM | 1.54e−9 | 4.65e−10 |

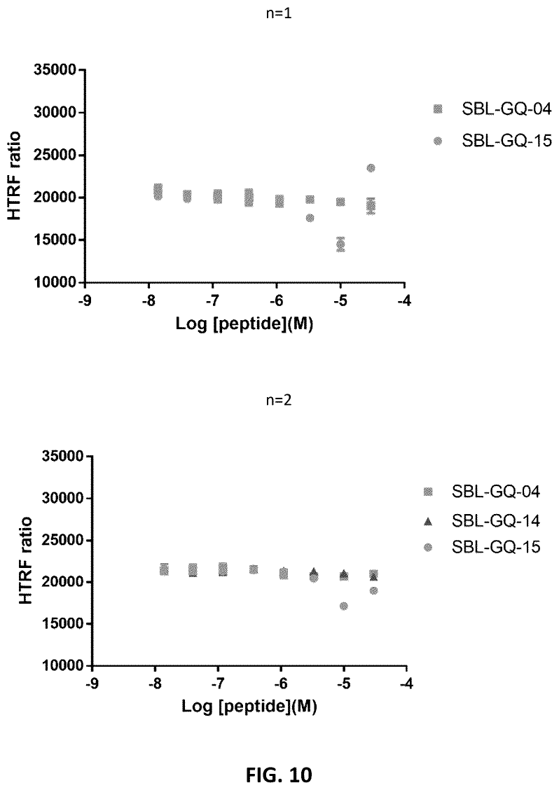

The IP-One Gq assay was also performed by a dose-response of the G_{q/11} peptidomimetic instead of varying the concentration of the agonist. The results correlated with the observations shown in FIGS. 8 and 9 (FIG. 10).

In conclusion, the results show that G_{q/11} peptidomimetics according to embodiments of the invention are able to stabilize G_{q/11} protein-coupled receptor and enter living cells.

### Example 7: Cell Internalization Assay

The cell permeability of the Sulfocyanine 5(SulfoCy5)-labeled G_{q/11} peptidomimetics of Example 5 were investigated on HEK293 cells overexpressing the ghrelin receptor by total fluorescence emission measurement. In this cell internalization assay, the HEK293 cells were incubated with the SulfoCy5-labeled G_{q/11} peptidomimetics at 5 and 10 μM. After incubation, the cells were washed multiple times with PBS and the remaining fluorescence was measured. As negative control, SulfoCy5-labeled dynorphin, a peptide targeting the κ-opioid receptor with no cell permeability properties, was used.

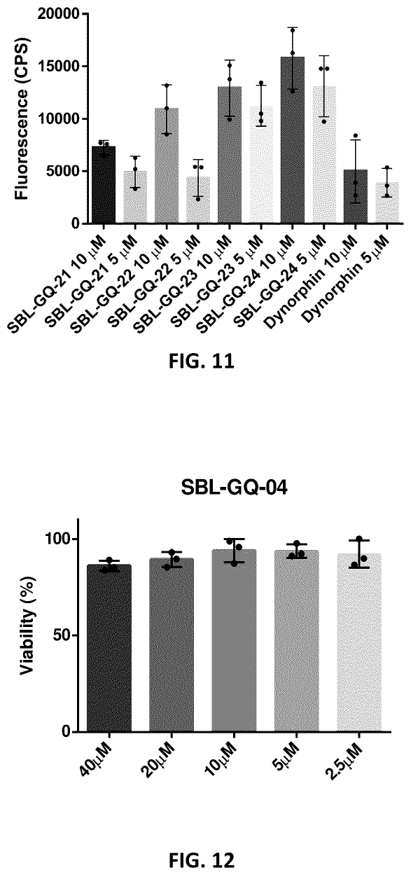

SBL-GQ-24 containing the RW9 CPP showed the highest capacity to cross the cell membrane, followed by SBL-GQ-23 containing the Argₛ CPP (FIG. 11). While a little internalization capacity was still observed for SBL-GQ-22 containing Arg₄ CPP, almost no internalization, comparable to dynorphin (negative control), was observed for SBL-GQ-21.

### Example 8: Assessment of Cell Permeability of G_{q/11} Peptidomimetics by Fluorescence Microscopy

The SulfoCy5-labeled G_{q/11} peptidomimetics of Example 5 were also analyzed by fluorescence microscopy to investigate their cell membrane permeability. HEK293 cells overexpressing the ghrelin receptor were seeded overnight on microscope slides. The cells were incubated with 5 μM of SulfoCy5-labeled G_{q/11} peptidomimetics for 3 h 45 min at 37° C., followed by Hoechst staining for visualizing the nucleus. The cell membrane was visualized by staining the cell surface ghrelin receptors with fluorescein.

The microscope images (data not shown) correlated with the results obtained in the cell internalizations assays of example 7: SulfoCy5-labeled G_{q/11} peptidomimetics entered cells that were incubated with SBL-GQ-23 and SBL-GQ-24, while very little or no SulfoCy5-labeled G_{q/11} peptidomimetics were observed inside cells incubated with SBL-GQ-22, SBL-GQ-21 and dynorphin (negative control).

### Example 9: Cytotoxicity

An MTT assay was used to assess the cytotoxicity of the non-fluorescent G_{q/11} peptidomimetics of Example 5. An MTT assay is a colorimetric test that evaluates the cell metabolic activity and reflects the cell viability.

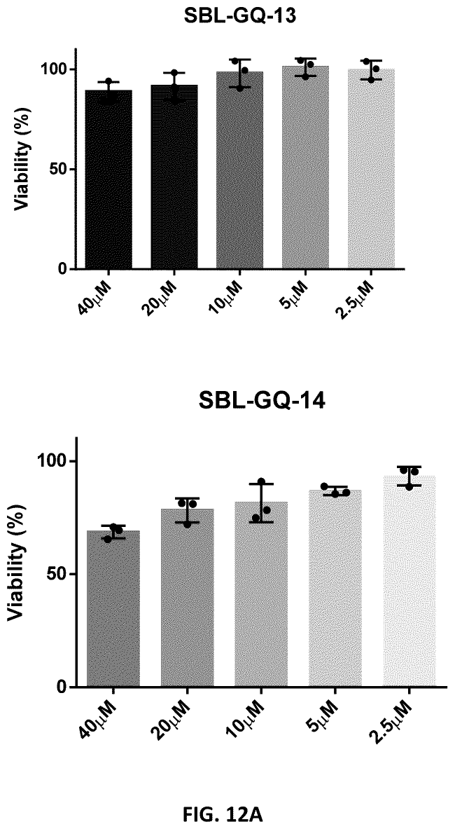

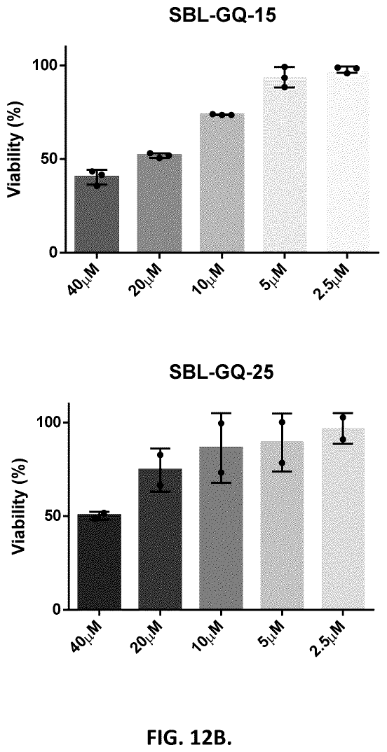

No cytotoxicity was observed for SBL-GQ-04 and SBL-GQ-13, even at higher concentrations (40 μM), while for SBL-GQ-15 and SBL-GQ-25 a cell viability of only ±50% was obtained at 40 μM (FIGS. 12A-12C). For SBL-GQ-15, a cell viability of ±70% was obtained at 10 μM and ±90% at 5 μM. For SBL-GQ-25 a cell viability of ±85% was observed for 10 μM and ±90% for 5 μM (FIGS. 12A-12C). Without wishing to be bound by any theory, the observed increase in cytotoxicity for higher concentrations of Arg₈ and RW9-containing G_{q/11} peptidomimetics may be due to a partial disruption of the cell membrane by these CPPs to allow the G_{q/11} peptidomimetics to pass the cellular membrane. For SBL-GQ-14, with less arginine residues in the CPP compared to SBL-GQ-15, only a slight decrease in viability was noticed at higher concentrations (FIGS. 12A-12C).

1. A G protein peptidomimetic or salt thereof comprising a sequence of the structure (XIV):
(XIV)
(SEQ ID NO: 18)
FX₂X₃X₄KDX₇ILQX₁₁NLX₁₄EYNX₁₈V
wherein X₂ is asparagine (N) or alanine (A);
wherein X₃ is selected from the group consisting of: an amino acid containing an azidated side-chain, an amino acid residue containing an alkynyl side-chain, an amino acid containing a carboxylic acid group side-chain, an amino acid containing an amine side-chain, an amino acid containing a thiol side-chain, and an olefinic amino acid;
wherein X₄ is cysteine (C) or valine (V);
wherein X₇ is selected from the group consisting of: an amino acid containing an azidated side-chain, an amino acid containing an alkynyl side-chain, an amino acid containing a carboxylic acid group side-chain, an amino acid containing an amine side-chain, an amino acid containing a thiol side-chain, and an olefinic amino acid;
wherein X₁₁ is methionine (M) or leucine (L);
wherein X₁₄ is arginine (R) or lysine (K); and
wherein X₁₈ is leucine (L), or an alanine analogue, phenylalanine (F), tyrosine (Y), or tryptophan (W), wherein the alanine analogue is a molecule resulting from the replacement of at least one hydrogen of an alanine by at least one moiety selected from the group comprising C₆₋₁₂ cycloalkyl, C₆₋₁₂ aryl, heteroaryl, and C₆₋₁₂ cycloalkenyl; each moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, and haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl;
wherein the peptidomimetic comprises a covalent tether formed from the reaction of the side-chain of X₃ with the side-chain of X₇, wherein X₇ is an amino acid containing an azidated side-chain and X₃ is an amino acid containing an alkynyl side-chain, wherein X₃ is an amino acid containing an azidated side-chain and X₇ is an amino acid containing an alkynyl side-chain, wherein X₃ is an amino acid containing a carboxylic acid group side-chain and X₇ is an amino acid containing an amine side-chain, wherein X₇ is an amino acid containing a carboxylic acid group side-chain and X₃ is an amino acid containing an amine side-chain, wherein X₃ and X₇ are olefinic amino acids, or wherein X₃ and X₇ are amino acids containing a thiol side-chain.

2. The G protein peptidomimetic or salt thereof of claim 1, comprising a sequence of the structure (XV) or (XVI):
(XV)
(SEQ ID NO: 19)
FNX₃CKDX₇ILQMNLREYNX₁₈V
(XVI)
(SEQ ID NO: 20)
FAX₃VKDX₇ILQLNLKEYNX₁₈V.

3. The G protein peptidomimetic or salt thereof of claim 1, wherein X₇ is an amino acid containing an azidated side-chain and X₃ is an amino acid containing an alkynyl side-chain or wherein X₃ is an amino acid containing an azidated side-chain and X₇ is an amino acid containing an alkynyl side-chain.

4. The G protein peptidomimetic or salt thereof of claim 1, wherein the amino acid containing an azidated side-chain is azidolysine (Azk) and wherein the amino acid containing an alkynyl side-chain is propargylglycine (Pra).

5. The G protein peptidomimetic or salt thereof of claim 1, wherein X₁₈ is leucine (L).

6. The G protein peptidomimetic or salt thereof of claim 1, wherein X₁₈ is a moiety of formula (Ia): 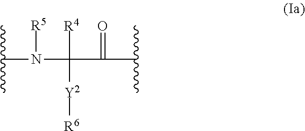
wherein:
R⁴ is hydrogen or C₁₋₆ alkyl;
R⁵ is hydrogen or C₁₋₆ alkyl;
R⁶ is a moiety selected from the group comprising C₆₋₁₂ cycloalkyl, C₆₋₁₂ aryl, heteroaryl, and C₆₋₁₂ cycloalkenyl; each moiety being optionally substituted with one or more substituents each independently selected from OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, and haloC₁₋₆ alkyl, or 2 substituents together with the atom to which they are attached may form a C₃₋₁₂ cycloalkyl, C₅₋₁₂ cycloalkenyl, heterocycloalkyl, or C₆₋₁₂ aryl; each of said formed C₃₋₁₂ cycloalkyl, C₅₋₁₂ cycloalkenyl, heterocycloalkyl or C₆₋₁₂ aryl may be optionally substituted by one or more C₁₋₆ alkyl;
Y² is —C(R⁷)R⁸— or —C(═O)—;
R⁷ is selected from the group comprising hydrogen, OH, SH, C₁₋₆ alkyl, C₃₋₁₂ cycloalkyl, C₁₋₆ alkoxy, amino, and halo;
R⁸ is hydrogen or C₁₋₆ alkyl;
or R⁷ and at least one substituent of R⁶ together with the carbon atom to which they are attached form a C₃₋₁₂ cycloalkyl, wherein the C₃₋₁₂ cycloalkyl can be optionally substituted with one or more substituents independently selected from the group comprising C₁₋₆ alkyl, OH, halo, C₃₋₁₂ cycloalkyl, C₂₋₆ alkenyl, C₁₋₆ alkoxy, oxo, ═CH₂, amino, mono- or di-C₁₋₆ alkylamino, amino C₁₋₆ alkyl, and haloC₁₋₆ alkyl, or two substituents together with the atom to which they are attached may form a C₃₋₁₂ cycloalkyl, a C₅₋₁₂ cycloalkenyl, a heterocycloalkyl or an C₆₋₁₂ aryl; each of the formed C₃₋₁₂ cycloalkyl, C₅₋₁₂ cycloalkenyl, heterocycloalkyl or C₆₋₁₂ aryl may be optionally substituted by one or more C₁₋₆ alkyl.

7. The G protein peptidomimetic or salt thereof of claim 1, wherein X₁₈ is a moiety of formula (Ic): 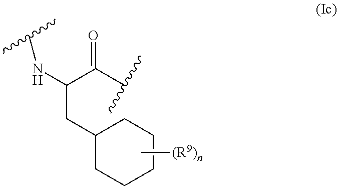
wherein
n is an integer selected from 0, 1, 2, 3, 4, or 5;
R⁹ is selected from the group comprising OH, halo, C₁₋₆ alkyl, C₃₋₁₂ cycloalkyl, C₂₋₆ alkenyl, C₁₋₆ alkoxy, oxo, ═CH₂, amino, mono- or di-C₁₋₆ alkylamino, and haloC₁-6 alkyl, or two R⁹ together with the atom to which they are attached may form a C₃₋₁₂ cycloalkyl, C₅₋₁₂ cycloalkenyl, heterocycloalkyl, or C₆₋₁₂ aryl; each of the formed C₃₋₁₂ cycloalkyl, C₅₋₁₂ cycloalkenyl, heterocycloalkyl or C₆₋₁₂ aryl may be optionally substituted by one or more C₁₋₆ alkyl.

8. The G protein peptidomimetic or salt thereof of claim 1, wherein X₁₈ is cyclohexylalanine (Cha).

9. The G protein peptidomimetic or salt thereof of claim 1, further comprising at least one basic amino acid at its N-terminus.

10. The G protein peptidomimetic or salt thereof of claim 9, wherein the at least one basic amino acid is selected from the group consisting of: lysine (K), histidine (H), arginine (R), and D-arginine.

11. The G protein peptidomimetic or salt thereof of claim 1, wherein the peptidomimetic comprises a triple lysine (K) at its N-terminus.

12. The G protein peptidomimetic or salt thereof of claim 1, further comprising a cell-penetrating peptide (CPP) at its N-terminus.

13. The G protein peptidomimetic or salt thereof of claim 1, wherein the peptidomimetic comprises an N-terminal modification.

14. The G protein peptidomimetic or salt thereof of claim 1, wherein the G protein peptidomimetic stabilizes a G protein-coupled receptor (GPCR) in an active conformational state.

15. The G protein peptidomimetic or salt thereof of claim 14, wherein the GPCR is a G_{q/11} protein coupled receptor.

16. The G protein peptidomimetic or salt thereof of claim 1, which is compound SBL-GQ-05 as defined by SEQ ID NO:5, compound SBL-GQ-06 as defined by SEQ ID NO:6, compound SBL-GQ-11 as defined by SEQ ID NO:11, compound SBL-GQ-12 as defined by SEQ ID NO:12, compound SBL-GQ-13 as defined by SEQ ID NO:41, compound SBL-GQ-14 as defined by SEQ ID NO:42, compound SBL-GQ-15 as defined by SEQ ID NO:43, or compound SBL-GQ-25 as defined by SEQ ID NO:51.

17. The G protein peptidomimetic or salt thereof of claim 1, wherein the G protein peptidomimetic or salt thereof is fused to a GPCR.

18. The G protein peptidomimetic or salt thereof of claim 1, wherein the G protein peptidomimetic or salt thereof is in a complex with a GPCR.

19. The G protein peptidomimetic or salt thereof of claim 18, wherein the complex further comprises a receptor ligand.

20. (canceled)

21. (canceled)

22. A method of capturing a GPCR in an active conformation, the method comprising:
contacting the G protein peptidomimetic or salt thereof of claim 1 with a GPCR, and
allowing the G protein peptidomimetic or salt thereof to bind to the GPCR, whereby the GPCR is captured in an active conformation.

23. (canceled)

24. A method of crystallizing a complex of a G protein peptidomimetic or salt thereof of claim 1 and a GPCR and optionally a ligand of the GPCR, the method comprising:
crystallizing a complex of the G protein peptidomimetic or salt thereof of claim 1 and the GPCR and optionally the ligand of the GPCR to form a crystal.

25. The method according to claim 24, further comprising obtaining the atomic coordinates of the crystal.

26. (canceled)

27. A screening method for identifying compounds capable of interacting with a GPCR the method comprising:
contacting the GPCR with a test compound and a G protein peptidomimetic or salt thereof of claim 1; and
evaluating binding of the test compound to the GPCR.

28. The G protein peptidomimetic or salt thereof of claim 1, wherein the G protein peptidomimetic allosterically modulates a GPCR.

29. The G protein peptidomimetic or salt thereof of claim 1, wherein the G protein peptidomimetic or salt thereof is comprised in a biosensor to detect conformational change of a GPCR, a biosensor to assess the localization and/or trafficking of a GPCR, and/or a biosensor to investigate a GPCR signaling pathway.


SEQUENCE LISTING (51 sequences)

SEQ ID NO: 1 - protein, 19 aa, synthetic construct
  MOD_RES 1: note = ACETYLATION
  source 1..19: note = SBL-GQ-01
FAAVKDTILQLNLKEYNLV

SEQ ID NO: 2 - protein, 22 aa, synthetic construct
  MOD_RES 1: note = ACETYLATION
  source 1..22: note = SBL-GQ-02
KKKFAAVKDTILQLNLKEYNLV

SEQ ID NO: 3 - protein, 19 aa, synthetic construct
  MOD_RES 1: note = ACETYLATION
  SITE 18: note = MISC_FEAT - wherein Xaa is cyclohexylalanine (Cha)
  source 1..19: note = SBL-GQ-03
FAAVKDTILQLNLKEYNXV

SEQ ID NO: 4 - protein, 22 aa, synthetic construct
  MOD_RES 1: note = ACETYLATION
  SITE 21: note = MISC_FEAT - wherein Xaa is cyclohexylalanine (Cha)
  source 1..22: note = SBL-GQ-04
KKKFAAVKDTILQLNLKEYNXV

SEQ ID NO: 5 - protein, 22 aa, synthetic construct
  MOD_RES 1: note = ACETYLATION
  SITE 6: note = MOD_RES - modified propargylglycine Pra which is linked to modified azidolysine Azk at position (10) via the reaction of the alkynyl side chain of a Pra with the azidated side-chain of an Azk
  SITE 10: note = MOD_RES - modified azidolysine Azk which is linked to modified propargylglycine Pra at position (6) via the reaction of the azidated side-chain of an Azk with the alkynyl side-chain of a Pra
  source 1..22: note = SBL-GQ-05
KKKFAXVKDXILQLNLKEYNLV

SEQ ID NO: 6 - protein, 22 aa, synthetic construct
  MOD_RES 1: note = ACETYLATION
  SITE 6: note = MOD_RES - modified propargylglycine Pra which is linked to modified azidolysine Azk at position (10) via the reaction of the alkynyl side chain of a Pra with the azidated side-chain of an Azk
  SITE 10: note = MOD_RES - modified azidolysine Azk which is linked to modified propargylglycine Pra at position (6) via the reaction of the azidated side-chain of an Azk with the alkynyl side-chain of a Pra
  SITE 21: note = MISC_FEAT - wherein Xaa is cyclohexylalanine (Cha)
  source 1..22: note = SBL-GQ-06
KKKFAXVKDXILQLNLKEYNXV

SEQ ID NO: 7 - protein, 19 aa, synthetic construct
  MOD_RES 1: note = ACETYLATION
  source 1..19: note = SBL-GQ-07
FNDCKDIILQMNLREYNLV

SEQ ID NO: 8 - protein, 22 aa, synthetic construct
  MOD_RES 1: note = ACETYLATION
  source 1..22: note = SBL-GQ-08
KKKFNDCKDIILQMNLREYNLV

SEQ ID NO: 9 - protein, 19 aa, synthetic construct
  MOD_RES 1: note = ACETYLATION
  SITE 18: note = MISC_FEAT - wherein Xaa is cyclohexylalanine (Cha)
  source 1..19: note = SBL-GQ-09
FNDCKDIILQMNLREYNXV

SEQ ID NO: 10 - protein, 22 aa, synthetic construct
  MOD_RES 1: note = ACETYLATION
  SITE 21: note = MISC_FEAT - wherein Xaa is cyclohexylalanine (Cha)
  source 1..22: note = SBL-GQ-10
KKKFNDCKDIILQMNLREYNXV

SEQ ID NO: 11 - protein, 22 aa, synthetic construct
  MOD_RES 1: note = ACETYLATION
  SITE 6: note = MOD_RES - modified propargylglycine Pra which is linked to modified azidolysine Azk at position (10) via the reaction of the alkynyl side chain of a Pra with the azidated side-chain of an Azk
  SITE 10: note = MOD_RES - modified azidolysine Azk which is linked to modified propargylglycine Pra at position (6) via the reaction of the azidated side-chain of an Azk with the alkynyl side-chain of a Pra
  source 1..22: note = SBL-GQ-11
KKKFNXCKDXILQMNLREYNLV

SEQ ID NO: 12 - protein, 22 aa, synthetic construct
  MOD_RES 1: note = ACETYLATION
  SITE 6: note = MOD_RES - modified propargylglycine Pra which is linked to modified azidolysine Azk at position (10) via the reaction of the alkynyl side chain of a Pra with the azidated side-chain of an Azk
  SITE 10: note = MOD_RES - modified azidolysine Azk which is linked to modified propargylglycine Pra at position (6) via the reaction of the azidated side-chain of an Azk with the alkynyl side-chain of a Pra
  SITE 21: note = MISC_FEAT - wherein Xaa is cyclohexylalanine (Cha)
  source 1..22: note = SBL-GQ-12
KKKFNXCKDXILQMNLREYNXV

SEQ ID NO: 13 - protein, 19 aa, synthetic construct
  source 1..19: note = alpha5 helix of Gq/11
FAAVKDTILQLNLKEYNLV

SEQ ID NO: 14 - protein, 19 aa, synthetic construct
  source 1..19: note = alpha5 helix of mini-Gq protein
FNDCKDIILQMNLREYNLV

SEQ ID NO: 15 - protein, 460 aa, Homo sapiens
MNTSAPPAVSPNITVLAPGKGPWQVAFIGITTGLLSLATVTGNLLVLISFKVNTELKTVNNYFLLSLACADLIIGTFSMNLYTTYLLMGHWALGTLACDLWLALDYVASNASVMNLLLISFDRYFSVTRPLSYRAKRTPRRAALMIGLAWLVSFVLWAPAILFWQYLVGERTVLAGQCYIQFLSQPIITFGTAMAAFYLPVTVMCTLYWRIYRETENRARELAALQGSETPGKGGGSSSSSERSQPGAEGSPETPPGRCCRCCRAPRLLQAYSWKEEEEEDEGSMESLTSSEGEEPGSEVVIKMPMVDPEAQAPTKQPPRSSPNTVKRPTKKGRDRAGKGQKPRGKEQLAKRKTFSLVKEKKAARTLSAILLAFILTWTPYNIMVLVSTFCKDCVPETLWELGYWLCYVNSTINPMCYALCNKAFRDTFRLLLLCRWDKRRWRKIPKRPGSVHRTPSRQC

SEQ ID NO: 16 - protein, 487 aa, Homo sapiens
MSLPNSSCLLEDKMCEGNKTTMASPQLMPLVVVLSTICLVTVGLNLLVLYAVRSERKLHTVGNLYIVSLSVADLIVGAVVMPMNILYLLMSKWSLGRPLCLFWLSMDYVASTASIFSVFILCIDRYRSVQQPLRYLKYRTKTRASATILGAWFLSFLWVIPILGWNHFMQQTSVRREDKCETDFYDVTWFKVMTAIINFYLPTLLMLWFYAKIYKAVRQHCQHRELINRSLPSFSEIKLRPENPKGDAKKPGKESPWEVLKRKPKDAGGGSVLKSPSQTPKEMKSPVVFSQEDDREVDKLYCFPLDIVHMQAAAEGSSRDYVAVNRSHGQLKTDEQGLNTHGASEISEDQMLGDSQSFSRTDSDTTTETAPGKGKLRSGSNTGLDYIKFTWKRLRSHSRQYVSGLHMNRERKAAKQLGFIMAAFILCWIPYFIFFMVIAFCKNCCNEHLHMFTIWLGYINSTLNPLIYPLCNENFKKTFKRILHIRS

SEQ ID NO: 17 - protein, 471 aa, Homo sapiens
MDILCEENTSLSSTTNSLMQLNDDTRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPLTIMVITYFLTIKSLQKEATLCVSDLGTRAKLASFSFLPQSSLSSEKLFQRSIHREPGSYTGRRTMQSISNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 18 - protein, 19 aa, synthetic construct
  SITE 2: note = MISC_FEAT - Xaa can be asparagine (N) or alanine (A)
  SITE 3: note = MISC_FEAT - Xaa can be moiety propargylglycine (Pra), moiety azidolysine (Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 4: note = MISC_FEAT - Xaa can be cysteine (C) or valine (V)
  SITE 7: note = MISC_FEAT - Xaa can be moiety propargylglycine (Pra), moiety azidolysine (Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 11: note = MISC_FEAT - Xaa can be methionine (M) or leucine (L)
  SITE 14: note = MISC_FEAT - Xaa can be arginine (R) or lysine (K)
  SITE 18: note = MISC_FEAT - Xaa can be leucine (L), moiety of formula (Ic), phenylalanine (F), tyrosine (Y), or tryptophan (W)
FXXXKDXILQXNLXEYNXV

SEQ ID NO: 19 - protein, 19 aa, synthetic construct
  SITE 3: note = MISC_FEAT - Xaa can be moiety propargylglycine (Pra), moiety azidolysine (Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 7: note = MISC_FEAT - Xaa can be moiety propargylglycine (Pra), moiety azidolysine (Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 18: note = MISC_FEAT - Xaa can be leucine (L), moiety of formula (Ic), phenylalanine (F), tyrosine (Y), or tryptophan (W)
FNXCKDXILQMNLREYNXV

SEQ ID NO: 20 - protein, 19 aa, synthetic construct
  SITE 3: note = MISC_FEAT - Xaa can be moiety propargylglycine (Pra), moiety azidolysine (Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 7: note = MISC_FEAT - Xaa can be moiety propargylglycine (Pra), moiety azidolysine (Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 18: note = MISC_FEAT - Xaa can be leucine (L), moiety of formula (Ic), phenylalanine (F), tyrosine (Y), or tryptophan (W)
FAXVKDXILQLNLKEYNXV

SEQ ID NO: 21 - protein, 19 aa, synthetic construct
  SITE 2: note = MISC_FEAT - Xaa can be asparagine (N) or alanine (A)
  SITE 3: note = MISC_FEAT - Xaa can be aspartic acid (D), alanine (A), propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 4: note = MISC_FEAT - Xaa can be cysteine (C) or valine (V)
  SITE 7: note = MISC_FEAT - Xaa can be isoleucine (I), threonine (T), propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 11: note = MISC_FEAT - Xaa can be methionine (M), leucine (L), propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 14: note = MISC_FEAT - Xaa can be arginine (R), lysine (K), propargylglycine (moiety Pra), azidolysine (moiety Azk),glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FXXXKDXILQXNLXXXXXX

SEQ ID NO: 22 - protein, 19 aa, synthetic construct
  SITE 3: note = MISC_FEAT - Xaa can be aspartic acid (D), propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 4: note = MISC_FEAT - Xaa can be cysteine (C) or valine (V)
  SITE 7: note = MISC_FEAT - Xaa can be isoleucine (I), propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 11: note = MISC_FEAT - Xaa can be methionine (M), propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 14: note = Xaa can be arginine (R), propargylglycine (moiety Pra), azidolysine (moiety Azk),glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FNXXKDXILQXNLXXXXXX

SEQ ID NO: 23 - protein, 19 aa, synthetic construct
  SITE 3: note = MISC_FEAT - Xaa can be alanine (A), propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 7: note = MISC_FEAT - Xaa can be threonine (T), propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 11: note = MISC_FEAT - Xaa can be leucine (L), propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 14: note = MISC_FEAT - Xaa can be lysine (K), propargylglycine (moiety Pra), azidolysine (moiety Azk),glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FAXVKDXILQXNLXXXXXX

SEQ ID NO: 24 - protein, 19 aa, synthetic construct
  SITE 3: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 4: note = MISC_FEAT - Xaa can be cysteine (C) or valine (V)
  SITE 7: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FNXXKDXILQMNLRXXXXX

SEQ ID NO: 25 - protein, 19 aa, synthetic construct
  SITE 3: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 7: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FAXVKDXILQLNLKXXXXX

SEQ ID NO: 26 - protein, 19 aa, synthetic construct
  SITE 4: note = MISC_FEAT - Xaa can be cysteine (C) or valine (V)
  SITE 7: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 11: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FNDXKDXILQXNLRXXXXX

SEQ ID NO: 27 - protein, 19 aa, synthetic construct
  SITE 7: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 11: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FAAVKDXILQXNLKXXXXX

SEQ ID NO: 28 - protein, 19 aa, synthetic construct
  SITE 4: note = MISC_FEAT - Xaa can be cysteine (C) or valine (V)
  SITE 11: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 14: note = MISC_FEAT - Xaa can be propargylglycine (moiety Pra), azidolysine (moiety Azk),glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FNDXKDIILQXNLXXXXXX

SEQ ID NO: 29 - protein, 19 aa, synthetic construct
  SITE 11: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 14: note = MISC_FEAT - Xaa can be propargylglycine (moiety Pra), azidolysine (moiety Azk),glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FAAVKDTILQXNLXXXXXX

SEQ ID NO: 30 - protein, 19 aa, synthetic construct
  SITE 4: note = MISC_FEAT - Xaa can be cysteine (C) or valine (V)
  SITE 7: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 14: note = MISC_FEAT - Xaa can be propargylglycine (moiety Pra), azidolysine (moiety Azk),glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FNDXKDXILQMNLXXXXXX

SEQ ID NO: 31 - protein, 19 aa, synthetic construct
  SITE 7: note = MISC_FEAT - Xaa can be propargylglycine (moiety Pra), azidolysine (moiety Azk), glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 14: note = MISC_FEAT - Xaa can be propargylglycine (moiety Pra), azidolysine (moiety Azk),glutamic acid (E), lysine (K), cysteine (C), (R)-2-(4-pentenyl)alanine (moiety R5), S-2-(4-pentenyl) alanine (moiety S5), or R-2-(7-octenyl) alanine (moiety R8)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FAAVKDXILQLNLXXXXXX

SEQ ID NO: 32 - protein, 19 aa, synthetic construct
  SITE 4: note = MISC_FEAT - Xaa can be cysteine (C) or valine (V)
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FNDXKDIILQMNLRXXXXX

SEQ ID NO: 33 - protein, 19 aa, synthetic construct
  SITE 15: note = MISC_FEAT - Xaa can be glutamic acid (E), homoglutamic acid (hGlu) or aspartic acid (D)
  SITE 16: note = MISC_FEAT - Xaa can be tyrosine (Y), 4-guanidinophenylalanine, tryptophan (W), phenylalanine (F), naphthylalanine, 1- naphthylalanine (1-Nal), or 2-naphthylalanine (2-Nal)
  SITE 17: note = MISC_FEAT - Xaa can be asparagine (N) or cysteine (C)
  SITE 18: note = MISC_FEAT - Xaa can be leucine (L), glutamic acid (E), moiety of formula (Ic), phenylalanine (F), tyrosine (Y), or tryptophan (W)
  SITE 19: note = MISC_FEAT - Xaa can be valine (V), aspartic acid (D), glutamic acid (E), D-aspartic acid or D-glutamic acid
FAAVKDTILQLNLKXXXXX

SEQ ID NO: 34 - skipped

SEQ ID NO: 35 - protein, 5 aa, synthetic construct
EYNLV

SEQ ID NO: 36 - protein, 8 aa, synthetic construct
  source 1..8: note = Arg8
RRRRRRRR

SEQ ID NO: 37 - protein, 4 aa, synthetic construct
  source 1..4: note = Arg4
RRRR

SEQ ID NO: 38 - protein, 13 aa, synthetic construct
  source 1..13: note = TAT peptide
GRKKRRQRRRPPQ

SEQ ID NO: 39 - protein, 9 aa, synthetic construct
  source 1..9: note = RW9
RRWWRRWRR

SEQ ID NO: 40 - protein, 6 aa, synthetic construct
  source 1..6: note = hydrophobic CPP
PFVYLI

SEQ ID NO: 41 - protein, 22 aa, synthetic construct
  source 1..22: note = SBL-GQ-13, SBL-GQ-16
  SITE 6: note = modified propargylglycine Pra which is linked to modified azidolysine Azk at position (10) via the reaction of the alkynyl side chain of Pra with the azidated side-chain of Azk
  SITE 10: note = modified azidolysine Azk which is linked to modified propargylglycine Pra at position (6) via the reaction of the azidated side-chain of an Azk with the alkynyl side-chain of a Pra
KKKFAXVKDXILQLNLKEYNLV

SEQ ID NO: 42 - protein, 23 aa, synthetic construct
  source 1..23: note = SBL-GQ-14
  SITE 7: note = modified propargylglycine Pra which is linked to modified azidolysine Azk at position (11) via the reaction of the alkynyl side chain of Pra with the azidated side-chain of Azk
  SITE 11: note = modified azidolysine Azk which is linked to modified propargylglycine Pra at position (7) via the reaction of the azidated side-chain of an Azk with the alkynyl side-chain of a Pra
RRRRFAXVKDXILQLNLKEYNLV

SEQ ID NO: 43 - protein, 27 aa, synthetic construct
  source 1..27: note = SBL-GQ-15
  SITE 11: note = modified propargylglycine Pra which is linked to modified azidolysine Azk at position (15) via the reaction of the alkynyl side chain of Pra with the azidated side-chain of Azk
  SITE 15: note = modified azidolysine Azk which is linked to modified propargylglycine Pra at position (11) via the reaction of the azidated side-chain of an Azk with the alkynyl side-chain of a Pra
RRRRRRRRFAXVKDXILQLNLKEYNLV

SEQ ID NO: 44 - protein, 23 aa, synthetic construct
  source 1..23: note = SBL-GQ-17, SBL-GQ-18
  SITE 1: note = aminohexanoic acid
  SITE 7: note = modified propargylglycine Pra which is linked to modified azidolysine Azk at position (11) via the reaction of the alkynyl side chain of Pra with the azidated side-chain of Azk
  SITE 11: note = modified azidolysine Azk which is linked to modified propargylglycine Pra at position (7) via the reaction of the azidated side-chain of an Azk with the alkynyl side-chain of a Pra
XKKKFAXVKDXILQLNLKEYNLV

SEQ ID NO: 45 - protein, 24 aa, synthetic construct
  source 1..24: note = SBL-GQ-19
  SITE 1: note = aminohexanoic acid
  SITE 8: note = modified propargylglycine Pra which is linked to modified azidolysine Azk at position (12) via the reaction of the alkynyl side chain of Pra with the azidated side-chain of Azk
  SITE 12: note = modified azidolysine Azk which is linked to modified propargylglycine Pra at position (8) via the reaction of the azidated side-chain of an Azk with the alkynyl side-chain of a Pra
XRRRRFAXVKDXILQLNLKEYNLV

SEQ ID NO: 46 - protein, 28 aa, synthetic construct
  source 1..28: note = SBL-GQ-20
  SITE 1: note = aminohexanoic acid
  SITE 12: note = modified propargylglycine Pra which is linked to modified azidolysine Azk at position (16) via the reaction of the alkynyl side chain of Pra with the azidated side-chain of Azk
  SITE 16: note = modified azidolysine Azk which is linked to modified propargylglycine Pra at position (12) via the reaction of the azidated side-chain of an Azk with the alkynyl side-chain of a Pra
XRRRRRRRRFAXVKDXILQLNLKEYNLV

SEQ ID NO: 47 - protein, 24 aa, synthetic construct
  source 1..24: note = SBL-GQ-21
  SITE 2: note = aminohexanoic acid
  SITE 8: note = modified propargylglycine Pra which is linked to modified azidolysine Azk at position (12) via the reaction of the alkynyl side chain of Pra with the azidated side-chain of Azk
  SITE 12: note = modified azidolysine Azk which is linked to modified propargylglycine Pra at position (8) via the reaction of the azidated side-chain of an Azk with the alkynyl side-chain of a Pra
  MOD_RES 1: note = cysteine side chain is linked to a fluorochrome
CXKKKFAXVKDXILQLNLKEYNLV

SEQ ID NO: 48 - protein, 25 aa, synthetic construct
  source 1..25: note = SBL-GQ-22
  SITE 2: note = aminohexanoic acid
  SITE 9: note = modified propargylglycine Pra which is linked to modified azidolysine Azk at position (13) via the reaction of the alkynyl side chain of Pra with the azidated side-chain of Azk
  SITE 13: note = modified azidolysine Azk which is linked to modified propargylglycine Pra at position (9) via the reaction of the azidated side-chain of an Azk with the alkynyl side-chain of a Pra
  MOD_RES 1: note = cysteine side chain is linked to a fluorochrome
CXRRRRFAXVKDXILQLNLKEYNLV

SEQ ID NO: 49 - protein, 29 aa, synthetic construct
  source 1..29: note = SBL-GQ-23
  MOD_RES 1: note = cysteine side chain is linked to a fluorochrome
  SITE 2: note = aminohexanoic acid
  SITE 13: note = modified propargylglycine Pra which is linked to modified azidolysine Azk at position (17) via the reaction of the alkynyl side chain of Pra with the azidated side-chain of Azk
  SITE 17: note = modified azidolysine Azk which is linked to modified propargylglycine Pra at position (13) via the reaction of the azidated side-chain of an Azk with the alkynyl side-chain of a Pra
CXRRRRRRRRFAXVKDXILQLNLKEYNLV

SEQ ID NO: 50 - protein, 30 aa, synthetic construct
  source 1..30: note = SBL-GQ-24
  MOD_RES 1: note = cysteine side chain is linked to a fluorochrome
  SITE 2: note = aminohexanoic acid
  SITE 14: note = modified propargylglycine Pra which is linked to modified azidolysine Azk at position (18) via the reaction of the alkynyl side chain of Pra with the azidated side-chain of Azk
  SITE 18: note = modified azidolysine Azk which is linked to modified propargylglycine Pra at position (14) via the reaction of the azidated side-chain of an Azk with the alkynyl side-chain of a Pra
CXRRWWRRWRRFAXVKDXILQLNLKEYNLV

SEQ ID NO: 51 - protein, 28 aa, synthetic construct
  source 1..28: note = SBL-GQ-25
  SITE 12: note = modified propargylglycine Pra which is linked to modified azidolysine Azk at position (16) via the reaction of the alkynyl side chain of Pra with the azidated side-chain of Azk
  SITE 16: note = modified azidolysine Azk which is linked to modified propargylglycine Pra at position (12) via the reaction of the azidated side-chain of an Azk with the alkynyl side-chain of a Pra
RRWWRRWRRFAXVKDXILQLNLKEYNLV